(19) United States
(12) Patent Application Publication
(10) Pub. No.: US 2020/0039930 A1
(43) Pub. Date: Feb. 6, 2020

(54) PROLINE AMIDE COMPOUNDS AND THEIR AZETIDINE ANALOGUES CARRYING A SPECIFICALLY SUBSTITUTED BENZYL RADICAL

(71) Applicants: AbbVie Deutschland GmbH & Co. KG, Wiesbaden (DE); AbbVie Inc., North Chicago, IL (US)

(72) Inventors: Gisela Backfisch, Ludwigshafen (DE); Margaretha Henrica Maria Bakker, Ludwigshafen (DE); Lawrence Black, North Chicago (IL); Wilfried Braje, Ludwigshafen (DE); Karla Drescher, Ludwigshafen (DE); Thomas Erhard, Ludwigshafen (DE); Andreas Haupt, Ludwigshafen (DE); Carolin Hoft, Ludwigshafen (DE); Andreas Kling, Ludwigshafen (DE); Viktor Lakics, Ludwigshafen (DE); Helmut Mack, Ludwigshafen (DE); Frank Oellien, Ludwigshafen (DE); Ana Lucia Relo, Ludwigshafen (DE)

(21) Appl. No.: 16/496,256

(22) PCT Filed: Mar. 20, 2018
(86) PCT No.: PCT/US2018/023376

§ 371 Date: Sep. 20, 2019

Related U.S. Application Data
(60) Provisional application No. 62/474,407, filed on Mar. 21, 2017.

(51) Int. Cl. C07D 207/16

(52) U.S. Cl. CPC C07D 207/16

(57) ABSTRACT

The present invention relates to proline amide compounds and their azetidine derivatives of formula I wherein the variables are as defined in the claims and the description. The invention further relates to a pharmaceutical composition containing such compounds, to their use as modulators, especially agonists or partial agonists, of the 5-HT_{2C} receptor, their use for preparing a medicament for the prevention or treatment of conditions and disorders which respond to the modulation of 5-HT_{2C} receptor, to a method for preventing or treating conditions and disorders which respond to the modulation of the 5-HT_{2C} receptor, and processes for preparing such compounds and compositions.

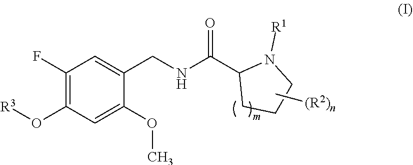

### FIELD OF THE INVENTION

The present invention relates to proline amide compounds and their azetidine derivatives carrying on the amide nitrogen atom a benzyl radical the phenyl ring of which carries a fluorine atom, a methoxy radical and an O-bound radical containing fluoro substiries xto a pharmaceutical composition containing such compounds, to their use as modulators, especially agonists or partial agonists, of the 5-HT_{2C} receptor, their use for preparing a medicament for the prevention or treatment of conditions and disorders which respond to the modulation of 5-HT_{2C} receptor, to a method for preventing or treating conditions and disorders which respond to the modulation of the 5-HT_{2C} receptor, and processes for preparing such compounds and compositions.

### BACKGROUND OF THE INVENTION

Diseases, disorders and conditions where 5-HT_{2C} modulation is desired are for example depression, anxiety, schizophrenia, bipolar disorder, obsessive compulsive disorder, migraine, pain, epilepsy, substance abuse, eating disorders, obesity, diabetes, erectile dysfunction and others.

Serotonin (5-hydroxytryptamine, 5-HT), a monoamine neurotransmitter and local hormone, is formed by the hydroxylation and decarboxylation of tryptophan. The greatest concentration is found in the enterochromaffin cells of the gastrointestinal tract, the remainder being predominantly present in platelets and in the Central Nervous System (CNS). 5-HT is implicated in a vast array of physiological and pathophysiological pathways. In the periphery, it contracts a number of smooth muscles and induces endothelium-dependent vasodilation. In the CNS, it is believed to be involved in a wide range of functions, including the control of appetite, mood, anxiety, hallucinations, sleep, vomiting and pain perception.

Neurons that secrete 5-HT are termed serotonergic. The function of 5-HT is exerted upon its interaction with specific (serotonergic) neurons. Seven types of 5-HT receptors have been identified: 5-HT₁ (with subtypes 5-HT_{1A}, 5-HT_{1B}, 5-HT_{1D}, 5-HT_{1E} and 5-HT_{1F}), 5-HT₂ (with subtypes 5-HT_{2A}, 5-HT_{2B} and 5-HT_{2C}), 5-HT₃, 5-HT₄, 5-HT₅ (with subtypes 5-HT_{5A} and 5-HT_{5B}), 5-HT₆ and 5-HT₇. Most of these receptors are coupled to G-proteins that affect the activities of adenylate cyclase or phospholipase Cγ.

Alterations in the activity of multiple neurotransmitter receptor systems (dopamine, serotonin, glutamate, GABA, acetylcholine) have been implicated in the manifestation of the symptoms of schizophrenia. The most widely accepted “Dopamine Hypothesis of Schizophrenia” in its simplest form states that the positive symptoms of this pathology relate to a functional hyperactivity of the mesolimbic dopaminergic system, while the negative and cognitive aspects can be traced to a functional hypoactivity of the mesocortical dopaminergic projections. Atypical antipsychotics block the mesolimbic dopaminergic neurotransmission, thereby controlling positive symptoms, with little or no effect on the nigrostriatal system, leading to less induction of extrapyramidal side effects (EPS).

Primary negative and cognitive symptoms of schizophrenia reflect a dysfunction of the frontal cortex (“hypofrontality”), which is thought to be induced by a decreased tone in the mesocortical dopaminergic projection field [Davis K L, Kahn R S, Ko G and Davidson M (1991). Dopamine in schizophrenia: a review and re-conceptualization. Am J Psychiatry 148: 1474-86. Weinberger D R and Berman K F (1996). Prefrontal function in schizophrenia: confounds and controversies. Philos Trans R Soc Lond B Biol Sci 351: 1495-503]. Agents that selectively enhance dopamine levels in the cortex have the potential to address the negative symptoms of this disorder. Atypical antipsychotics lack robust efficacy against negative and cognitive components of the schizophrenic syndrome.

The schizophrenic symptomatology is further complicated by the occurrence of drug-induced so-called secondary negative symptoms and cognitive impairment, which are difficult to distinguish from primary negative and cognitive symptoms [Remington G and Kapur S (2000). Atypical antipsychotics: are some more atypical than others?Psychopharmacol 148: 3-15]. The occurrence of secondary negative symptoms not only limits therapeutic efficacy but also, together with these side effects, negatively afphrenic xpatient compliance.

It may thus be hypothesized that a novel mechanistic approach that blocks dopaminergic neurotransmission in the limbic system but does not affect the striatal and pituitary projection fields, and stimulates frontocortical projection fields, would provide an efficacious treatment for all parts of the schizophrenic pathology, including its positive, negative and cognitive symptoms. Moreover, a selective compound that is substantially free of the ancillary pharmacology that characterizes current agents would be expected to avoid a variety of off-target side effects that plague current treatments such as extrapyramidal side effects (EPS) and weight gain.

The 5-HT_{2C} receptor, previously named 5-HT1C, is a G-protein-coupled receptor, which couples to multiple cellular effector systems including the phospholipase C, A and D pathways. It is found primarily in the brain and its distribution is particularly high in the plexus choroideus, where it is assumed to control cerebrospinal fluid production [Kaufman M J, Hirata F (1996) Cyclic GMP inhibits phosphoinositide turnover in choroid plexus: evidence for interactions between second messengers concurrently triggered by 5-HT_{2C} receptors. Neurosci Lett 206:153-156]. Very high levels were also found in the retrosplenial, piriform and entorhinal cortex, anterior olfactory nucleus, lateral septal nucleus, subthalamic nucleus, amygdala, subiculum and ventral part of CA3, lateral habenula, substantia nigra pars compacta, several brainstem nuclei and the whole grey matter of the spinal cord [Pompeiano M, Palacios J M, Mengod G (1994). Distribution of the serotonin 5-HT2 receptor family mRNAs: comparison between 5-HT_{2A} and 5-HT_{2C} receptors. Brain Res Mol Brain Res 23:163-178]. A comparison of the distribution of 5-HT_{2C} mRNA with that of 5-HT_{2C} protein in monkey and human brains has revealed both pre- and postsynaptic localization [Lopez-Gimenez J F, Mengod G, Palacios J M, Vilaro M T (2001) Regional distribution and cellular localization of 5-HT_{2C} receptor mRNA in monkey brain: comparison with [³H]mesulergine binding sites and choline acetyltransferase mRNA. Synapse 42:12-26].

It is anticipated that modulation of the 5-HT_{2C} receptor will improve disorders such as depression, anxiety, schizophrenia, cognitive deficits of schizophrenia, obsessive compulsive disorder, bipolar disorder, neuropsychiatric symptoms in Parkinson' disease, in Alzheimer's disease or Lewy Body dementia, migraine, epilepsy, substance abuse, eating disorders, obesity, diabetes, sexual dysfunction/erectile dysfunction, sleep disorders, psoriasis, Parkinson's disease, pain conditions and disorders, and spinal cord injury, smoking cessation, ocular hypertension and Alzheimer's disease. Modulators of the 5-HT_{2C} receptor are also shown to be useful in the modulation of bladder function, including the prevention or treatment of urinary incontinence.

Compounds with a structure similar to the compounds of the present invention have been described in WO 2012/053186 and WO 2006/055184.

K. K.-C. Liu et al. describe in Bioorganic & Medicinal Chemistry Letters 2010, 20, 2365-2369 substituted N-benzyl proline amides to be highly selective 5-HT_{2c} agonists and useful for the treatment of obesity. However, it has been found that the metabolic stability of these N-benzyl proline amides is not satisfactory.

It was thus an object of the present invention to provide compounds with a comparable activity on the 5-HT_{2c} receptor, but with a better metabolic stability than the compounds described by K. K.-C. Liu et al.

It is further desirable that the compounds have low affinity to adrenergic receptors, such as the α₁-adrenergic receptor, histamine receptors, such as the H₁-receptor, and dopaminergic receptors, such as the D₂-receptor, in order to avoid or reduce side effects associated with modulation of these receptors, such as postural hypotension, reflex tachycardia, potentiation of the antihypertensive effect of prazosin, terazosin, doxazosin and labetalol or dizziness associated with the blockade of the α₁-adrenergic receptor, weight gain, sedation, drowsiness or potentiation of central depressant drugs associated with the blockade of the H₁-receptor, or extrapyramidal movement disorder, such as dystonia, parkinsonism, akathisia, tardive dyskinesia or rabbit syndrome, or endocrine effects, such as prolactin elevation (galactorrhea, gynecomastia, mentstrual changes, sexual dysfunction in males), associated with the blockade of the D₂-receptor, and even more important no induction of weight gain in combination with severe metabolic dysfunction found for marketed antipsychotic drugs.

It is moreover desirable that the compounds have low affinity or alternatively an antagonistic effect to/on other serotonergic receptors, especially the 5-HT_{2A} and/or 5-HT_{2B} receptors, in order to avoid or reduce side effects associated with modulation of these receptors, such as changes (thickening) of the heart tissue associated with agonism at the 5-HT_{2B} receptor, and psychotomimetic effect induced by agonism at the 5-HT_{2A} receptor. Ideally they should show an agonistic action on the 5-HT_{2C} receptor, an antagonistic action on the 5-HT_{2A} receptor or alternatively no affinity to the 5-HT_{2A} receptor and no affinity to the 5-HT_{2B} receptor or alternatively an antagonistic action on the 5-HT_{2B} receptor. Even more ideally the compounds should display an agonistic action on the 5-HT_{2C} receptor in combination with an antagonistic action on the 5-HT_{2A} receptor and no affinity to the 5-HT_{2B} receptor.

Besides the affinity and selectivity for the 5-HT_{2C} receptor and a sufficiently high metabolic stability (for example determined from the half-lives, measured in vitro, in liver microsomes from various species such as rat or human), further properties may be advantageous for the treatment and/or prophylaxis of 5-HT_{2C}-related disorders, such as, for example:

1.) no or only low inhibition of cytochrome P450 (CYP) enzymes: cytochrome P450 (CYP) is the name for a superfamily of heme proteins having enzymatic activity (oxidase). They are also particularly important for the degradation (metabolism) of foreign substances such as drugs or xenobiotics in mammalian organisms. The principal representatives of the types and subtypes of CYP in the human body are: CYP 1A2, CYP 2C9, CYP 2D6 and CYP 3A4. If CYP 3A4 inhibitors (e.g. grapefruit juice, cimetidine, erythromycin) are used at the same time as medicinal substances which are degraded by this enzyme system and thus compete for the same binding site on the enzyme, the degradation thereof may be slowed down and thus effects and side effects of the administered medicinal substance may be undesirably enhanced;

2.) a suitable solubility in water (in mg/mL);

3.) suitable pharmacokinetics (time course of the concentration of the compound of the invention in plasma or in tissue, for example brain). The pharmacokinetics can be described by the following parameters: half-life (in h), volume of distribution (in l·kg−1), plasma clearance (in l·h−1·kg−1), AUC (area under the curve, area under the concentration-time curve, in ng·h·l−1), oral bioavailability (the dose-normalized ratio of AUC after oral administration and AUC after intravenous administration), the so-called brain-plasma ratio (the ratio of AUC in brain tissue and AUC in plasma);

4.) no or only low blockade of the hERG channel: compounds which block the hERG channel may cause a prolongation of the QT interval and thus lead to serious disturbances of cardiac rhythm (for example so-called “torsade de pointes”). The potential of compounds to block the hERG channel can be determined by means of the displacement assay with radiolabelled dofetilide which is described in the literature (G. J. Diaz et al., Journal of Pharmacological and Toxicological Methods, 50 (2004), 187 199). A smaller IC50 in this dofetilide assay means a greater probability of potent hERG blockade. In addition, the blockade of the hERG channel can be measured by electrophysiological experiments on cells which have been transfected with the hERG channel, by so-called whole-cell patch clamping (G. J. Diaz et al., Journal of Pharmacological and Toxicological Methods, 50 (2004), 187-199).

It was an object of the present invention to provide compounds for the treatment or prophylaxis of various 5-HT_{2C}-related diseases. The compounds were intended to have a high affinity to the 5-HT_{2C} receptor and be potent and efficacious 5-HT_{2C} agonists. In addition, the compounds of the invention were intended to have sufficiently high metabolic stability. Further they should show low affinity on other serotonergic receptors, and especially the lack of potent agonistic effect (antagonism preferred) on the 5-HT_{2A} and/or 5-HT_{2B} receptors. Additionally they should have one or more of those advantages mentioned under 1.) to 4.), and especially under 3.) (oral bioavailability in vivo).

The present invention provides compounds which have an affinity for the 5-HT₂c receptor, thus allowing the treatment of disorders related to or affected by the 5-HT₂c receptor.

### SUMMARY OF THE INVENTION

The present invention relates to proline amide compounds and their azetidine derivatives carrying on the amide nitrogen atom a benzyl radical the phenyl ring of which carries a fluorine atom, a methoxy radical and an O-bound radical containing fluoro substitution, to a pharmaceutical composition containing such compounds, to their use as modulators, especially agonists or partial agonists, of the 5-HT_{2C} receptor, their use for preparing a medicament for the prevention or treatment of conditions and disorders which respond to the modulation of 5-HT_{2C} receptor, to a method for preventing or treating conditions and disorders which respond to the modulation of 5-HT_{2C} receptor, and processes for preparing such compounds and compositions.

In one aspect, the present invention relates to compounds of the formula (I):

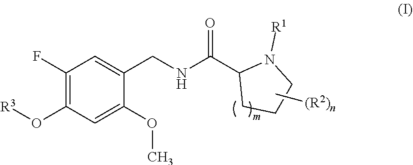

wherein
R¹ is hydrogen or methyl;
R² is fluoro or methyl;
R³ is selected from the group consisting of C₃-C₇-cycloalkyl which carries 1, 2, 3, 4, 5 or 6 substituents selected from the group consisting of fluoro and fluorinated C₁-C₄-alkyl; fluorinated C₁-C₈-alkyl; C₃-C₇-cycloalkyl-C₁-C₄-alkyl, where the cycloalkyl moiety carries 1, 2, 3, 4, 5 or 6 substituents selected from the group consisting of fluoro and fluorinated C₁-C₄-alkyl; phenyl-C₁-C₄-alkyl, where the phenyl ring carries 1, 2, 3, 4 or 5 substituents selected from the group consisting of fluoro and fluorinated C₁-C₄-alkyl, and may additionally carry one or more substituents selected from the group consisting of Cl, methyl and methoxy; and hetaryl-C₁-C₄-alkyl, where hetaryl is a 5- or 6-membered monocyclic heteroaromatic ring containing 1, 2 or 3 heteroatoms selected from the group consisting of N, O and S as ring members, where the heteroaryl ring carries 1, 2, 3, 4 or 5 substituents selected from the group consisting of fluoro and fluorinated C₁-C₄-alkyl, and may additionally carry one or more substituents selected from the group consisting of Cl, methyl and methoxy;
m is 0 or 1; and
n is 0 or 1;
   or a stereoisomer or a pharmaceutically acceptable salt thereof; or the compound of the general formula I, wherein at least one of the hydrogen atoms has been replaced by deuterium.

In another aspect, the invention relates to a pharmaceutical composition comprising a therapeutically effective amount of at least one compound of formula I or a stereoisomer or a pharmaceutically acceptable salt thereof or of at least one compound of the general formula I, wherein at least one of the hydrogen atoms has been replaced by deuterium, in combination with at least one pharmaceutically acceptable carrier and/or auxiliary substance.

In yet another aspect, the invention relates to a compound of formula I or a stereoisomer or a pharmaceutically acceptable salt thereof or to a compound of the general formula I, wherein at least one of the hydrogen atoms has been replaced by deuterium, for use as a medicament.

In yet another aspect, the invention relates to a compound of formula I or a stereoisomer or a pharmaceutically acceptable salt thereof or to a compound of the general formula I, wherein at least one of the hydrogen atoms has been replaced by deuterium, for use in the treatment of disorders which responds to the modulation of the 5-HT₂c receptor.

In yet another aspect, the invention relates to a compound of formula I or a stereoisomer or a pharmaceutically acceptable salt thereof or to a compound of the general formula I, wherein at least one of the hydrogen atoms has been replaced by deuterium, for use in the treatment of disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

In yet another aspect, the invention relates to the use of a compound of formula I or of a stereoisomer or a pharmaceutically acceptable salt thereof or of a compound of the general formula I, wherein at least one of the hydrogen atoms has been replaced by deuterium, for the manufacture of a medicament for the treatment of disorders which respond to the modulation of the 5-HT_{2C} receptor.

In yet another aspect, the invention relates to the use of a compound of formula I or of a stereoisomer or a pharmaceutically acceptable salt thereof or of a compound of the general formula I, wherein at least one of the hydrogen atoms has been replaced by deuterium for the manufacture of a medicament for the treatment of disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

In yet another aspect, the invention relates to a method for treating disorders which respond to the modulation of the 5-HT_{2C} receptor, which method comprises administering to a subject in need thereof at least one compound of formula I or a stereoisomer or a pharmaceutically acceptable salt thereof or at least one compound of the general formula I, wherein at least one of the hydrogen atoms has been replaced by deuterium.

In yet another aspect, the invention relates to a method for treating disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disor-alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance, which method comprises administering to a subject in need thereof at least one compound of formula I or a stereoisomer or a pharmaceutically acceptable salt thereof or at least one compound of the general formula I, wherein at least one of the hydrogen atoms has been replaced by deuterium.

In yet another aspect, the invention relates to a method for modulating 5HT₂c receptor activity in a subject, in particular in a subject suffering of one of the above-listed disorders.

### DETAILED DESCRIPTION

The compounds of the formula I may exist in different spatial arrangements. For example, if the compounds possess one or more centers of asymmetry or polysubstituted rings, or may exist as different tautomers, the present invention contemplates the possible use of enantiomeric mixtures, in particular racemates, diastereomeric mixtures and tautomeric mixtures, as well as the respective essentially pure enantiomers, diastereomers and/or tautomers of the compounds of formula I and/or their salts.

One center of chirality is for example the carbon atom via which the pyrrolidine or azetidine ring is bound to C(O). Other centers of chirality are for example asymmetry centers in the radical R³. Moreover, if R² is present (n=0), the carbon atom of the pyrrolidine or azetidine ring carrying the substituent R² is a center of chirality.

It is likewise possible to use physiologically tolerated salts of the compounds of the formula I, especially acid addition salts with physiologically tolerated acids. Examples of suitable physiologically tolerated organic and inorganic acids are hydrochloric acid, hydrobromic acid, phosphoric acid, sulfuric acid, acetic acid, trifluoroacetic acid, C₁-C₄-alkylsulfonic acids, such as methanesulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Other utilizable acids are described in Fortschritte der Arzneimittelforschung [Advances in drug research], Volume 10, pages 224 et seq., Birkhauser Verlag, Basel and Stuttgart, 1966.

Amide/imidic acid tautomerism in the C(O)—NH group may be present.

Compounds wherein m is 1 are pyrrolidine rings:

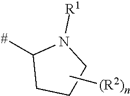

Compounds wherein m is 0 are azetidine rings:

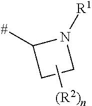

The organic moieties mentioned in the above definitions of the variables are, like the term halogen, collective terms for individual listings of the individual group members. The prefix Cₙ-Cₘ indicates in each case the possible number of carbon atoms in the group.

The term “alkyl” as used herein and in the alkyl moieties of alkoxy and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”) or 1 to 8 (“C₁-C₈-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl and the position isomers thereof.

The term “fluorinated alkyl” as used herein refers to straight-chain or branched alkyl groups having 1 or 2 (“fluorinated C₁-C₂-alkyl”), 1 to 3 (“fluorinated C₁-C₃-alkyl”), 1 to 4 (“fluorinated C₁-C₄-alkyl”), 1 to 6 (“fluorinated C₁-C₆-alkyl”) or 1 to 8 (“fluorinated C₁-C₈-alkyl”) carbon atoms (as mentioned above), where some or all of the hydrogen atoms in these groups are replaced by fluorine atoms. Fluorinated methyl is fluoromethyl, difluoromethyl or trifluoromethyl. Fluorinated C₁-C₂-alkyl is an alkyl group having 1 or 2 carbon atoms (as mentioned above), where at least one of the hydrogen atoms, e.g. 1, 2, 3, 4 or 5 hydrogen atoms in these groups are replaced by fluorine atoms, such as fluoromethyl, difluoromethyl, trifluoromethyl, 1-fluoroethyl, (R)-1-fluoroethyl, (S)-1-fluoroethyl, 2-fluoroethyl, 2,2-difluoroethyl, 2,2,2-trifluoroethyl, or pentafluoroethyl. Fluorinated C₁-C₄-alkyl is a straight-chain or branched alkyl group having 1 to 4 carbon atoms (as mentioned above), where at least one of the hydrogen atoms, e.g. 1, 2, 3, 4 or 5 hydrogen atoms in these groups are replaced by fluorine atoms. Examples are, apart those listed above for fluorinated C₁-C₂-alkyl, 1-fluoropropyl, (R)-1-fluoropropyl, (S)-1-fluoropropyl, 2-fluoropropyl, (R)-2-fluoropropyl, (S)-2-fluoropropyl, 3-fluoropropyl, 1,1-difluoropropyl, 2,2-difluoropropyl, 1,2-difluoropropyl, 2,3-difluoropropyl, 1,3-difluoropropyl, 3,3-difluoropropyl, 1,1,2-trifluoropropyl, 1,2,2-trifluoropropyl, 1,2,3-trifluoropropyl, 2,2,3-trifluoropropyl, 3,3,3-trifluoropropyl, 2,2,3,3-tetrafluoropropyl, 2,2,3,3,3-pentafluoropropyl, 1,1,1-trifluoroprop-2-yl, 2-fluoro-1-methylethyl, (R)-2-fluoro-1-methylethyl, (S)-2-fluoro-1-methylethyl, 2,2-difluoro-1-methylethyl, (R)-2,2-difluoro-1-methylethyl, (S)-2,2-difluoro-1-methylethyl, 1,2-difluoro-1-methylethyl, (R)-1,2-difluoro-1-methylethyl, (S)-1,2-difluoro-1-methylethyl, 2,2,2-trifluoro-1-methylethyl, (R)-2,2,2-trifluoro-1-methylethyl, (S)-2,2,2-trifluoro-1-methylethyl, 2-fluoro-1-(fluoromethyl)ethyl, 1-(difluoromethyl)-2,2-difluoroethyl, 1-(trifluoromethyl)-2,2,2-trifluoroethyl, 1-(trifluoromethyl)-1,2,2,2-tetrafluoroethyl, 1-fluorobutyl, (R)-1-fluorobutyl, (S)-1-fluorobutyl, 2-fluorobutyl, (R)-2-fluorobutyl, (S)-2-fluorobutyl, 3-fluorobutyl, (R)-3-fluorobutyl, (S)-3-fluorobutyl, 4-fluorobutyl, 1,1-difluorobutyl, 2,2-difluorobutyl, 3,3-difluorobutyl, 4,4-difluorobutyl, 4,4,4-trifluorobutyl, 3,3,4,4-tetrafluorobutyl, 3,4,4,4-tetrafluorobutyl, 2,2,4,4,4-pentafluorobutyl, 3,3,4,4,4-pentafluorobutyl, 2,2,3,4,4,4-hexafluorobutyl, 1-methyl-2,2-3,3-tetrafluoropropyl and the like. Fluorinated C₁-C₆-alkyl is a straight-chain or branched alkyl group having 1 to 6 carbon atoms (as mentioned above), where at least one of the hydrogen atoms, e.g. 1, 2, 3, 4 or 5 hydrogen atoms in these groups are replaced by fluorine atoms. Examples are, apart those listed above for fluorinated C₁-C₄-alkyl, 1-fluoropentyl, (R)-1-fluoropentyl, (S)-1-fluoropentyl, 2-fluoropentyl, (R)-2-fluoropentyl, (S)-2-fluoropentyl, 3-fluoropentyl, (R)-3-fluoropentyl, (S)-3-fluoropentyl, 4-fluoropentyl, (R)-4-fluoropentyl, (S)-4-fluoropentyl, 5-fluoropentyl, (R)-5-fluoropentyl, (S)-5-fluoropentyl, 2-methyl-4,4,4-trifluorobutyl, 1-fluorohexyl, (R)-1-fluorohexyl, (S)-1-fluorohexyl, 2-fluorohexyl, (R)-2-fluorohexyl, (S)-2-fluorohexyl, 3-fluorohexyl, (R)-3-fluorohexyl, (S)-3-fluorohexyl, 4-fluorohexyl, (R)-4-fluorohexyl, (S)-4-fluorohexyl, 5-fluorohexyl, (R)-5-fluorohexyl, (S)-5-fluorohexyl, 6-fluorohexyl, (R)-6-fluorohexyl, (S)-6-fluorohexyl, and the like. Fluorinated C₁-C₈-alkyl is a straight-chain or branched alkyl group having 1 to 8 carbon atoms (as mentioned above), where at least one of the hydrogen atoms, e.g. 1, 2, 3, 4 or 5 hydrogen atoms in these groups are replaced by fluorine atoms. “Fluorinated C₂-C₆-alkyl, where the carbon atom of the alkyl group which bound to O does not carry any fluorine atom” is for example 2-fluoroethyl, 2,2-difluoroethyl, 2,2,2-trifluoroethyl, 2-fluoropropyl, (R)-2-fluoropropyl, (S)-2-fluoropropyl, 3-fluoropropyl, 2,2-difluoropropyl, 2,3-difluoropropyl, 3,3-difluoropropyl, 2,2,3-trifluoropropyl, 3,3,3-trifluoropropyl, 2,2,3,3-tetrafluoropropyl, 2,2,3,3,3-pentafluoropropyl, 2-fluoro-1-methylethyl, (R)-2-fluoro-1-methylethyl, (S)-2-fluoro-1-methylethyl, 2,2-difluoro-1-methylethyl, (R)-2,2-difluoro-1-methylethyl, (S)-2,2-difluoro-1-methylethyl, 2,2,2-trifluoro-1-methylethyl, (R)-2,2,2-trifluoro-1-methylethyl, (S)-2,2,2-trifluoro-1-methylethyl, 2-fluoro-1-(fluoromethyl)ethyl, 1-(difluoromethyl)-2,2-difluoroethyl, 1-(trifluoromethyl)-2,2,2-trifluoroethyl, 1-(trifluoromethyl)-1,2,2,2-tetrafluoroethyl, 2-fluorobutyl, (R)-2-fluorobutyl, (S)-2-fluorobutyl, 3-fluorobutyl, (R)-3-fluorobutyl, (S)-3-fluorobutyl, 4-fluorobutyl, 2,2-difluorobutyl, 3,3-difluorobutyl, 4,4-difluorobutyl, 4,4,4-trifluorobutyl, 3,3,4,4-tetrafluorobutyl, 3,4,4,4-tetrafluorobutyl, 2,2,4,4,4-pentafluorobutyl, 3,3,4,4,4-pentafluorobutyl, 2,2,3,4,4,4-hexafluorobutyl, 1-methyl-2,2-3,3-tetrafluoropropyl, 2-fluoropentyl, (R)-2-fluoropentyl, (S)-2-fluoropentyl, 3-fluoropentyl, (R)-3-fluoropentyl, (S)-3-fluoropentyl, 4-fluoropentyl, (R)-4-fluoropentyl, (S)-4-fluoropentyl, 5-fluoropentyl, (R)-5-fluoropentyl, (S)-5-fluoropentyl, 2-methyl-4,4,4-trifluorobutyl, 2-fluorohexyl, (R)-2-fluorohexyl, (S)-2-fluorohexyl, 3-fluorohexyl, (R)-3-fluorohexyl, (S)-3-fluorohexyl, 4-fluorohexyl, (R)-4-fluorohexyl, (S)-4-fluorohexyl, 5-fluorohexyl, (R)-5-fluorohexyl, (S)-5-fluorohexyl, 6-fluorohexyl, (R)-6-fluorohexyl, (S)-6-fluorohexyl, and the like.

“C₄-C₆-Cycloalkyl” refers to monocyclic saturated hydrocarbon radicals having 4 to 6 carbon atoms. Examples are cyclobutyl, cyclopentyl and cyclohexyl. “C₃-C₇-Cycloalkyl” refers to monocyclic saturated hydrocarbon radicals having 3 to 7 carbon atoms. Examples are cyclopropyl, cyclobutyl, cyclopentyl, cyclohexyl and cycloheptyl.

“C₃-C₇-Cycloalkyl-methyl” refers to monocyclic saturated hydrocarbon radicals having 3 to 7 carbon atoms as defined above which are bound to the remainder of the molecule via a methyl group. Examples are cyclopropylmethyl, cyclobutylmethyl, cyclopentylmethyl, cyclohexylmethyl and cycloheptylmethyl.

“C₃-C₇-Cycloalkyl-C₁-C₄-alkyl” refers to monocyclic saturated hydrocarbon radicals having 3 to 7 carbon atoms as defined above which are bound to the remainder of the molecule via a C₁-C₄-alkyl group. Examples are cyclopropylmethyl, cyclobutylmethyl, cyclopentylmethyl, cyclohexylmethyl, cycloheptylmethyl, 1-cyclopropylethyl, 1-cyclobutylethyl, 1-cyclopentylethyl, 1-cyclohexylethyl, 1-cycloheptylethyl, 2-cyclopropylethyl, 2-cyclobutylethyl, 2-cyclopentylethyl, 2-cyclohexylethyl, 2-cycloheptylethyl, 1-cyclopropylpropyl, 1-cyclobutylpropyl, 1-cyclopentylpropyl, 1-cyclohexylpropyl, 1-cycloheptylpropyl, 2-cyclopropylpropyl, 2-cyclobutylpropyl, 2-cyclopentylpropyl, 2-cyclohexylpropyl, 2-cycloheptylpropyl, 3-cyclopropylpropyl, 3-cyclobutylpropyl, 3-cyclopentylpropyl, 3-cyclohexylpropyl, 3-cycloheptylpropyl, 2-cyclopropyl-1-methylethyl, 2-cyclobutyl-1-methylethyl, 2-cyclopentyl-1-methylethyl, 2-cyclohexyl-1-methylethyl, 2-cycloheptyl-1-methylethyl, 1-cyclopropylbutyl, 1-cyclobutylbutyl, 1-cyclopentylbutyl, 1-cyclohexylbutyl, 1-cycloheptylbutyl, 2-cyclopropylbutyl, 2-cyclobutylbutyl, 2-cyclopentylbutyl, 2-cyclohexylbutyl, 2-cycloheptylbutyl, 3-cyclopropylbutyl, 3-cyclobutylbutyl, 3-cyclopentylbutyl, 3-cyclohexylbutyl, 3-cycloheptylbutyl, 4-cyclopropylbutyl, 4-cyclobutylbutyl, 4-cyclopentylbutyl, 4-cyclohexylbutyl, 4-cycloheptylbutyl and the like.

Phenyl-C₁-C₄-alkyl” refers to phenyl bound to the remainder of the molecule via a C₁-C₄-alkyl group. Examples are benzyl, 1-phenylethyl, 2-phenylethyl (phenethyl), 1-phenylpropyl, 2-phenylpropyl, 3-phenylpropyl,

Examples for hetaryl (or heteroaryl) being a 5- or 6-membered monocyclic heteroaromatic ring containing 1 heteroatom selected from the group consisting of N and O as ring member are 2-furyl, 3-furyl, 2-thienyl, 3-thienyl, 1-pyrrolyl, 2-pyrrolyl, 3-pyrrolyl, 2-pyridinyl, 3-pyridinyl and 4-pyridinyl.

Examples for hetaryl (or heteroaryl) being a 5- or 6-membered monocyclic heteroaromatic ring containing 1, 2 or 3 heteroatoms selected from the group consisting of N, O and S as ring member are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,2,3-triazol-1-yl, 1,2,3-triazol-2-yl, 1,2,3-triazol-4-yl, 1,3,4-triazol-1-yl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl and 1,3,5-triazin-2-yl.

Hetaryl-C₁-C₄-alkyl is a 5- or 6-membered monocyclic heteroaromatic ring containing 1, 2 or 3 heteroatoms selected from the group consisting of N, O and S as ring members (examples therefor see above) which is bound via a C₁-C₄-alkyl group to the remainder of the molecule.

The remarks made above and in the following with respect to preferred aspects of the invention, e.g. to preferred meanings of the variables R¹, R², R³, m and n of compounds I, to preferred compounds I and to preferred embodiments of the method or the use according to the invention, apply in each case on their own or in particular to combinations thereof.

In one embodiment, R¹ is hydrogen. In another embodiment, R¹ is methyl. Preferably, however, R¹ is hydrogen.

In one preferred embodiment, R³ is C₄-C₆-cycloalkyl which carries 1, 2, 3 or 4 substituents selected from the group consisting of fluoro and fluorinated methyl. In particular, R³ is C₄-C₆-cycloalkyl which carries 1 or 2 substituents selected from the group consisting of fluoro and fluorinated methyl. Specifically, R³ is C₄-C₆-cycloalkyl which carries 1 or 2 fluoro substituents or one substituent which is selected from fluorinated methyl (i.e. from CH₂F, CHF₂ or CF₃).

In another preferred embodiment, R³ is fluorinated C₂-C₆-alkyl, where the carbon atom of the alkyl group which is bound to O does not carry any fluorine atom. In particular, R³ is fluorinated C₃-C₅-alkyl, where the carbon atom of the alkyl group which is bound to O does not carry any fluorine atom. Specifically, the fluorinated alkyl group contains 3 to 6 fluorine atoms.

In another preferred embodiment, R³ is C₃-C₇-cycloalkyl-methyl, where the cycloalkyl moiety carries 1, 2, 3, 4, 5 or 6 substituents selected from the group consisting of fluoro and fluorinated methyl. In particular, R³ is C₃-C₆-cycloalkyl-methyl, where the cycloalkyl moiety carries 1, 2, 3, 4, 5 or 6 substituents selected from the group consisting of fluoro and fluorinated methyl. More particularly, R³ is C₃-C₆-cycloalkyl-methyl, where the cycloalkyl moiety carries 1, 2, 3, 4, 5 or 6 fluoro substituents or carries one substituent which is selected from fluorinated methyl (i.e. from CH₂F, CHF₂ or CF₃).

In another preferred embodiment, R³ is phenyl-C₁-C₂-alkyl, where the phenyl ring carries 1, 2, 3 or 4 substituents selected from the group consisting of fluoro and fluorinated methyl, and may additionally carry one Cl substituent. In particular, R³ is benzyl or phenethyl, where the phenyl ring in the two last-mentioned radicals carries 1, 2, 3 or fluorine atoms and optionally also a chlorine atom or carries one substituent which is selected from fluorinated methyl (i.e. from CH₂F, CHF₂ or CF₃).

In another preferred embodiment, R³ is hetaryl-C₁-C₂-alkyl, where hetaryl is a 5- or 6-membered monocyclic heteroaromatic ring containing 1 heteroatom selected from the group consisting of N and O as ring member, where the heteroaryl ring carries 1 or 2 substituents selected from the group consisting of fluoro and fluorinated methyl. In particular, R³ is hetaryl-methyl, where hetaryl is a 5- or 6-membered monocyclic heteroaromatic ring containing 1 heteroatom selected from the group consisting of N and O as ring member, where the heteroaryl ring carries 1 or 2 fluoro substituents or carries one substituent which is selected from fluorinated methyl (i.e. from CH₂F, CHF₂ or CF₃).

In one embodiment, m is 1. In another embodiment, m is 0. Preferably, however, m is 1.

In a preferred embodiment, n is 0.

In one embodiment, in compounds I at least one of the hydrogen atoms of the moiety

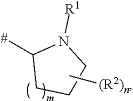

where # is the attachment point to C(O), has been replaced by a deuterium atom. In particular, at least one of the hydrogen atoms bound to a carbon ring atom of the pyrrolidine (m=1) or azetidine (m=0) ring of the above moiety has been replaced by a deuterium atom. Specifically, in the above moiety, all hydrogen atoms bound to carbon ring atoms have been replaced by deuterium atoms.

In a particular embodiment, the compounds of formula I are compounds of formula I.1

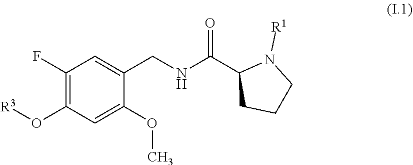

where R¹ and R³ have one of the above general or, in particular, one of the above preferred meanings.

Examples of preferred compounds are compounds of the following formulae Ia.1 to Ia.6 and the stereoisomers thereof and the pharmaceutically acceptable salts thereof, where R³ is as defined in table A. For an individual compound R³ corresponds in each case to one row of table A. Moreover, the meanings mentioned below for R³ are per se, independently of the combination in which they are mentioned, a particularly preferred embodiment of this substituent.

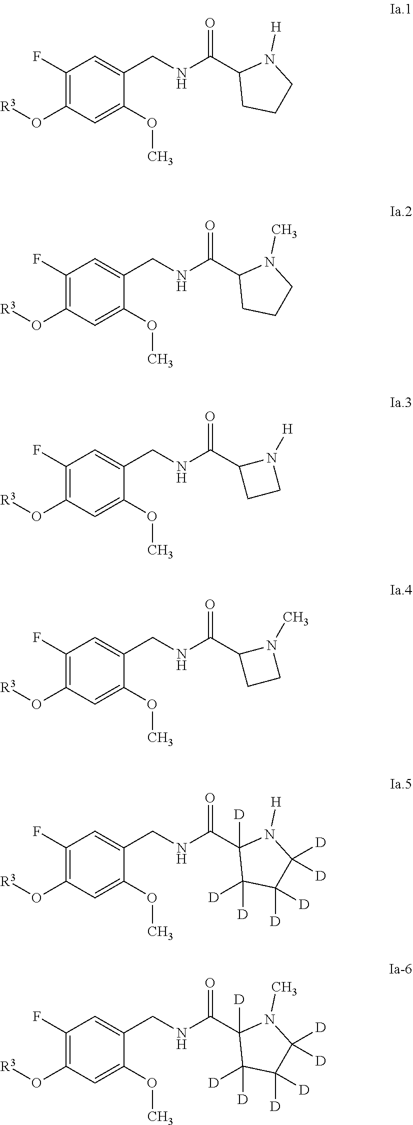

TABLE A

| No. | R³ |
|---|---|
| 1. | CH₂F |
| 2. | CHF₂ |
| 3. | CF₃ |
| 4. | CH₂CH₂F |
| 5. | CH₂CHF₂ |
| 6. | CH₂CF₃ |
| 7. | CHFCH₃ |
| 8. | CF₂CH₃ |
| 9. | CHFCH₂F |
| 10. | CHFCHF₂ |
| 11. | CHFCF₃ |
| 12. | CF₂CH₂F |
| 13. | CF₂CHF₂ |
| 14. | CF₂CF₃ |
| 15. | CH₂CH₂CH₂F |
| 16. | CH₂CH₂CHF₂ |
| 17. | CH₂CH₂CF₃ |
| 18. | CH₂CHFCH₃ |
| 19. | CH₂CF₂CH₃ |
| 20. | CH₂CHFCH₂F |
| 21. | CH₂CHFCHF₂ |
| 22. | CH₂CHFCF₃ |
| 23. | CH₂CF₂CH₂F |
| 24. | CH₂CF₂CHF₂ |
| 25. | CH₂CF₂CF₃ |
| 26. | CHFCH₂CH₃ |
| 27. | CHFCH₂CH₂F |
| 28. | CHFCH₂CHF₂ |
| 29. | CHFCH₂CF₃ |
| 30. | CHFCHFCH₃ |
| 31. | CHFCF₂CH₃ |
| 32. | CHFCHFCH₂F |
| 33. | CHFCHFCHF₂ |
| 34. | CHFCHFCF₃ |
| 35. | CHFCF₂CH₂F |
| 36. | CHFCF₂CHF₂ |
| 37. | CHFCF₂CF₃ |
| 38. | CF₂CH₂CH₃ |
| 39. | CF₂CH₂CH₂F |
| 40. | CF₂CH₂CHF₂ |
| 41. | CF₂CH₂CF₃ |
| 42. | CF₂CHFCH₃ |
| 43. | CF₂CF₂CH₃ |
| 44. | CF₂CHFCH₂F |
| 45. | CF₂CHFCHF₂ |
| 46. | CF₂CHFCF₃ |
| 47. | CF₂CF₂CH₂F |
| 48. | CF₂CF₂CHF₂ |
| 49. | CF₂CF₂CF₃ |
| 50. | CF(CH₃)₂ |
| 51. | CH(CH₃)CH₂F |
| 52. | CH(CH₃)CHF₂ |
| 53. | CH(CH₃)CF₃ |
| 54. | CF(CH₃)CH₂F |
| 55. | CF(CH₃)CHF₂ |
| 56. | CF(CH₃)CF₃ |
| 57. | CH(CF₃)₂ |
| 58. | CF(CF₃)₂ |
| 59. | CH₂CH₂CH₂CH₂F |
| 60. | CH₂CH₂CH₂CHF₂ |
| 61. | CH₂CH₂CH₂CF₃ |
| 62. | CH₂CH₂CHFCH₃ |
| 63. | CH₂CH₂CF₂CH₃ |
| 64. | CH₂CH₂CHFCH₂F |
| 65. | CH₂CH₂CHFCHF₂ |
| 66. | CH₂CH₂CHFCF₃ |
| 67. | CH₂CH₂CF₂CH₂F |
| 68. | CH₂CH₂CF₂CHF₂ |
| 69. | CH₂CH₂CF₂CF₃ |
| 70. | CH₂CHFCH₂CH₃ |
| 71. | CH₂CHFCH₂CH₂F |
| 72. | CH₂CHFCH₂CHF₂ |
| 73. | CH₂CHFCH₂CF₃ |
| 74. | CH₂CHFCHFCH₃ |
| 75. | CH₂CHFCF₂CH₃ |
| 76. | CH₂CHFCHFCH₂F |
| 77. | CH₂CHFCHFCHF₂ |
| 78. | CH₂CHFCHFCF₃ |
| 79. | CH₂CHFCF₂CH₂F |
| 80. | CH₂CHFCF₂CHF₂ |
| 81. | CH₂CHFCF₂CF₃ |
| 82. | CH₂CF₂CH₂CH₃ |
| 83. | CH₂CF₂CH₂CH₂F |
| 84. | CH₂CF₂CH₂CHF₂ |
| 85. | CH₂CF₂CH₂CF₃ |
| 86. | CH₂CF₂CHFCH₃ |
| 87. | CH₂CF₂CF₂CH₃ |
| 88. | CH₂CF₂CHFCH₂F |
| 89. | CH₂CF₂CHFCHF₂ |
| 90. | CH₂CF₂CHFCF₃ |
| 91. | CH₂CF₂CF₂CH₂F |
| 92. | CH₂CF₂CF₂CHF₂ |
| 93. | CH₂CF₂CF₂CF₃ |
| 94. | CHFCH₂CH₂CH₃ |
| 95. | CHFCH₂CH₂CH₂F |
| 96. | CHFCH₂CH₂CHF₂ |
| 97. | CHFCH₂CH₂CF₃ |
| 98. | CHFCH₂CHFCH₃ |
| 99. | CHFCH₂CF₂CH₃ |
| 100. | CHFCH₂CHFCH₂F |
| 101. | CHFCH₂CHFCHF₂ |
| 102. | CHFCH₂CHFCF₃ |
| 103. | CHFCH₂CF₂CH₂F |
| 104. | CHFCH₂CF₂CHF₂ |
| 105. | CHFCH₂CF₂CF₃ |
| 106. | CHFCHFCH₂CH₃ |
| 107. | CHFCHFCH₂CH₂F |
| 108. | CHFCHFCH₂CHF₂ |
| 109. | CHFCHFCH₂CF₃ |
| 110. | CHFCHFCHFCH₃ |
| 111. | CHFCHFCF₂CH₃ |
| 112. | CHFCHFCHFCH₂F |
| 113. | CHFCHFCHFCHF₂ |
| 114. | CHFCHFCHFCF₃ |
| 115. | CHFCHFCF₂CH₂F |
| 116. | CHFCHFCF₂CHF₂ |
| 117. | CHFCHFCF₂CF₃ |
| 118. | CHFCF₂CH₂CH₃ |
| 119. | CHFCF₂CH₂CH₂F |
| 120. | CHFCF₂CH₂CHF₂ |
| 121. | CHFCF₂CH₂CF₃ |
| 122. | CHFCF₂CHFCH₃ |
| 123. | CHFCF₂CF₂CH₃ |
| 124. | CHFCF₂CHFCH₂F |
| 125. | CHFCF₂CHFCHF₂ |
| 126. | CHFCF₂CHFCF₃ |
| 127. | CHFCF₂CF₂CH₂F |
| 128. | CHFCF₂CF₂CHF₂ |
| 129. | CHFCF₂CF₂CF₃ |
| 130. | CF₂CH₂CH₂CH₂F |
| 131. | CF₂CH₂CH₂CHF₂ |
| 132. | CF₂CH₂CH₂CF₃ |
| 133. | CF₂CH₂CHFCH₃ |
| 134. | CF₂CH₂CF₂CH₃ |
| 135. | CF₂CH₂CHFCH₂F |
| 136. | CF₂CH₂CHFCHF₂ |
| 137. | CF₂CH₂CHFCF₃ |
| 138. | CF₂CH₂CF₂CH₂F |
| 139. | CF₂CH₂CF₂CHF₂ |
| 140. | CF₂CH₂CF₂CF₃ |
| 141. | CF₂CHFCH₂CH₃ |
| 142. | CF₂CHFCH₂CH₂F |
| 143. | CF₂CHFCH₂CHF₂ |
| 144. | CF₂CHFCH₂CF₃ |
| 145. | CF₂CHFCHFCH₃ |
| 146. | CF₂CHFCF₂CH₃ |
| 147. | CF₂CHFCHFCH₂F |
| 148. | CF₂CHFCHFCHF₂ |
| 149. | CF₂CHFCHFCF₃ |
| 150. | CF₂CHFCF₂CH₂F |
| 151. | CF₂CHFCF₂CHF₂ |
| 152. | CF₂CHFCF₂CF₃ |
| 153. | CF₂CF₂CH₂CH₃ |
| 154. | CF₂CF₂CH₂CH₂F |
| 155. | CF₂CF₂CH₂CHF₂ |
| 156. | CF₂CF₂CH₂CF₃ |
| 157. | CF₂CF₂CHFCH₃ |
| 158. | CF₂CF₂CF₂CH₃ |
| 159. | CF₂CF₂CHFCH₂F |
| 160. | CF₂CF₂CHFCHF₂ |
| 161. | CF₂CF₂CHFCF₃ |
| 162. | CF₂CF₂CF₂CH₂F |
| 163. | CF₂CF₂CF₂CHF₂ |
| 164. | CF₂CF₂CF₂CF₃ |
| 165. | CH(CH₃)CH₂CH₂F |
| 166. | CH(CH₃)CH₂CHF₂ |
| 167. | CH(CH₃)CH₂CF₃ |
| 168. | CH(CH₃)CHFCH₃ |
| 169. | CH(CH₃)CHFCH₂F |
| 170. | CH(CH₃)CHFCHF₂ |
| 171. | CH(CH₃)CHFCF₃ |
| 172. | CH(CH₃)CF₂CH₂F |
| 173. | CH(CH₃)CF₂CHF₂ |
| 174. | CH(CH₃)CF₂CF₃ |
| 175. | CH₂CH(CH₃)CH₂F |
| 176. | CH₂CH(CH₃)CHF₂ |
| 177. | CH₂CH(CH₃)CF₃ |
| 178. | CH₂CH(CF₃)₂ |
| 179. | C(CH₃)₂(CF₃) |
| 180. | C(CF₃)₂(CH₃) |
| 181. | C(CF₃)₃ |
| 182. | CH₂CH₂CH₂CH₂F |
| 183. | CH₂CH₂CH₂CH₂CHF₂ |
| 184. | CH₂CH₂CH₂CH₂CF₃ |
| 185. | CH₂CH₂CH₂CHFCH₃ |
| 186. | CH₂CH₂CH₂CF₂CH₃ |
| 187. | CH₂CH₂CH₂CHFCH₂F |
| 188. | CH₂CH₂CH₂CHFCHF₂ |
| 189. | CH₂CH₂CH₂CHFCF₃ |
| 190. | CH₂CH₂CH₂CF₂CH₂F |
| 191. | CH₂CH₂CH₂CF₂CHF₂ |
| 192. | CH₂CH₂CH₂CF₂CF₃ |
| 193. | CH₂CH₂CHFCH₂CH₃ |
| 194. | CH₂CH₂CHFCH₂CH₂F |
| 195. | CH₂CH₂CHFCH₂CHF₂ |
| 196. | CH₂CH₂CHFCH₂CF₃ |
| 197. | CH₂CH₂CHFCHFCH₃ |
| 198. | CH₂CH₂CHFCF₂CH₃ |
| 199. | CH₂CH₂CHFCHFCH₂F |
| 200. | CH₂CH₂CHFCHFCHF₂ |
| 201. | CH₂CH₂CHFCHFCF₃ |
| 202. | CH₂CH₂CHFCF₂CH₂F |
| 203. | CH₂CH₂CHFCF₂CHF₂ |
| 204. | CH₂CH₂CHFCF₂CF₃ |
| 205. | CH₂CH₂CF₂CH₂CH₃ |
| 206. | CH₂CH₂CF₂CH₂CH₂F |
| 207. | CH₂CH₂CF₂CH₂CHF₂ |
| 208. | CH₂CH₂CF₂CH₂CF₃ |
| 209. | CH₂CH₂CF₂CHFCH₃ |
| 210. | CH₂CH₂CF₂CF₂CH₃ |
| 211. | CH₂CH₂CF₂CHFCH₂F |
| 212. | CH₂CH₂CF₂CHFCHF₂ |
| 213. | CH₂CH₂CF₂CHFCF₃ |
| 214. | CH₂CH₂CF₂CF₂CH₂F |
| 215. | CH₂CH₂CF₂CF₂CHF₂ |
| 216. | CH₂CH₂CF₂CF₂CF₃ |
| 217. | CH₂CHFCH₂CH₂CH₃ |
| 218. | CH₂CHFCH₂CH₂CH₂F |
| 219. | CH₂CHFCH₂CH₂CHF₂ |
| 220. | CH₂CHFCH₂CH₂CF₃ |
| 221. | CH₂CHFCH₂CHFCH₃ |
| 222. | CH₂CHFCH₂CF₂CH₃ |
| 223. | CH₂CHFCH₂CHFCH₂F |
| 224. | CH₂CHFCH₂CHFCHF₂ |
| 225. | CH₂CHFCH₂CHFCF₃ |
| 226. | CH₂CHFCH₂CF₂CH₂F |
| 227. | CH₂CHFCH₂CF₂CHF₂ |
| 228. | CH₂CHFCH₂CF₂CF₃ |
| 229. | CH₂CHFCHFCH₂CH₃ |
| 230. | CH₂CHFCHFCH₂CH₂F |
| 231. | CH₂CHFCHFCH₂CHF₂ |
| 232. | CH₂CHFCHFCH₂CF₃ |
| 233. | CH₂CHFCHFCHFCH₃ |
| 234. | CH₂CHFCHFCF₂CH₃ |
| 235. | CH₂CHFCHFCHFCH₂F |
| 236. | CH₂CHFCHFCHFCHF₂ |
| 237. | CH₂CHFCHFCHFCF₃ |
| 238. | CH₂CHFCHFCF₂CH₂F |
| 239. | CH₂CHFCHFCF₂CHF₂ |
| 240. | CH₂CHFCHFCF₂CF₃ |
| 241. | CH₂CHFCF₂CH₂CH₃ |
| 242. | CH₂CHFCF₂CH₂CH₂F |
| 243. | CH₂CHFCF₂CH₂CHF₂ |
| 244. | CH₂CHFCF₂CH₂CF₃ |
| 245. | CH₂CHFCF₂CHFCH₃ |
| 246. | CH₂CHFCF₂CF₂CH₃ |
| 247. | CH₂CHFCF₂CHFCH₂F |
| 248. | CH₂CHFCF₂CHFCHF₂ |
| 249. | CH₂CHFCF₂CHFCF₃ |
| 250. | CH₂CHFCF₂CF₂CH₂F |
| 251. | CH₂CHFCF₂CF₂CHF₂ |
| 252. | CH₂CHFCF₂CF₂CF₃ |
| 253. | CH₂CF₂CH₂CH₂CH₂F |
| 254. | CH₂CF₂CH₂CH₂CHF₂ |
| 255. | CH₂CF₂CH₂CH₂CF₃ |
| 256. | CH₂CF₂CH₂CHFCH₃ |
| 257. | CH₂CF₂CH₂CF₂CH₃ |
| 258. | CH₂CF₂CH₂CHFCH₂F |
| 259. | CH₂CF₂CH₂CHFCHF₂ |
| 260. | CH₂CF₂CH₂CHFCF₃ |
| 261. | CH₂CF₂CH₂CF₂CH₂F |
| 262. | CH₂CF₂CH₂CF₂CHF₂ |
| 263. | CH₂CF₂CH₂CF₂CF₃ |
| 264. | CH₂CF₂CHFCH₂CH₃ |
| 265. | CH₂CF₂CHFCH₂CH₂F |
| 266. | CH₂CF₂CHFCH₂CHF₂ |
| 267. | CH₂CF₂CHFCH₂CF₃ |
| 268. | CH₂CF₂CHFCHFCH₃ |
| 269. | CH₂CF₂CHFCF₂CH₃ |
| 270. | CH₂CF₂CHFCHFCH₂F |
| 271. | CH₂CF₂CHFCHFCHF₂ |
| 272. | CH₂CF₂CHFCHFCF₃ |
| 273. | CH₂CF₂CHFCF₂CH₂F |
| 274. | CH₂CF₂CHFCF₂CHF₂ |
| 275. | CH₂CF₂CHFCF₂CF₃ |
| 276. | CH₂CF₂CF₂CH₂CH₃ |
| 277. | CH₂CF₂CF₂CH₂CH₂F |
| 278. | CH₂CF₂CF₂CH₂CHF₂ |
| 279. | CH₂CF₂CF₂CH₂CF₃ |
| 280. | CH₂CF₂CF₂CHFCH₃ |
| 281. | CH₂CF₂CF₂CF₂CH₃ |
| 282. | CH₂CF₂CF₂CHFCH₂F |
| 283. | CH₂CF₂CF₂CHFCHF₂ |
| 284. | CH₂CF₂CF₂CHFCF₃ |
| 285. | CH₂CF₂CF₂CF₂CH₂F |
| 286. | CH₂CF₂CF₂CF₂CHF₂ |
| 287. | CH₂CF₂CF₂CF₂CF₃ |
| 288. | CH(CH₃)CH₂CH₂CH₂F |
| 289. | CH(CH₃)CH₂CH₂CHF₂ |
| 290. | CH(CH₃)CH₂CH₂CF₃ |
| 291. | CH(CH₃)CH₂CHFCH₃ |
| 292. | CH(CH₃)CH₂CF₂CH₃ |
| 293. | CH(CH₃)CH₂CHFCH₂F |
| 294. | CH(CH₃)CH₂CHFCHF₂ |
| 295. | CH(CH₃)CH₂CHFCF₃ |
| 296. | CH(CH₃)CH₂CF₂CH₂F |
| 297. | CH(CH₃)CH₂CF₂CHF₂ |
| 298. | CH(CH₃)CH₂CF₂CF₃ |
| 299. | CH₂CH(CH₃)CH₂CH₂F |
| 300. | CH₂CH(CH₃)CH₂CHF₂ |
| 301. | CH₂CH(CH₃)CH₂CF₃ |
| 302. | CH₂CH(CH₃)CHFCH₃ |
| 303. | CH₂CH(CH₃)CHFCHF₂ |
| 304. | CH₂CH(CH₃)CHFCF₃ |
| 305. | CH₂CH(CH₃)CF₂CH₂F |
| 306. | CH₂CH(CH₃)CF₂CHF₂ |
| 307. | CH₂CH(CH₃)CF₂CF₃ |
| 308. | CH₂CH(CF₃)CH₂CH₃ |
| 309. | CH₂CH₂CH(CH₃)CH₂F |
| 310. | CH₂CH₂CH(CH₃)CHF₂ |
| 311. | CH₂CH₂CH(CH₃)CF₃ |
| 312. | CH₂CH₂CH(CF₃)₂ |
| 313. | A.1 |
| 314. | A.2 |
| 315. | A.3 |
| 316. | A.4 |
| 317. | A.5 |
| 318. | A.6 |
| 319. | A.7 |
| 320. | A.8 |
| 321. | A.9 |
| 322. | A.10 |
| 323. | A.11 |
| 324. | A.12 |
| 325. | A.13 |
| 326. | A.14 |
| 327. | A.15 |
| 328. | A.16 |
| 329. | A.17 |
| 330. | A.18 |
| 331. | A.19 |
| 332. | A.20 |
| 333. | A.21 |
| 334. | A.22 |
| 335. | A.23 |
| 336. | A.24 |
| 337. | A.25 |
| 338. | A.26 |
| 339. | A.27 |
| 340. | A.28 |
| 341. | A.29 |
| 342. | A.30 |
| 343. | A.31 |
| 344. | A.32 |
| 345. | A.33 |
| 346. | A.34 |
| 347. | A.35 |
| 348. | A.36 |
| 349. | A.37 |
| 350. | A.38 |
| 351. | A.39 |
| 352. | A.40 |
| 353. | A.41 |
| 354. | A.42 |
| 355. | A.43 |
| 356. | A.44 |
| 357. | A.45 |
| 358. | A.46 |
| 359. | A.47 |
| 360. | A.48 |
| 361. | A.49 |
| 362. | A.50 |
| 363. | A.51 |
| 364. | A.52 |
| 365. | A.53 |
| 366. | A.54 |
| 367. | A.55 |
| 368. | A.56 |
| 369. | A.57 |
| 370. | A.58 |
| 371. | —CH₂-A.1 |
| 372. | —CH₂-A.2 |
| 373. | —CH₂-A.3 |
| 374. | —CH₂-A.4 |
| 375. | —CH₂-A.5 |
| 376. | —CH₂-A.6 |
| 377. | —CH₂-A.7 |
| 378.y | —CH₂-A.8 |
| 379. | —CH₂-A.9 |
| 380. | —CH₂-A.10 |
| 381. | —CH₂-A.11 |
| 382. | —CH₂-A.12 |
| 383. | —CH₂-A.13 |
| 384. | —CH₂-A.14 |
| 385. | —CH₂-A.15 |
| 386. | —CH₂-A.16 |
| 387. | —CH₂-A.17 |
| 388. | —CH₂-A.18 |
| 389. | —CH₂-A.19 |
| 390. | —CH₂-A.20 |
| 391. | —CH₂-A.21 |
| 392. | —CH₂-A.22 |
| 393. | —CH₂-A.23 |
| 394. | —CH₂-A.24 |
| 395. | —CH₂-A.25 |
| 396. | —CH₂-A.26 |
| 397. | —CH₂-A.27 |
| 398. | —CH₂-A.28 |
| 399. | —CH₂-A.29 |
| 400. | —CH₂-A.30 |
| 401. | —CH₂-A.31 |
| 402. | —CH₂-A.32 |
| 403. | —CH₂-A.33 |
| 404. | —CH₂-A.34 |
| 405. | —CH₂-A.35 |
| 406. | —CH₂-A.36 |
| 407. | —CH₂-A.37 |
| 408. | —CH₂-A.38 |
| 409. | —CH₂-A.39 |
| 410. | —CH₂-A.40 |
| 411. | —CH₂-A.41 |
| 412. | —CH₂-A.42 |
| 413. | —CH₂-A.43 |
| 414. | —CH₂-A.44 |
| 415. | —CH₂-A.45 |
| 416. | —CH₂-A.46 |
| 417. | —CH₂-A.47 |
| 418. | —CH₂-A.48 |
| 419. | —CH₂-A.49 |
| 420. | —CH₂-A.50 |
| 421. | —CH₂-A.51 |
| 422. | —CH₂-A.52 |
| 423. | —CH₂-A.53 |
| 424. | —CH₂-A.54 |
| 425. | —CH₂-A.55 |
| 426. | —CH₂-A.56 |
| 427. | —CH₂-A.57 |
| 428. | —CH₂-A.58 |
| 429. | —CH₂-A.59 |
| 430. | —CH₂-A.60 |
| 431. | —CH₂-A.61 |
| 432. | —CH₂-A.62 |
| 433. | —CH₂-A.63 |
| 434. | —CH₂-A.64 |
| 435. | —CH₂-A.65 |
| 436. | —CH₂-A.66 |
| 437. | —CH₂-A.67 |
| 438. | —CH₂-A.68 |
| 439. | —CH₂-A.69 |
| 440. | —CH₂-A.70 |
| 441. | —CH₂-A.71 |
| 442. | —CH₂-A.72 |
| 443. | —CH₂-A.73 |
| 444. | —CH₂-A.74 |
| 445. | —CH₂-A.75 |
| 446. | —CH₂-A.76 |
| 447. | —CH₂-A.77 |
| 448. | —CH₂-A.78 |
| 449. | —CH₂-A.79 |
| 450. | —CH₂-A.80 |
| 451. | —CH₂-A.81 |
| 452. | —CH₂-A.82 |
| 453. | —CH₂-A.83 |
| 454. | —CH₂-A.84 |
| 455. | —CH₂-A.85 |
| 456. | —CH₂-A.86 |
| 457. | —CH₂-A.87 |
| 458. | —CH₂-A.88 |
| 459. | —CH₂-A.89 |
| 460. | —CH₂-A.90 |
| 461. | —CH₂-A.91 |
| 462. | —CH₂-A.92 |
| 463. | —CH₂-A.93 |
| 464. | —CH₂-A.94 |
| 465. | —CH₂-A.95 |
| 466. | —CH₂-A.96 |
| 467. | —CH₂-A.97 |
| 468. | —CH₂-A.98 |
| 469. | —CH₂-A.99 |
| 470. | —CH₂-A.100 |
| 471. | —CH₂-A.101 |
| 472. | —CH₂-A.102 |
| 473. | —CH₂-A.103 |
| 474. | —CH₂-A.104 |
| 475. | —CH₂-A.105 |
| 476. | —CH₂-A.106 |
| 477. | —CH₂-A.107 |
| 478. | —CH₂-A.108 |
| 479. | —CH₂-A.109 |
| 480. | —CH₂-A.110 |
| 481. | —CH₂-A.111 |
| 482. | —CH₂-A.112 |
| 483. | —CH₂-A.113 |
| 484. | —CH₂-A.114 |
| 485. | —CH₂-A.115 |
| 486. | —CH₂-A.116 |
| 487. | —CH₂-A.117 |
| 488. | —CH₂-A.118 |
| 489. | —CH₂-A.119 |
| 490. | —CH₂-A.120 |
| 491. | —CH₂-A.121 |
| 492. | —CH₂-A.122 |
| 493. | —CH₂-A.123 |
| 494. | —CH₂-A.124 |
| 495. | —CH₂-A.125 |
| 496. | —CH₂-A.126 |
| 497. | —CH₂-A.127 |
| 498. | —CH₂-A.128 |
| 499. | —CH₂-A.129 |
| 500. | —CH₂-A.130 |
| 501. | —CH₂-A.131 |
| 502. | —CH₂-A.132 |
| 503. | —CH₂-A.133 |
| 504. | —CH₂-A.134 |
| 505. | —CH₂-A.135 |
| 506. | —CH₂-A.136 |
| 507. | —CH₂-A.137 |
| 508. | —CH₂-A.138 |
| 509. | —CH₂-A.139 |
| 510. | —CH₂-A.140 |
| 511. | —CH₂-A.141 |
| 512. | —CH₂-A.142 |
| 513. | —CH₂-A.143 |
| 514. | —CH₂-A.144 |
| 515. | —CH₂-A.145 |
| 516. | —CH₂-A.146 |
| 517. | —CH₂-A.147 |
| 518. | —CH₂-A.148 |
| 519. | —CH₂-A.149 |
| 520. | —CH₂-A.150 |
| 521. | —CH₂CH₂-A.1 |
| 522. | —CH₂CH₂-A.2 |
| 523. | —CH₂CH₂-A.3 |
| 524. | —CH₂CH₂-A.4 |
| 525. | —CH₂CH₂-A.5 |
| 526. | —CH₂CH₂-A.6 |
| 527. | —CH₂CH₂-A.7 |
| 528. | —CH₂CH₂-A.8 |
| 529. | —CH₂CH₂-A.9 |
| 530.y | —CH₂CH₂-A.10 |
| 531. | —CH₂CH₂-A.11 |
| 532. | —CH₂CH₂-A.12 |
| 533. | —CH₂CH₂-A.13 |
| 534. | —CH₂CH₂-A.14 |
| 535. | —CH₂CH₂-A.15 |
| 536. | —CH₂CH₂-A.16 |
| 537. | —CH₂CH₂-A.17 |
| 538. | —CH₂CH₂-A.18 |
| 539. | —CH₂CH₂-A.19 |
| 540. | —CH₂CH₂-A.20 |
| 541. | —CH₂CH₂-A.21 |
| 542. | —CH₂CH₂-A.22 |
| 543. | —CH₂CH₂-A.23 |
| 544. | —CH₂CH₂-A.24 |
| 545. | —CH₂CH₂-A.25 |
| 546. | —CH₂CH₂-A.26 |
| 547. | —CH₂CH₂-A.27 |
| 548. | —CH₂CH₂-A.28 |
| 549. | —CH₂CH₂-A.29 |
| 550. | —CH₂CH₂-A.30 |
| 551. | —CH₂CH₂-A.31 |
| 552. | —CH₂CH₂-A.32 |
| 553. | —CH₂CH₂-A.33 |
| 554. | —CH₂CH₂-A.34 |
| 555. | —CH₂CH₂-A.35 |
| 556. | —CH₂CH₂-A.36 |
| 557. | —CH₂CH₂-A.37 |
| 558. | —CH₂CH₂-A.38 |
| 559. | —CH₂CH₂-A.39 |
| 560. | —CH₂CH₂-A.40 |
| 561. | —CH₂CH₂-A.41 |
| 562. | —CH₂CH₂-A.42 |
| 563. | —CH₂CH₂-A.43 |
| 564. | —CH₂CH₂-A.44 |
| 565. | —CH₂CH₂-A.45 |
| 566. | —CH₂CH₂-A.46 |
| 567. | —CH₂CH₂-A.47 |
| 568. | —CH₂CH₂-A.48 |
| 569. | —CH₂CH₂-A.49 |
| 570. | —CH₂CH₂-A.50 |
| 571. | —CH₂CH₂-A.51 |
| 572. | —CH₂CH₂-A.52 |
| 573. | —CH₂CH₂-A.53 |
| 574. | —CH₂CH₂-A.54 |
| 575. | —CH₂CH₂-A.55 |
| 576. | —CH₂CH₂-A.56 |
| 577. | —CH₂CH₂-A.57 |
| 578. | —CH₂CH₂-A.58 |
| 579. | —CH₂CH₂-A.59 |
| 580. | —CH₂CH₂-A.60 |
| 581. | —CH₂CH₂-A.61 |
| 582. | —CH₂CH₂-A.62 |
| 583. | —CH₂CH₂-A.63 |
| 584. | —CH₂CH₂-A.64 |
| 585. | —CH₂CH₂-A.65 |
| 586. | —CH₂CH₂-A.66 |
| 587. | —CH₂CH₂-A.67 |
| 588. | —CH₂CH₂-A.68 |
| 589. | —CH₂CH₂-A.69 |
| 590. | —CH₂CH₂-A.70 |
| 591. | —CH₂CH₂-A.71 |
| 592. | —CH₂CH₂-A.72 |
| 593. | —CH₂CH₂-A.73 |
| 594. | —CH₂CH₂-A.74 |
| 595. | —CH₂CH₂-A.75 |
| 596. | —CH₂CH₂-A.76 |
| 597. | —CH₂CH₂-A.77 |
| 598. | —CH₂CH₂-A.78 |
| 599. | —CH₂CH₂-A.79 |
| 600. | —CH₂CH₂-A.80 |
| 601. | —CH₂CH₂-A.81 |
| 602. | —CH₂CH₂-A.82 |
| 603. | —CH₂CH₂-A.83 |
| 604. | —CH₂CH₂-A.84 |
| 605. | —CH₂CH₂-A.85 |
| 606. | —CH₂CH₂-A.86 |
| 607. | —CH₂CH₂-A.87 |
| 608. | —CH₂CH₂-A.88 |
| 609. | —CH₂CH₂-A.89 |
| 610. | —CH₂CH₂-A.90 |
| 611. | —CH₂CH₂-A.91 |
| 612. | —CH₂CH₂-A.92 |
| 613. | —CH₂CH₂-A.93 |
| 614. | —CH₂CH₂-A.94 |
| 615. | —CH₂CH₂-A.95 |
| 616. | —CH₂CH₂-A.96 |
| 617. | —CH₂CH₂-A.97 |
| 618. | —CH₂CH₂-A.98 |
| 619. | —CH₂CH₂-A.99 |
| 620. | —CH₂CH₂-A.100 |
| 621. | —CH₂CH₂-A.101 |
| 622. | —CH₂CH₂-A.102 |
| 623. | —CH₂CH₂-A.103 |
| 624. | —CH₂CH₂-A.104 |
| 625. | —CH₂CH₂-A.105 |
| 626. | —CH₂CH₂-A.106 |
| 627. | —CH₂CH₂-A.107 |
| 628. | —CH₂CH₂-A.108 |
| 629. | —CH₂CH₂-A.109 |
| 630. | —CH₂CH₂-A.110 |
| 631. | —CH₂CH₂-A.111 |
| 632. | —CH₂CH₂-A.112 |
| 633. | —CH₂CH₂-A.113 |
| 634. | —CH₂CH₂-A.114 |
| 635. | —CH₂CH₂-A.115 |
| 636. | —CH₂CH₂-A.116 |
| 637. | —CH₂CH₂-A.117 |
| 638. | —CH₂CH₂-A.118 |
| 639. | —CH₂CH₂-A.119 |
| 640. | —CH₂CH₂-A.120 |
| 641. | —CH₂CH₂-A.121 |
| 642. | —CH₂CH₂-A.122 |
| 643. | —CH₂CH₂-A.123 |
| 644. | —CH₂CH₂-A.124 |
| 645. | —CH₂CH₂-A.125 |
| 646. | —CH₂CH₂-A.126 |
| 647. | —CH₂CH₂-A.127 |
| 648. | —CH₂CH₂-A.128 |
| 649. | —CH₂CH₂-A.129 |
| 650. | —CH₂CH₂-A.130 |
| 651. | —CH₂CH₂-A.131 |
| 652. | —CH₂CH₂-A.132 |
| 653. | —CH₂CH₂-A.133 |
| 654. | —CH₂CH₂-A.134 |
| 655. | —CH₂CH₂-A.135 |
| 656. | —CH₂CH₂-A.136 |
| 657. | —CH₂CH₂-A.137 |
| 658. | —CH₂CH₂-A.138 |
| 659. | —CH₂CH₂-A.139 |
| 660. | —CH₂CH₂-A.140 |
| 661. | —CH₂CH₂-A.141 |
| 662. | —CH₂CH₂-A.142 |
| 663. | —CH₂CH₂-A.143 |
| 664. | —CH₂CH₂-A.144 |
| 665. | —CH₂CH₂-A.145 |
| 666. | —CH₂CH₂-A.146 |
| 667. | —CH₂CH₂-A.147 |
| 668. | —CH₂CH₂-A.148 |
| 669. | —CH₂CH₂-A.149 |
| 670. | —CH₂CH₂-A.150 |

A. 1 to A. 150 are the rings depicted below, where # is the attachment point to O:

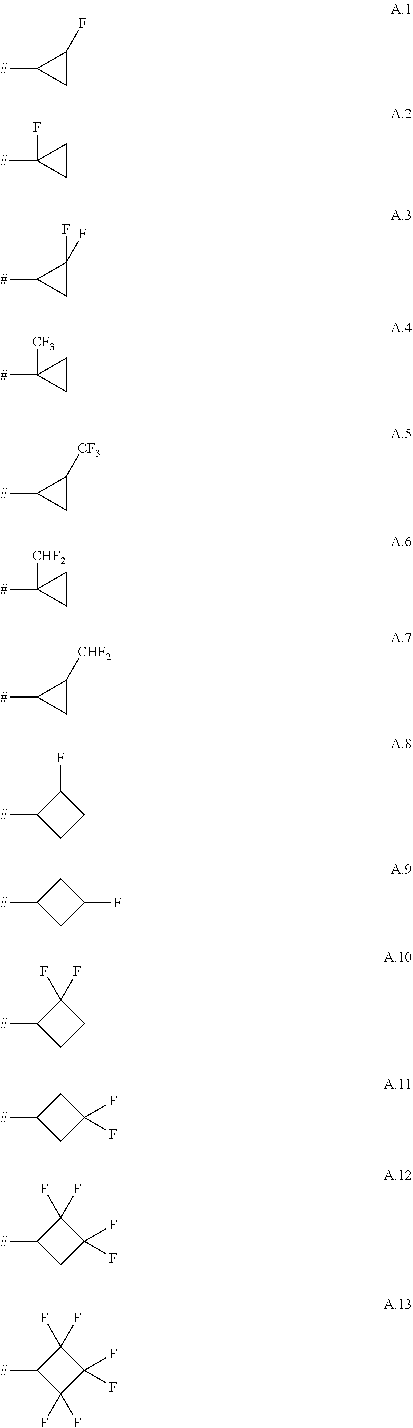

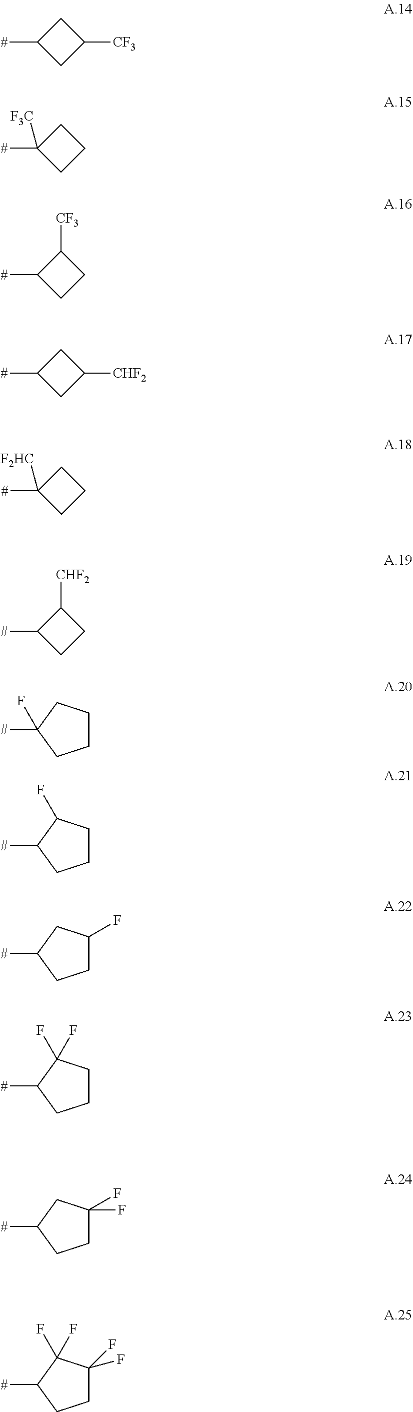

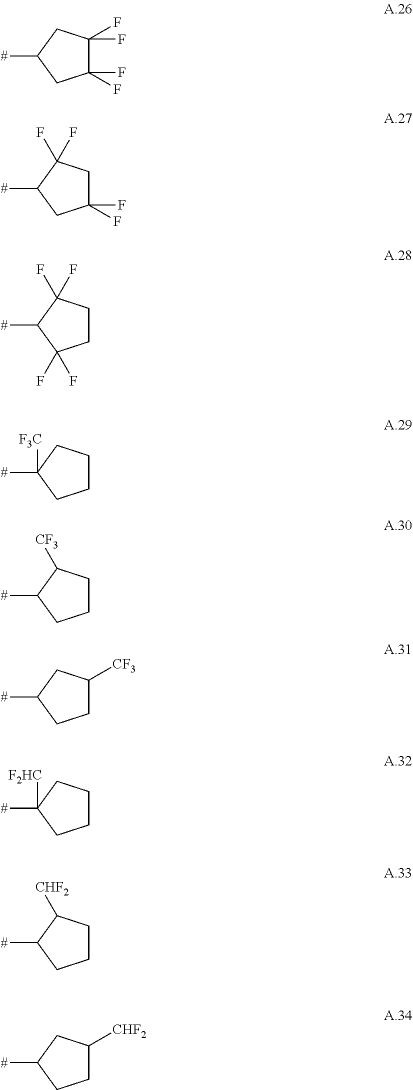

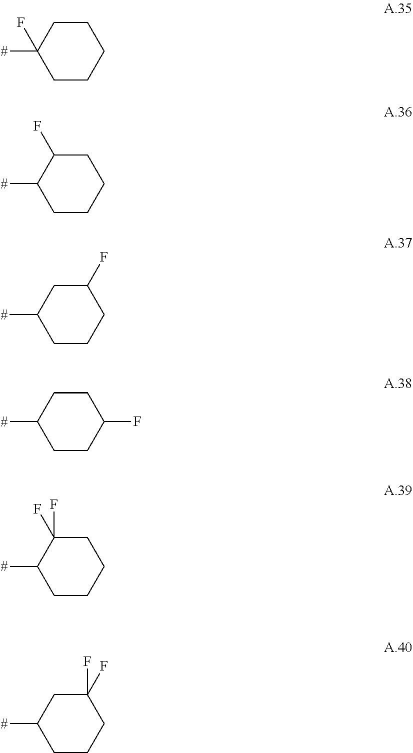

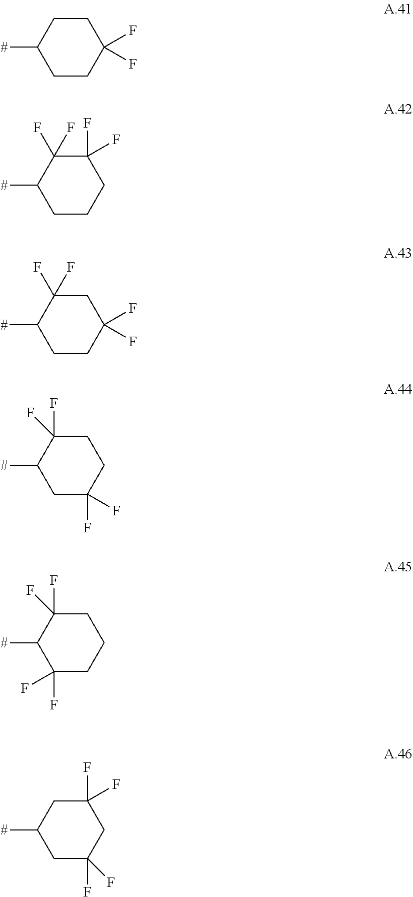

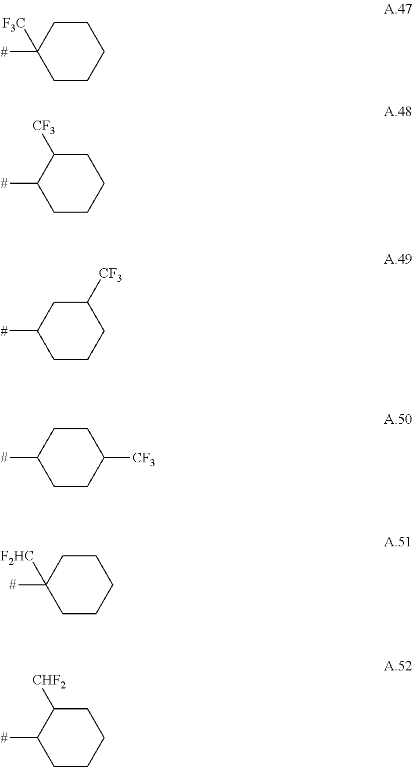

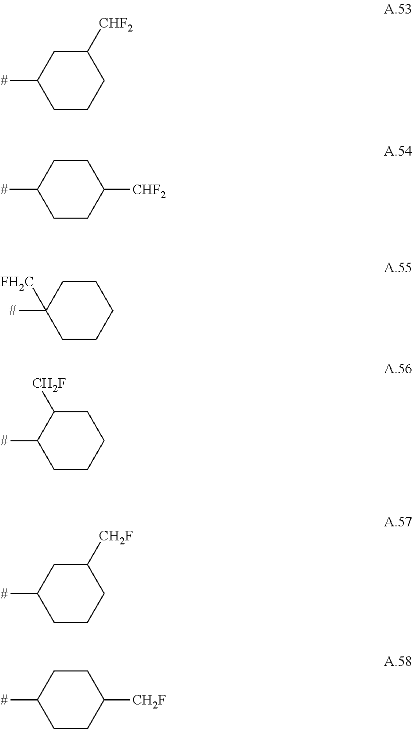

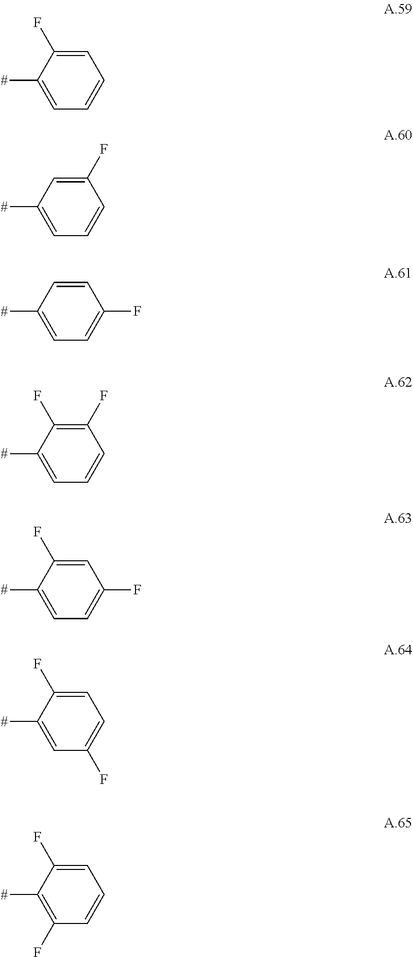

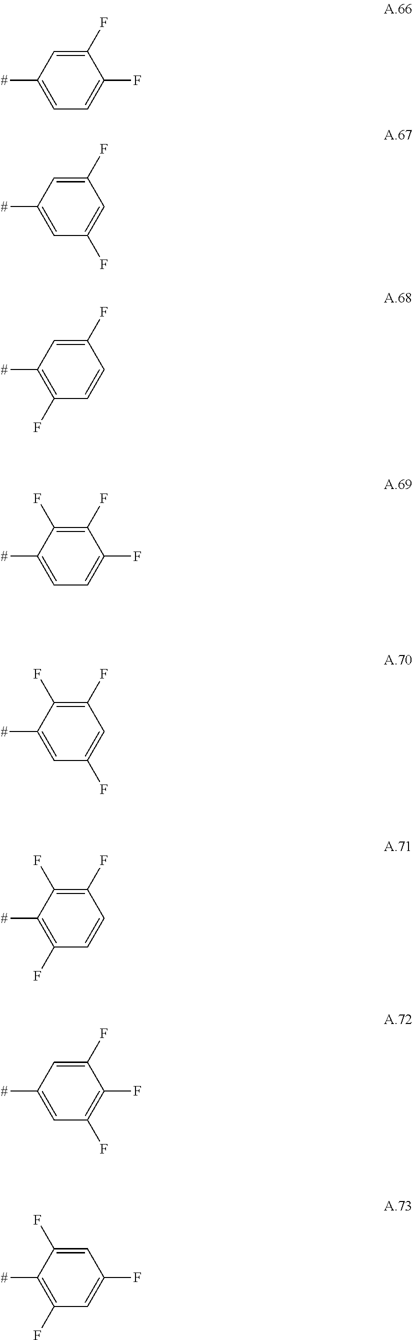

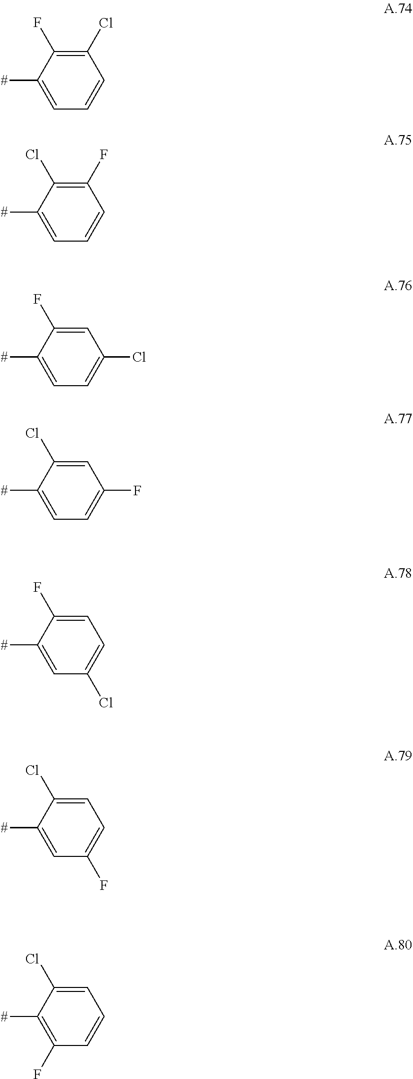

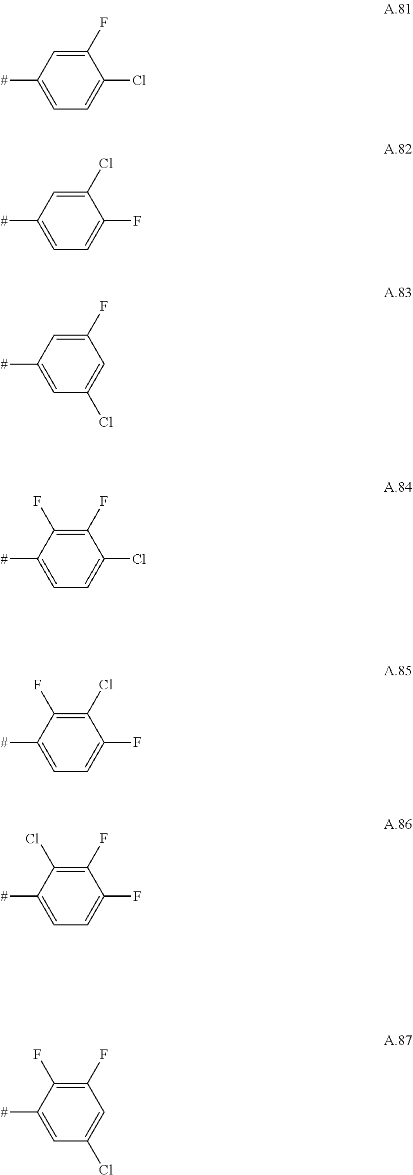

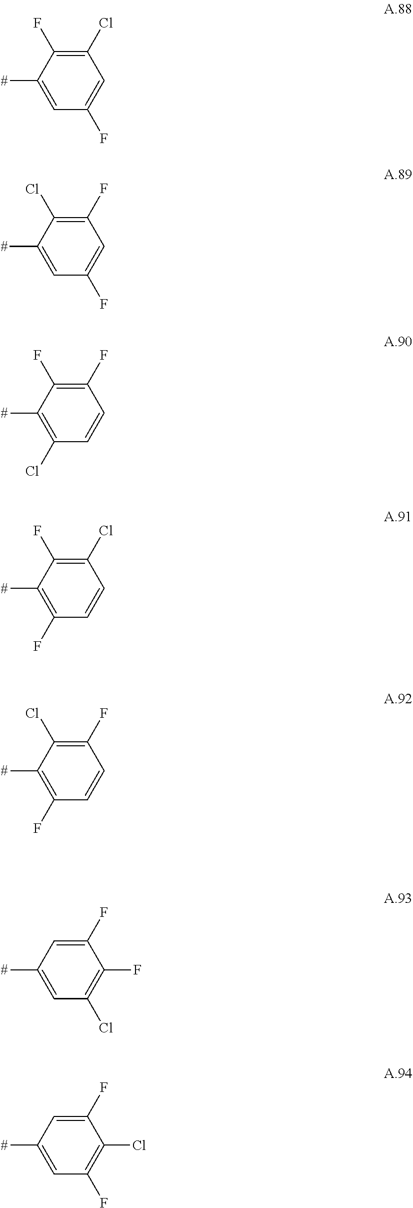

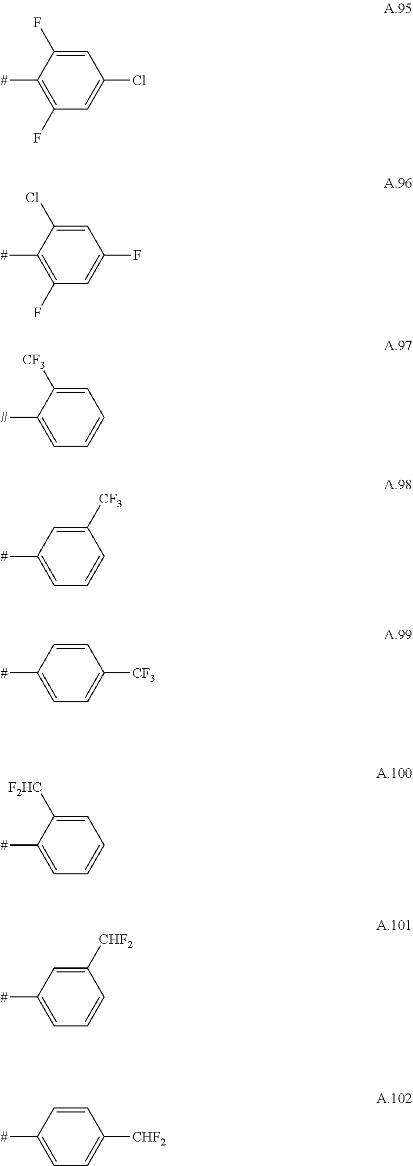

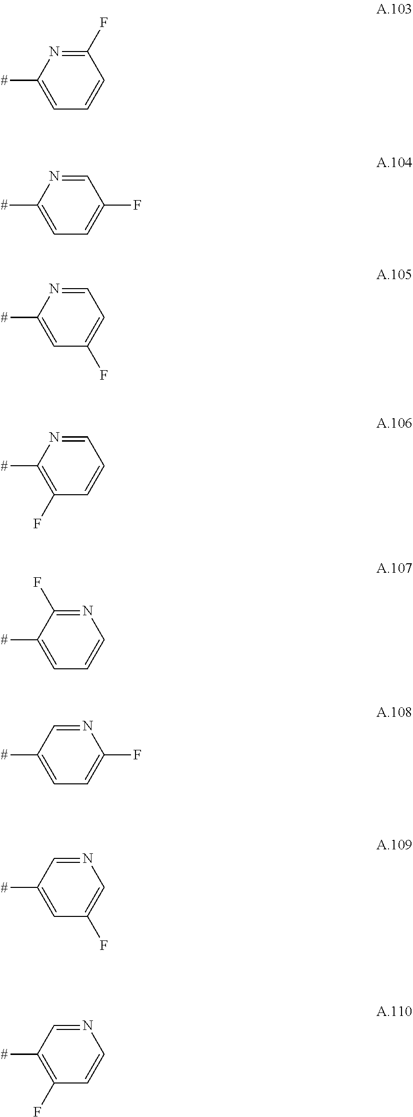

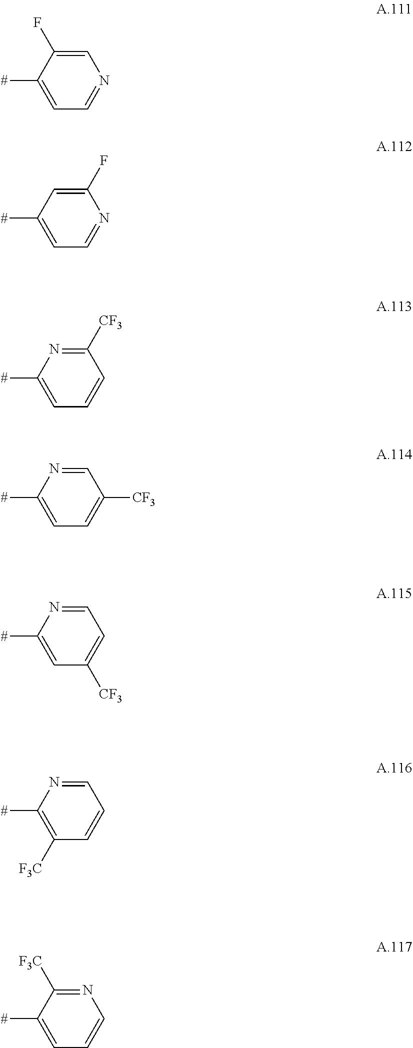

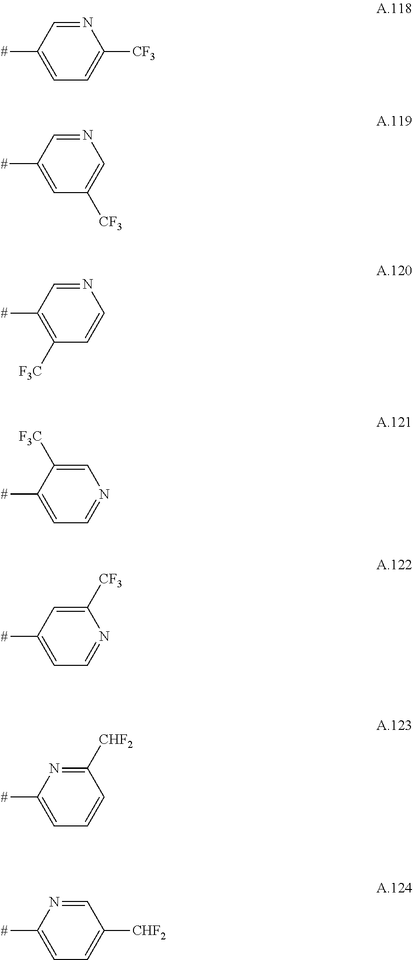

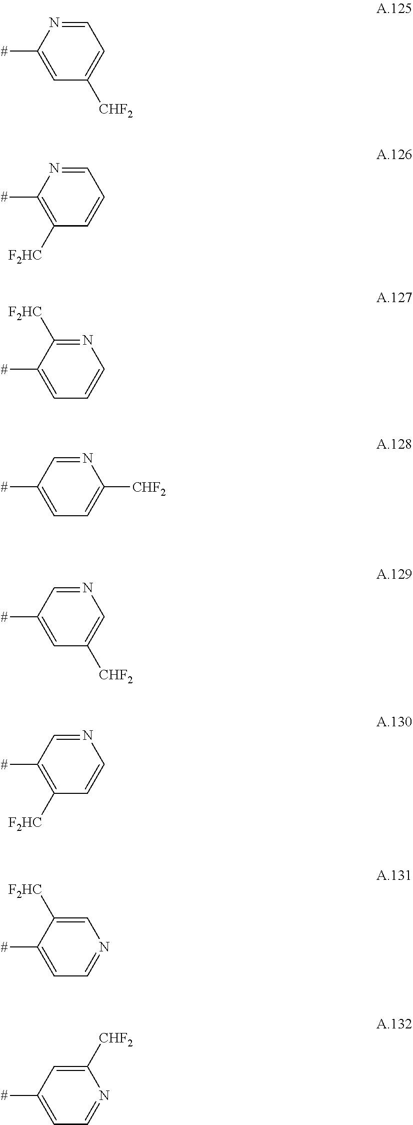

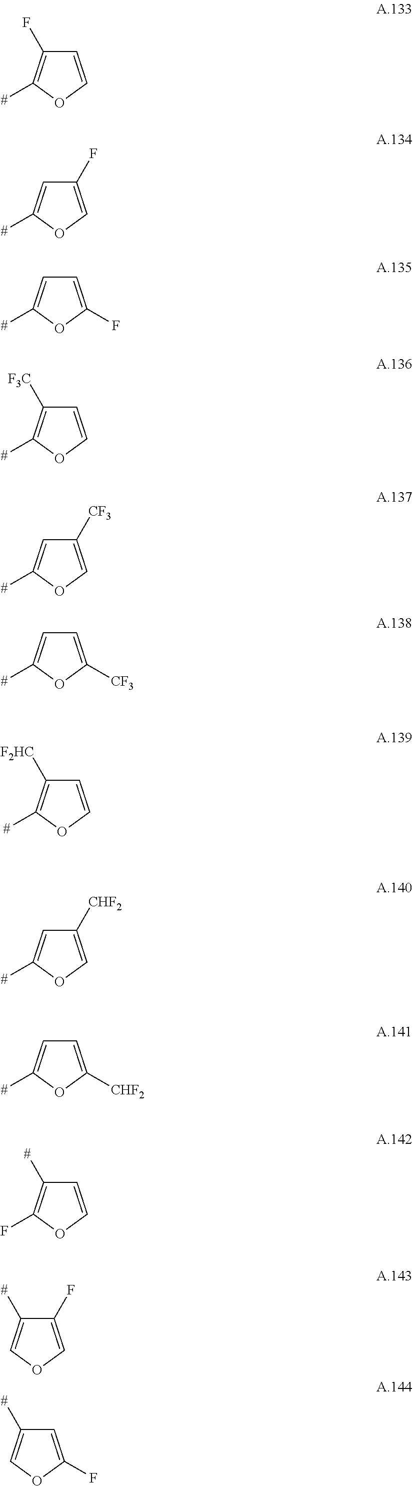

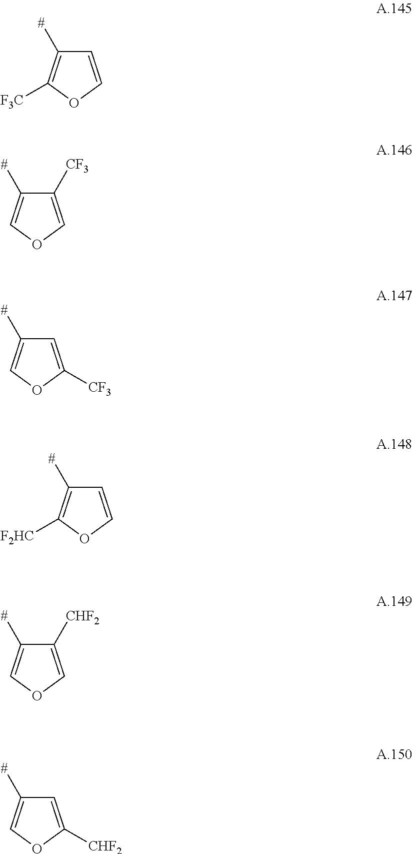

Among the above structures Ia.1 to Ia.6, preference is given to compounds Ia.1.

In a specific embodiment, the invention relates to compounds I selected from the compounds of the examples, either in form of free bases or of any pharmaceutically acceptable salt thereof or a stereoisomer, the racemate or any mixture of stereoisomers thereof or a tautomer or a tautomeric mixture or an N-oxide thereof.

The compounds of the present invention can be prepared by using routine techniques familiar to a skilled person. In particular, the compounds of the formula I can be prepared according to the following schemes, wherein the variables, if not stated otherwise, are as defined above.

Compounds of formula I or precursors thereof wherein the group R³ or the group OR³ is not yet present (called hereinafter compounds I′) can be prepared as outlined in scheme 1 below. Amine 1 is coupled with the pyrrolidine or azetidine acid derivative 2 under standard amidation conditions, wherein LG represents a suitable leaving group, such as Cl, Br, I or a sulfonate, such as tosylate, mesylate, triflate or nonaflate. The reaction is generally carried out under basic conditions. Alternatively, LG is OH and amidation is carried out in the presence of a coupling reagent. Suitable coupling reagents (activators) are well known and are for instance selected from carbodiimides, such as DCC (dicyclohexylcarbodiimide), DCI (diisopropylcarbodiimide) and EDCI (1-ethyl-3-(3-dimethylaminopropyl)carbodiimide), benzotriazol derivatives, such as HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate). Generally, the activator is used in excess. The benzotriazol and phosphonium coupling reagents are generally used in a basic medium.

R stands for OR³, OR^{3a} or OH, where R^{3a} is a precursor of R³. R′ stands for a protective group or for CH₃.

Suitable protective groups are for example C₁-C₄-alkylcarbonyl (e.g. acetyl), C₁-C₄-haloalkylcarbonyl (e.g. trifluoroacetyl), C₃-C₄-alkenylcarbonyl (e.g. allylcarbonyl), C₁-C₄-alkoxycarbonyl (e.g. Boc), C₁-C₄-haloalkoxycarbonyl, C₃-C₄-alkenyloxycarbonyl, C₁-C₄-alkylaminocarbonyl, di-(C₁-C₄-alkyl)-aminocarbonyl, C₁-C₄-alkyl-sulfonyl, C₁-C₄-haloalkylsulfonyl or benzyl. The choice of the protective group depends on the reaction conditions in the amidation reaction. The protective group is chosen so that it is not hydrolyzed during the amidation reaction.

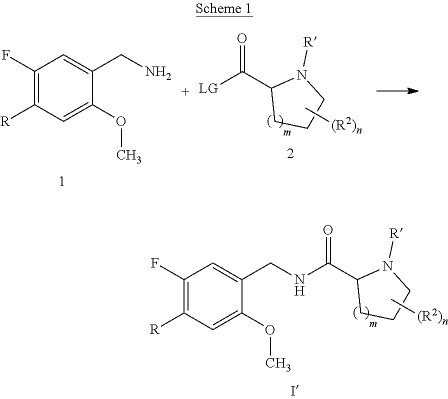

If R is OR³ and R′ is a protective group, compound I′ is deprotected to give compounds I. Deprotection conditions depend on the protective group used.

If R is OR^{3a} and R′ is a protective group, the group R^{3a} is generally first converted into the group R³ before the protective group is removed. Analogously, if R is OH, generally R is first converted into the group OR³ before the protective group is removed.

The conversion of compounds I′ wherein R is OH (called hereinafter compounds 4) into compounds I′ wherein R is OR³ or OR^{3a} (called hereinafter compounds I″) can be carried out as outlined in scheme 2 below. The compound 3 and the hydroxyphenyl compound 4 are reacted under ether formation to I″. R^{3b} is R³ or a precursor R^{3a} of R³. LG is a leaving group, such as Cl, Br, I or a sulfonate, e.g. tosylate, mesylate, triflate or nonaflate. The reaction is generally carried out under basic conditions.

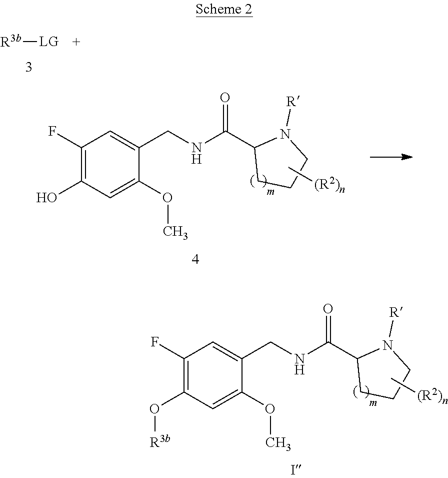

Alternatively, the conversion of compounds I′ wherein R is OH (called hereinafter compounds 4) into compounds I′ wherein R is OR³ or OR^{3a} (called hereinafter compounds I″) can be carried out as outlined in scheme 3 below. The two hydroxy compounds 4 and 5 are reacted under Mitsunobu conditions to I″ using triphenylphosphine and an azodicarboxylate such as diethyl azodicarboxylate (DEAD), diisopropyl azodicarboxylate (DIAD), di-tert-butylazodicarboxylate or di-(4-chlorobenzyl)azodicarboxylate (DCAD). Instead of triphenylphosphine, resin-bound triphenylphoshine, such as PS—PPh₃ (resin=crosslinked poly(styrene-codivinylbenzene), can be used. R^{3b} is R³ or a precursor R^{3a} of R³.

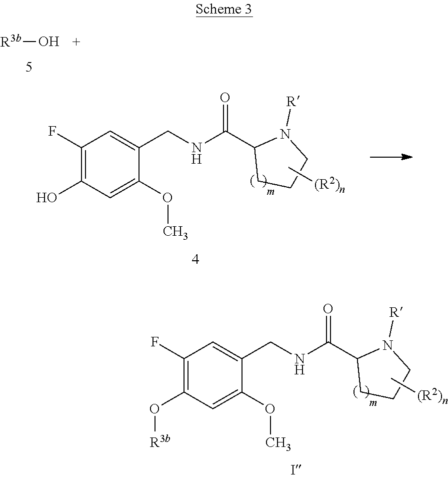

Compounds 1 can be prepared by reduction of the corresponding benzonitrile 6, as depicted in scheme 4 below. Suitable reduction agents are hydrogen (generally in form of a catalytic hydrogenation using, e.g. Pd/C or Ni, e.g. in form of Raney Ni), complex hydrides, such as sodium boron hydride (NaBH₄), sodium boron hydride (NaBH₄)/cobald-II-cloride, lithium triethylborohydride (superhydride; LiBH(CH₂CH₃)₂), lithium tri-sec-butyl(hydrido)borate (L-selectride; LiBH(CH(CH₃)CH₂CH₃)₂), lithium aluminum hydride (LAH; LiAlH₄) or diisobutlyaluminum hydride (DIBAL-H; ((CH₃)₂CHCH₂)₂AlH), or boranes, e.g. diborane or borane complexes, such as borane-dimethylsulfide complex, borane-diethylether complex or borane-THF complex.

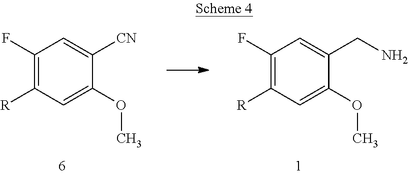

Compounds 6, wherein R is OR³ or OR^{3a} (called hereinafter compounds 6′), can be prepared in analogy to the reactions depicted in schemes 2 and 3 by reacting compounds 6, wherein R is OH (called hereinafter compounds 7), with 3 or the alcohol 5. Suitable conditions for the respective reactions correspond to those detailed above in context with schemes 2 and 3. Alternatively, compounds 6′ can be prepared starting from compounds 6 wherein R is F (called hereinafter compounds 8). The conversion of compounds 8 into compounds 6′ is carried out under basic conditions. Generally, the alcohol 5 is first deprotonated using strong, non-nucleophilic bases, such as NaH or potassium tert-butanolate, before 8 is added. The above reactions are outlined in scheme 5 below. R^{3b} is R³ or a precursor R^{3a} of R³. LG is a leaving group, such as Cl, Br, I or a sulfonate, e.g. tosylate, mesylate, triflate or nonaflate.

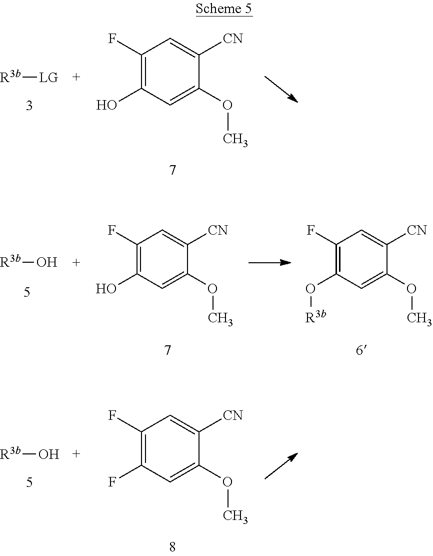

Alternatively, compounds 6′ can be prepared starting from the trifluoro compound 9, as shown in scheme 6 below. Due to the para-directing effect of CN the regioselectivity (as compared to the substitution of the fluorine substituent in ortho-position to CN and to the substitution of both fluorine atoms by —OR^{3b}) is high if 9 and 5 are used in approximately stoichiometric amounts. Use of 5 in excess yields mixtures of the two regioisomers as well as compounds in which both fluorine atoms are replaced by —OR^{3b}In this case 10 has to be separated from the undesired side products by usual means, such as chromatography etc.). The reaction of compounds 9 with 5 is generally carried out under basic conditions. Generally, the alcohol 5 is first deprotonated using strong, non-nucleophilic bases, such as NaH or potassium tert-butanolate, before 9 is added. Subsequent reaction of 10 with methanol, also generally under basic conditions, with methanol being generally first deprotonated using strong, non-nucleophilic bases, such as NaH or potassium tert-butanolate, before 10 is added, yields 6′.

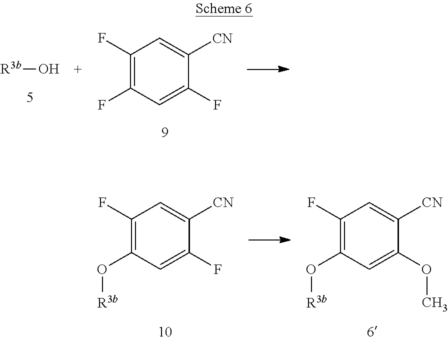

Alternatively, compounds 6′ can be prepared by a Buchwald-Hartwig-analogous Pd coupling of 11 with the alcohol 5. The Pd catalyst is usually used with a phosphorus ligand, such as 2,2′-bis(diphenylphosphino)-1,1′-binaphthyl (BINAP), [1,1′-biphenyl]-2-diisopropyl phosphine, 1,1′-bis(diphenylphospino)ferrocene (dppf), X-phos, di-tert-butyl(2′,4′,6′-triisopropyl-[1,1′-biphenyl]-2-yl)phosphine (t-BuXPhos), 9,9-dimethyl-4,5-bis(diphenylphosphino)xanthene (Xantphos), 4,5-bis-(di-1-(3-methylindolyl)-phosphoramidit)-2,7,9,9-tetramethylxanthene (MeSkatOX), triphenylphosphine, triphenylphosphite, tri-(2-(1,1-dimethylethyl)-4-methoxy-phenyl)-phosphite, tricyclohexylphosphine, butyldi-1-adamantylphosphine (cataCXium), 1,6-bis(diphenylphosphino)-hexane (DPPH), 2,6-bis(2,5-dimethylphenyl)-1-octyl-4-phenylphosphacyclohexan (PCH), 2-dicyclohexylphosphino-2′,6′-diisopropoxybiphenyl (RuPhos) and the like. The reaction is generally carried out in the presence of a base, advantageously a non-nucleophilic base, e.g. a carbonate, such as lithium, sodium, potassium or caesium carbonate, DBU, DBN and the like, or a sterically hindered nucleophilic alcoholate, like sodium or potassium tert-butanolate. Sterically non-demanding nucleophilic bases can be used if they are first reacted with the alcohol 5 before compound 11 is added. Suitable bases for this purpose are e.g. methanolates, e.g. sodium or potassium methanolate, ethanolates, e.g. sodium or potassium ethanolate, hydroxides, such as sodium or potassium hydroxide, hydrides, such as sodium or potassium hydride, and LDA. Non-nucleophilic bases or sterically hindered nucleophilic alcoholates can of course also be used for first deprotonating the alcohol 5 before compound 11 is added, as long as they are strong enough for the deprotonation.

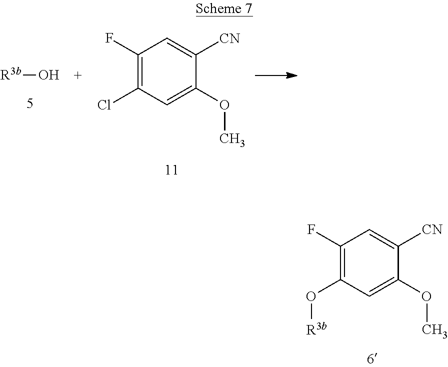

The above-described Buchwald-Hartwig coupling can also be applied in a downstream reaction step, as depicted in scheme 8, to obtain compounds I″. The reaction conditions correspond to those described above in context with scheme 7. R^{3b} is R³ or a precursor R^{3a} of R³.

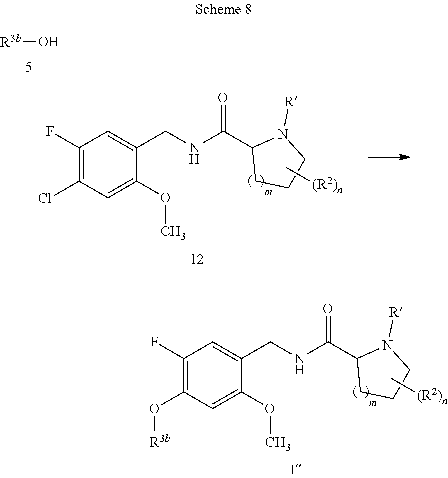

Compounds 12 can be prepared in analogy to scheme 1.

In the above reactions, precursor groups R^{3a} are expediently used instead of the final groups R³ if the desired group R³ is susceptible to the reaction conditions or can compete in one of the required reaction steps. An example for such a group is a radical CH₂F. In this case, it is expedient, for example, to use the respective alcohol precursor group CH₂—OH, suitably in protected form, and convert the alcohol into the CH₂F group only at end of the reaction (deprotection, if R′ is a protective group, can however ensue). Suitable protective groups are known and are generally selected from silyl protective groups, such as TMS (trimethylsilyl), TES (triethylsilyl, TBDMS (tert-butyldimethylsilyl), TIPS (triisopropylsilyl) or TBDPS (tert-butyldiphenylsilyl). As the OH group is not very reactive towards fluorination agents, this is suitably first converted into a better leaving group, e.g. by reaction to a sulfonate, such as methylsulfonate, tolylsulfonate and the like. This is then reacted with a fluorination agent, such as an alkali metal fluoride, e.g. NaF, KF or CsF; HF, optionally in combination with SbCl₅ or with Cl₂ and SbF₃; SF₄, optionally in combination with HF or BF₃[O(C₂H₅)₂]; phenylsulfur trifluoride (Ph-SF₃), optionally in combination with HF and pyridine; 4-tert-butyl-2,6-dimethylphenyl sulfur trifluoride (“Fluoled”); and bis(2-methoxyethyl)amino sulfur trifluoride [(CH₃OCH₂CH₂)₂NSF₃].

If not otherwise indicated, the above-described reactions are generally carried out in a solvent at temperatures between room temperature and the boiling temperature of the solvent employed. Alternatively, the activation energy which is required for the reaction can be introduced into the reaction mixture using microwaves, something which has proved to be of value, in particular, in the case of the reactions catalyzed by transition metals (with regard to reactions using microwaves, see Tetrahedron 2001, 57, p. 9199 ff. p. 9225 ff. and also, in a general manner, “Microwaves in Organic Synthesis”, André Loupy (Ed.), Wiley-VCH 2002).

The acid addition salts of compounds I are prepared in a customary manner by mixing the free base with a corresponding acid, where appropriate in solution in an organic solvent, for example a lower alcohol, such as methanol, ethanol or propanol, an ether, such as methyl tert-butyl ether or diisopropyl ether, a ketone, such as acetone or methyl ethyl ketone, or an ester, such as ethyl acetate.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the preparation methods are within routine techniques.

Suitable protecting groups and the methods for protecting and deprotecting different substituents using such suitable protecting groups are well known to those skilled in the art; examples of which may be found in T. Greene and P. Wuts, Protective Groups in Organic Synthesis (3^{rd} ed.), John Wiley & Sons, NY (1999), which is herein incorporated by reference in its entirety. Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it may be obtained by carrying out one of the above procedures using a pure geometric isomer as a starting material, or by resolution of a mixture of the geometric isomers of the compound or intermediates using a standard procedure such as chromatographic separation.

The present invention moreover relates to compounds of formula I as defined above, wherein at least one hydrogen atom has been replaced by a deuterium atom.

Of course, the unlabeled compounds according to the invention might naturally include certain amounts of this isotope. Therefore, when referring to compounds I, wherein at least one of the hydrogen atoms has been replaced by deuterium, it will be understood that the D isotope is present in a higher amount than would naturally occur.

Deuterated compounds have been used in pharmaceutical research to investigate the in vivo metabolic fate of the compounds by evaluation of the mechanism of action and metabolic pathway of the non deuterated parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

Substitution of deuterium for hydrogen can give rise to an isotope effect that could alter the pharmacokinetics of the drug.

Stable isotope labeling of a drug can alter its physico-chemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance. These effects and alterations can affect the pharmacodynamic response of the drug molecule if the isotopic substitution affects a region involved in a ligand-receptor interaction.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to “kinetic isotope effect”. A reaction involving breaking a C-D bond can be up to 700 percent slower than a similar reaction involving breaking a C—H bond. If the C-D bond is not involved in any of the steps leading to the metabolite, there may not be any effect to alter the behavior of the drug. If a deuterium is placed at a site involved in the metabolism of a drug, an isotope effect will be observed only if breaking of the C-D bond is the rate limiting step. There is evidence to suggest that whenever cleavage of an aliphatic C—H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway a process called “metabolic switching”.

Deuterium tracers, such as deuterium-labeled drugs and doses, in some cases repeatedly, of thousands of milligrams of deuterated water, are also used in healthy humans of all ages, including neonates and pregnant women, without reported incident (e.g. Pons G and Rey E, Pediatrics 1999 104: 633; Coward W A et al., Lancet 1979 7: 13; Schwarcz H P, Control. Clin. Trials 1984 5(4 Suppl): 573; Rodewald L E et al., J. Pediatr. 1989 114: 885; Butte N F et al. Br. J. Nutr. 1991 65: 3; MacLennan A H et al. Am. J. Obstet Gynecol. 1981 139: 948). Thus, it is clear that any deuterium released, for instance, during the metabolism of compounds of this invention poses no health risk.

The weight percentage of hydrogen in a mammal (approximately 9%) and natural abundance of deuterium (approximately 0.015%) indicates that a 70 kg human normally contains nearly a gram of deuterium. Furthermore, replacement of up to about 15% of normal hydrogen with deuterium has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Higher deuterium concentrations, usually in excess of 20%, can be toxic in animals. However, acute replacement of as high as 15%-23% of the hydrogen in humans' fluids with deuterium was found not to cause toxicity (Blagojevic N et al. in “Dosimetry & Treatment Planning for Neutron Capture Therapy”, ZamenhofR, Solares G and Harling O Eds. 1994. Advanced Medical Publishing, Madison Wis. pp. 125-134; Diabetes Metab. 23: 251 (1997)).

Increasing the amount of deuterium present in a compound above its natural abundance is called enrichment or deuterium-enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

The hydrogens present on a particular organic compound have different capacities for exchange with deuterium. Certain hydrogen atoms are easily exchangeable under physiological conditions and, if replaced by deuterium atoms, it is expected that they will readily exchange for protons after administration to a patient. Certain hydrogen atoms may be exchanged for deuterium atoms by the action of a deuteric acid such as D2SO4/D2O. Alternatively, deuterium atoms may be incorporated in various combinations during the synthesis of compounds of the invention. Certain hydrogen atoms are not easily exchangeable for deuterium atoms. However, deuterium atoms at the remaining positions may be incorporated by the use of deuterated starting materials or intermediates during the construction of compounds of the invention.

Deuterated and deuterium-enriched compounds of the invention can be prepared by using known methods described in the literature. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO 1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; 20090082471, the methods are hereby incorporated by reference.

The present invention further relates to a pharmaceutical composition comprising a therapeutically effective amount of at least one compound I as defined above or an N-oxide, a tautomeric form, a stereoisomer or a pharmaceutically acceptable salt thereof, in combination with at least one pharmaceutically acceptable carrier and/or auxiliary substance; or comprising at least one compound I wherein at least one of the atoms has been replaced by its stable, non-radioactive isotope, preferably wherein at least one hydrogen atom has been replaced by a deuterium atom, in combination with at least one pharmaceutically acceptable carrier and/or auxiliary substance.

The present invention further relates to a compound I as defined above or an N-oxide, a tautomeric form, a stereoisomer or a pharmaceutically acceptable salt thereof for use as a medicament.

The present invention also relates to a compound I as defined above or an N-oxide, a tautomeric form, a stereoisomer or a pharmaceutically acceptable salt thereof for use in the treatment of disorders which respond to the modulation of the 5-HT₂c receptor.

The present invention also relates to the use of a compound I as defined above or of an N-oxide, a tautomeric form, a stereoisomer or a pharmaceutically acceptable salt thereof for the manufacture of a medicament for the treatment of disorders which respond to the modulation of the 5-HT_{2C} receptor, and to a method for treating disorders which respond to the modulation of the 5-HT_{2C} receptor, which method comprises administering to a subject in need thereof at least one compound I as defined above or an N-oxide, a tautomeric form, a stereoisomer or a pharmaceutically acceptable salt thereof.

The compounds of the present invention are modulators of the 5-HT_{2C} receptor. Specifically, the compounds of formula I are agonists or partial agonists of the 5-HT_{2C} receptor. Thus, in a specific embodiment, the invention relates to a compound I as defined above or an N-oxide, a tautomeric form, a stereoisomer or a pharmaceutically acceptable salt thereof for the treatment of disorders which respond to 5-HT_{2C} receptor agonists, further to the use of a compound I as defined above or of an N-oxide, a tautomeric form, a stereoisomer or a pharmaceutically acceptable salt thereof for the manufacture of a medicament for the treatment of disorders which respond to 5-HT_{2C} receptor agonists, and to a method for treating disorders which respond to 5-HT_{2C} receptor agonists, which method comprises administering to a subject in need thereof at least one compound I as defined above or an N-oxide, a tautomeric form, a stereoisomer or a pharmaceutically acceptable salt thereof.

Within the meaning of the invention, the term “disorder” denotes disturbances and/or anomalies which are as a rule regarded as being pathological conditions or functions and which can manifest themselves in the form of particular signs, symptoms and/or malfunctions. While the treatment according to the invention can be directed toward individual disorders, i.e. anomalies or pathological conditions, it is also possible for several anomalies, which may be causatively linked to each other, to be combined into patterns, i.e. syndromes, which can be treated in accordance with the invention.

In one aspect of the invention, the diseases to be treated are disorders are damage of the central nervous system, disorders of the central nervous system, eating disorders, ocular hypertension, cardiovascular disorders, gastrointestinal disorders and diabetes.

Disorders or diseases of the central nervous system are understood as meaning disorders which affect the spinal cord and, in particular, the brain. These are, for example, cognitive dysfunction, attention deficit disorder/hyperactivity syndrome and cognitive deficits related with schizophrenia, attention deficit/hyperactivity syndrome, personality disorders, affective disorders, motion or motor disorders, pain, migraine, sleep disorders (including disturbances of the Circadian rhythm), feeding disorders, diseases associated with neurodegeneration, addiction diseases, obesity or psoriasis.

Examples of cognitive dysfunction are deficits in memory, cognition, and learning, Alzheimer's disease, age-related cognitive decline, and mild cognitive impairment, or any combinations thereof. Examples of personality disorders are schizophrenia and cognitive deficits related to schizophrenia. Examples of affective disorders are depression, anxiety, bipolar disorder and obsessive compulsive disorders, or any combination thereof. Examples of motion or motor disorders are Parkinson's disease and epilepsy. Examples of feeding disorders are obesity, bulimia, weight loss and anorexia, especially anorexia nervosa. Examples of diseases associated with neurodegeneration are stroke, spinal or head trauma, and head injuries, such as hydrocephalus.

Pain condition includes nociceptive pain, neuropathic pain or a combination thereof. Such pain conditions or disorders can include, but are not limited to, post-operative pain, osteoarthritis pain, pain due to inflammation, rheumatoid arthritis pain, musculoskeletal pain, burn pain (including sunburn), ocular pain, the pain associated with dental conditions (such as dental caries and gingivitis), post-partum pain, bone fracture, herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, hyperalgesia and cancer.

In certain other embodiments, the disease condition is bladder dysfunction, including urinary incontinence.

Diabetes includes diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

The addiction diseases include psychiatric disorders and behavioral disturbances which are caused by the abuse of psychotropic substances, such as pharmaceuticals or narcotics, and also other addiction diseases, such as addiction to gaming (impulse control disorders not elsewhere classified). Examples of addictive substances are: opioids (e.g. morphine, heroin and codeine), cocaine; nicotine; alcohol; substances which interact with the GABA chloride channel complex, sedatives, hypnotics and tranquilizers, for example benzodiazepines; LSD; cannabinoids; psychomotor stimulants, such as 3,4-methylenedioxy-N-methylamphetamine (ecstasy); amphetamine and amphetamine-like substances such as methylphenidate, other stimulants including caffeine and nicotine. Addictive substances which come particularly into consideration are opioids, cocaine, amphetamine or amphetamine-like substances, nicotine and alcohol. Especially, addiction disorders include alcohol abuse, cocaine abuse, tobacco abuse and smoking cessation.

With regard to the treatment of addiction diseases, particular preference is given to those compounds according to the invention of the formula (I) which themselves do not possess any psychotropic effect. This can also be observed in a test using rats, which, after having been administered compounds which can be used in accordance with the invention, reduce their self administration of psychotropic substances, for example cocaine.

Examples of gastrointestinal disorders are irritable bowel syndrome.

Preferably, the disorders are selected from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, psychosis in Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance, and are specifically schizophrenia, depression, bipolar disorders, obesity, substance use disorders, neuropsychiatric symptoms in Alzheimer's disease (e.g. aggression) or neuropsychiatric symptoms in Parkinson's disease (e.g. aggression).

The compounds of the invention may be used for a preventive treatment (prophylaxis), in particular as relapse prophylaxis or phase prophylaxis, but are preferably used for a treatment in its proper sense (i.e. non-prophylactic), i.e. for the treatment of acute or chronic signs, symptoms and/or malfunctions. The treatment can be orientated symptomatically, for example as the suppression of symptoms. It can be effected over a short period, be orientated over the medium term or can be a long-term treatment, for example within the context of a maintenance therapy.

In another embodiment, the present invention relates to the use of a compound I as defined above or an N-oxide, a tautomeric form, a stereoisomer or a pharmaceutically acceptable salt thereof for preparing a medicament for preventing (the development of) a disease condition as described above and to a method for preventing (the development of) a disease condition as described above comprises administering to the subject in need of treatment thereof (e.g., a mammal, such as a human) a therapeutically effective amount of a compound I as defined above or an N-oxide, a tautomeric form, a stereoisomer or a pharmaceutically acceptable salt thereof. As used herein, the term “prevent” a disease condition by administration of any of the compounds described herein means that the detectable physical characteristics or symptoms of the disease or condition do not develop following the administration of the compound described herein. Alternatively, the method comprises administering to the subject a therapeutically effective amount of a compound I as defined above or an N-oxide, a tautomeric form, a stereoisomer or a pharmaceutically acceptable salt thereof, in combination with a therapeutically effective amount of at least one cognitive enhancing drug.

In yet another embodiment, the present invention relates to the use a compound I as defined above or an N-oxide, a tautomeric form, a stereoisomer or a pharmaceutically acceptable salt thereof for preparing a medicament for preventing the progression (e.g., worsening) of a disease condition and to a method for preventing the progression (e.g., worsening) of a disease condition, which method comprises administering to the subject in need of treatment thereof (e.g., a mammal, such as a human) a therapeutically effective amount of a compound I as defined above or an N-oxide, a tautomeric form, a stereoisomer or a pharmaceutically acceptable salt thereof.

There are several lines of evidence suggesting that 5-HT_{2C} agonists or partial agonists would have therapeutic use in a variety of diseases, disorders and conditions.

Knockout mice models lacking the 5-HT_{2C} receptor exhibit hyperphagia, obesity and are more prone to seizures and sudden death [Tecott L H, Sun L M, Akana S F, Strack A M, Lowenstein D H, Dallman M F, Julius D (1995) Eating disorder and epilepsy in mice lacking 5-HT_{2C} serotonin receptors. Nature 374:542-546]. They also exhibit compulsive-like behavior [Chou-Green J M, Holscher T D, Dallman M F, Akana S F (2003). Compulsive behavior in the 5-HT_{2C} receptor knockout mouse. Phys. Behav. 78:641-649], hyperresponsiveness to repeated stress [Chou-Green J M, Holscher T D, Dallman M F, Akana S F (2003). Repeated stress in young and old 5-HT_{2C} receptor knockout mouse. Phys. Behav. 79:217-226], wakefulness [Frank M G, Stryker M P, Tecott L H (2002). Sleep and sleep homeostasis in mice lacking the 5-HT_{2C} receptor. Neuropsychopharmacology 27:869-873], hyperactivity and drug dependence [Rocha B A, Goulding E H, O'Dell L E, Mead A N, Coufal N G, Parsons L H, Tecott L H (2002). Enhanced locomotor, reinforcing and neurochemical effects of cocaine in serotonin 5-hydroxytryptamine 2C receptor mutant mice. J. Neurosci. 22:10039-10045].

5-HT_{2C} is unique among other G-protein-coupled receptors (GPCRs) in that its pre-mRNA is a substrate for base modification via hydrolytic deamination of adenosines to yield inosines. Five adenosines, located within a sequence encoding the puta-tive second intracellular domain can be converted to inosines. This editing can alter the coding potential of the triplet codons and allows for the generation of multiple different receptor isoforms. The edited receptor isoforms were shown to have reduced ability to interact with G-proteins in the absence of agonist stimulation [Werry, TD, Loiacono R, Sexton P A, Christopoulos A (2008). RNA editing of the serotonin 5-HT_{2C} receptor and its effects on cell signaling, pharmacology and brain function. Pharmac. Therap. 119:7-23].

Edited 5-HT_{2C} isoforms with reduced function are significantly expressed in the brains of depressed suicide victims [Schmauss C (2003) Serotonin 2C receptors: suicide, serotonin, and runaway RNA editing. Neuroscientist 9:237-242. Iwamoto K, Kato T (2003). RNA editing of serotonin 2C receptor in human postmortem brains of major mental disorders. Neurosci. Lett. 346:169-172] and in the learned helplessness rats (a well established animal model of depression) [Iwamotoa K, Nakatanib N, Bundoa M, Yoshikawab T, Katoa T (2005). Altered RNA editing of serotonin 2C receptor in a rat model of depression. Neurosci. Res. 53: 69-76] suggesting a link between 5-HT_{2C} function and depression. There are also implications of edited 5-HT_{2C} isoforms and spatial memory [Du Y, Stasko M, Costa A C, Davissone M T, Gardiner K J (2007). Editing of the serotonin 2C receptor pre-mRNA Effects of the Morris Water Maze. Gene 391:186-197]. In addition, fully edited isoforms of the human 5-HT_{2C} receptor display a striking reduction in sensitivity to lysergic acid diethylamide (LSD) and to atypical antipsychotic drugs clozapine and loxapine, suggesting a possible role of the receptor in the etiolo-gy and pharmacology of schizophrenia [Niswender C M, Herrick-Davis K, Dilley G E, Meltzer H Y, Overholser J C, Stockmeier C A, Emeson R B, Sanders-Bush E (2001). RNA Editing of the Human Serotonin 5-HT_{2C} Receptor: Alterations in Suicide and Implications for Serotonergic Pharmacotherapy. Neuropsychopharm. 24:478-491].

Recently, the availability of potent and selective 5-HT_{2C} receptor agonists made it possible to directly investigate the effects of 5-HT_{2C} agonists and their therapeutic potential. Thus recent studies demonstrated that selective 5-HT_{2C} agonists resulted in decreased food intake and body weight gain in normal and obese rats [Smith B M, et al. (2008). Discovery and structure-activity relationship of (1R)-8-chloro-2,3,4,5-tetrahydro-1-methyl-1H-3-benzazepine (Lorcaserin), a selective serotonin 5-HT_{2C} receptor agonist for the treatment of obesity. J Med Chem 51:305-313. Thomsen W J, Grottick A J, Menzaghi F, Reyes-Saldana H, Espitia S, Yuskin D, Whelan K, Martin M, Morgan M, Chen W, Al-Shama H, Smith B, Chalmers D, Behan D (2008) Lorcaserin, A Novel Selective Human 5-HT_{2C} Agonist: In Vitro and In Vivo Pharmacological Characterization. J Pharmacol Exp Ther. 325:577-587. Rosenzweig-Lipson S, Zhang J, Mazandarani H, Harrison B L, Sabb A, Sabalski J, Stack G, Welmaker G, Barrett J E, Dunlop J (2006) Antiobesity-like effects of the 5-HT_{2C} receptor agonist WAY-161503. Brain Res. 1073-1074:240-251. Dunlop J, Sabb A L, Mazandarani H, Zhang J, Kal-gaonker S, Shukhina E, Sukoff S, Vogel R L, Stack G, Schechter L, Harrison B L, Rosenzweig-Lipson S (2005). WAY-163909 [97bR, 10aR)-1,2,3,4,8,9,10,10a-octahydro-7bH-cyclopenta-[b][1,4]diazepino[6,7,1hi]indole], a novel 5-hydroxytryptamine 2C receptor-selective agonist with anorectic activity. J Pharmacol Exp Ther. 313:862-869.].

Furthermore, selective 5-HT_{2C} receptor agonists produce antidepressant effects in animal models of depression comparable to those of SSRIs but with a much faster onset of action and a therapeutic window that avoids antidepressant-induced sexual dysfunction. These agonists were also effective in animal models of compulsive behavior such as scheduled induced polydipsia and they also exhibited decreased hyperactivity and aggression in rodents [Rosenzweig-Lipson S, Sabb A, Stack G, Mitchell P, Lucki I, Malberg J E, Grauer S, Brennan J, Cryan J F, SukoffRizzo S J, Dunlop J, Barrett J E, Marquis K L (2007) Antidepressant-like effects of the novel, selective, 5-HT_{2C} receptor agonist WAY-163909 in rodents. Psychopharmacology (Berlin) 192:159-170. Rosenzweig-Lipson S, Dunlop J, Marquis K L (2007) 5-HT_{2C} receptor agonists as an innovative approach for psychiatric disorders. Drug news Perspect, 20: 565-571. Cryan, J F, Lucki I (2000). Antidepressant-like behavioral effects mediated by 5-Hydroxytryptamine 2C receptors. J. Pharm. Exp. Ther. 295:1120-1126.].

Acute or chronic administration of 5-HT_{2C} agonists decreases the firing rate of ventral tegmental area dopamine neurons but not that of substantia nigra. In addition 5-HT_{2C} agonists reduce dopamine levels in the nucleus accumbens but not in the striatum (the region of the brain mostly associated with extrapyramidal side effects) [Di Matteo, V., Di Giovanni, G., Di Mascio, M., & Esposito, E. (1999). SB 242084, a selective serotonin 2C receptor antagonist, increases dopaminergic transmission in the mesolimbic system. Neuropharmacology 38, 1195-1205. Di Giovanni, G., Di Matteo, V., Di Mascio, M., & Esposito, E. (2000). Preferential modulation of mesolimbic vs. nigrostriatal dopaminergic function by serotonin2C/2B receptor agonists: a combined in vivo electrophysiological and microdialysis study. Synapse 35, 53-61. Marquis K L, Sabb A L, Logue S F, Brennan J A, Piesla M J, Comery T A, Grauer S M, Ashby C R, Jr., Nguyen H Q, Dawson L A, Barrett J E, Stack G, Meltzer H Y, Harrison B L, Rosenzweig-Lipson S (2007) WAY-163909 [(7bR,10aR)-1,2,3,4,8,9,10,10a-octahydro-7bH-cyclopenta-[b][1,4]diazepino[6,7,1hi]indole]: A novel 5-hydroxytryptamine 2C receptor-selective agonist with preclinical antipsychotic-like activity. J Pharmacol Exp Ther 320:486-496.]. Therefore it is expected that 5-HT_{2C} receptor agonists will selectively decrease mesolimibic dopamine levels without affecting the nigrostriatal pathway thus avoiding the EPS side effects of typical antipsychotics. Several 5-HT_{2C} receptor agonists have shown antipsychotic activity in animal models of schizophrenia without EPS based on the lack of effect in catalepsy [Marquis K L, Sabb A L, Logue S F, Brennan J A, Piesla M J, Comery T A, Grauer S M, Ashby C R, Jr., Nguyen H Q, Dawson L A, Barrett J E, Stack G, Meltzer H Y, Harrison B L, Rosenzweig-Lipson S (2007) WAY-163909 [(7bR,1 0aR)-1,2,3,4,8,9,10,10a-octahydro-7bH-cyclopenta-[b] [1,4]diazepino[6,7,1hi]indole]: A novel 5-hydroxytryptamine 2C receptor-selective agonist with pre-clinical antipsychotic-like activity. J Pharmacol Exp Ther 320:486-496. Siuciak J A, Chapin D S, McCarthy S A, Guanowsky V, Brown J, Chiang P, Marala R, Patterson T, Seymour P A, Swick A, Iredale P A (2007) CP-809,101, a selective 5-HT_{2C} agonist, shows activity in animal models of antipsychotic activity. Neuropharmacology 52:279-290]. The antipsychotic activity of 5-HT_{2C} receptor agonists without EPS coupled with their beneficial effects in mood disorders and cognition and their antiobesity like effects render 5-HT_{2C} receptor agonists as unique agents to treat schizophrenia [Rosenzweig-Lipson S, Dunlop J, Marquis K L (2007) 5-HT_{2C} receptor agonists as an innovative approach for psychiatric disorders. Drug news Perspect, 20: 565-571. Dunlop J, Marquis K L, Lim H K, Leung L, Kao J, Cheesman C, Rosenzweig-Lipson S (2006). Pharmacological profile of the 5-HT_{2C} receptor agonist WAY-163909; therapeutic potential in multiple indications. CNS Dug Rev. 12:167-177.].

In addition 5-HT_{2C} modulation has been implicated in epilepsy [Isaac M (2005). Serotonergic 5-HT_{2C} receptors as a potential therapeutic target for the antiepileptic drugs. Curr. Topics Med. Chem. 5:59:67], psoriasis [Thorslund K, Nordlind K (2007). Serotonergic drugs—a possible role in the treatment of psoriasis? Drug News Perspect 20:521-525], Parkinson's disease and related motor disorders [Esposito E, Di Matteo V, Pierucci M, Benigno A, Di Giavanni, G (2007). Role of central 5-HT_{2C} receptor in the control of basal ganglia functions. The Basal Ganglia Pathophysiology: Recent Ad-winces 97-127], behavioral deficits [Barr A M, Lahmann-Masten V, Paulus M, Gainetdinov R P, Caron M G, Geyer M A (2004). The selective serotonin-2A receptor antagonist M100907 reverses behavioral deficits in dopamine transporter knockout mice. Neuropsychopharmacology 29:221-228], anxiety [Dekeyne A, Mannoury la Cour C, Gobert A, Brocco M, Lejuene F, Serres F, Sharp T, Daszuta A, Soumier A, Papp M, Rivet J M, Flik G, Cremers T I, Muller O, Lavielle G, Millan M J (2208). S32006, a novel 5-HT_{2C} receptor antagonists displaying broad-based antidepressant and anxiolytic properties in rodent models. Psychopharmacology 199:549-568. Nunes-de-Souza V, Nunes-de-Souza R L, Rodgers R J, Canto-de-Souza A (2008). 5-HT2 receptor activation in the midbrain periaqueductal grey (PAG) reduces anxiety-like behavior in mice. Be-hav. Brain Res. 187:72-79.], migraine [Leone M, Rigamonti A, D'Amico D, Grazzi L, Usai S, Bussone G (2001). The serotonergic system in migraine. Journal of Headache and Pain 2(Suppl. 1):S43-S46], Alzheimer's disease [Arjona A A, Pooler A M, Lee R K, Wurtman R J (2002). Effect of a 5-HT_{2C} serotonin agonist, dexnorfenfluramine, on am-yloid precursor protein metabolism in guinea pigs. Brain Res. 951:135-140], pain and spinal cord injury [Nakae A, Nakai K, Tanaka T, Hagihira S, Shibata M, Ueda K, Masimo T (2008). The role of RNA editing of the serotonin 2C receptor in a rat model of oro-facial neuropathic pain. The European Journal of Neuroscience 27:2373-23 79. Nakae A, Nakai K, Tanaka T, Takashina M, Hagihira S, Shibata M, Ueda K, Mashimo T (2008). Serotonin 2C receptor mRNA editing in neuropathic pain model. Neurosci. Res. 60:228-231. Kao T, Shumsky J S, Jacob-Vadakot S, Timothy H B, Murray M, Moxon, K A (2006). Role of the 5-HT_{2C} receptor in improving weight-supported step-ping in adult rats spinalized as neonates. Brain Res. 1112:159-168.], sexual dysfunction [Motofei I G (2008). A dual physiological character for sexual function: the role of serotonergic receptors. BJU International 101:531-534. Shimada I, Maeno K, Kondoh Y, Kaku H, Sugasawa K, Kimura Y, Hatanaka K, Naitou Y, Wanibuchi F, Sakamoto S, Tsukamoto S (2008). Synthesis and structure-activity relationships of a series of benzazepine derivatives as 5-HT_{2C} receptor agonists. Bioorg. Med. Chem. 16:3309-3320.], smoking cessation [Fletcher P J, Le A D, Higgins G A (2008). Serotonin receptors as potential targets for modulation of nicotine use and dependence. Progress Brain Res. 172:361-83], substance dependence [Bubar M J, Cunningham K A (2008). Prospects for serotonin 5-HT2R pharmacotherapy in psychostimulant abuse. Progress Brain Res. 172:319-46], and ocular hypertension [Sharif N A, McLaughlin M A, Kelly C R (2006). AL-34662: a potent, selective, and efficacious ocular hypotensive serotonin-2 receptor agonist. J Ocul Pharmacol Ther. 23:1-13].

Further, 5HT modulation can be useful in the treatment of pain, both neuropathic and nociceptive pain, see for example U.S. Patent application publication US2007/0225277. Obata, Hideaki; Ito, Naomi; Sasaki, Masayuki; Saito, Shigeru; Goto, Fumio. Possible involvement of spinal noradrenergic mechanisms in the antiallodynic effect of intrathecally administered 5-HT2C receptor agonists in the rats with periph-eral nerve injury. European Journal of Pharmacology (2007), 567(1-2), 89-94. Serotonin2C receptor mRNA editing in neuropathic pain model. Nakae, Aya; Nakai, Kunihiro; Tanaka, Tatsuya; Takashina, Masaki; Hagihira, Satoshi; Shibata, Masahiko; Ueda, Koichi; Mashimo, Takashi. Department of Anesthesiology & Intensive Care Medicine, Graduate School of Medicine, Osaka University, Neuroscience Research (Amsterdam, Netherlands) (2008), 60(2), 228-231. Antiallodynic effects of intrathecally administered 5-HT2C receptor agonists in rats with nerve injury. Obata, Hideaki; Saito, Shigeru; Sakurazawa, Shinobu; Sasaki, Masayuki; Usui, Tadashi; Goto, Fumio. Department of Anesthesiology, Gunma University Graduate School of Medicine, Maebashi, Gunma, Japan. Pain (2004), 108(1-2), 163-169. Influence of 5,7-dihydroxytryptamine (5,7-DHT) on the antinociceptive effect of serotonin (5-HT) 5-HT2C receptor agonist in male and female rats. Brus, Ryszard; Kasperska, Alicja; Oswiecimska, Joanna; Szkilnik, Ryszard. Department of Pharmacology, Silesian Medical University, Zabrze, Pol. Medical Science Monitor (1997), 3(5), 654-656.

Modulation of 5H-1T2 receptors may be beneficial in the treatment of conditions related to bladder function, in particular, urinary incontinence. [Discovery of a novel azepine series of potent and selective 5-HT2C agonists as potential treatments for urinary incontinence. Brennan, Paul E. Whitlock, Gavin A. Ho, Danny K. H.; Conlon, Kelly; McMurray, Gordon. Bioorganic & Medicinal Chemistry Letters (2009), 19(17), 4999-5003. Investigation of the role of 5-HT2 receptor subtypes in the control of the bladder and the urethra in the anesthetized female rat. Mbaki, Y.; Ramage, A. G. Department of Pharmacology, University College London, London, UK. British Journal of Pharmacology (2008), 155(3), 343-356.] In particular, compounds with agonist activity at 5-HT_{2C} have been shown to be useful in treating urinary incontinence, see for example U.S. Patent application publications US2008/0146583 and US 2007/0225274.

Further pre-clinical data suggest that 5-HT_{2C} agonists could be useful for the treatment of a number of psychiatric diseases, including schizophrenia, bipolar disorders, depression/anxiety, substance use disorders and especially disorders like neuropsychiatric symptoms in Alzheimer's disease: Aggression, psychosis/agitation represent key unmet medical needs. Clinical (Shen J H Q et al., A 6-week randomized, double-blind, placebo-controlled, comparator referenced trial of vabicaserin in acute schizophrenia. Journal of Psychiatric Research 53 (2014) 14-22; Liu J et al., Prediction of Efficacy of Vabicaserin, a 5-HT_{2C} Agonist, for the Treatment of Schizophrenia Using a Quantitative Systems Pharmacology Model. CPT Pharmacometrics Syst. Pharmacol. (2014) 3, el 11;) and preclinical data (Dunlop J et al., Characterization of Vabicaserin (SCA-136), a Selective 5-Hydroxytryptamine 2C Receptor Agonist. J Pharmacol Exp Ther (2011) 337, 673-80; Siuciak J et al., CP-809,101, a selective 5-HT_{2C} agonist, shows activity in animal models of antipsychotic activity. Neuropharmacology 52 (2007) 279-290; Mosienko V et al., Exaggerated aggression and decreased anxiety in mice deficient in brain serotonin. Transl Psychiatry (2012) 2, e122; Del Guidice T et al., Stimulation of 5-HT_{2C} Receptors Improves Cognitive Deficits Induced by Human Tryptophan Hy-droxylase2 Loss of Function Mutation. Neuropsychopharmacology (2014) 39, 1125-1134; Rosenzweig-Lipson et al., Antidepressant-like effects of the novel, selective, 5-HT₂c receptor agonist WAY-163909 in rodents. Psychopharmacology (2007) 192:159-170) suggest 5-HT_{2C} receptor stimulation to result in therapeutic efficacy in aggression, psychosis agitation and moderate pro-cognitive effects (Del Guidice T et al., Stimulation of 5-HT_{2C} Receptors Improves Cognitive Deficits Induced by Human Tryptophan Hydroxylase2 Loss of Function Mutation. Neuropsychopharmacology (2014) 39, 1125-1134; Siuciak J et al., CP-809,101, a selective 5-HT_{2C} agonist, shows activity in animal models of antipsychotic activity. Neuropharmacology 52 (2007) 279-290).

In the use and the method of the invention, an effective quantity of one or more compounds, as a rule formulated in accordance with pharmaceutical and veterinary practice, is administered to the individual to be treated, preferably a mammal, in particular a human being, productive animal or domestic animal. Whether such a treatment is indicated, and in which form it is to take place, depends on the individual case and is subject to medical assessment (diagnosis) which takes into consideration signs, symptoms and/or malfunctions which are present, the risks of developing particular signs, symptoms and/or malfunctions, and other factors.

Actual dosage levels of active ingredients in the pharmaceutical compositions of the present invention can be varied so as to obtain an amount of the active compound(s) that is effective to achieve the desired therapeutic response for a particular subject (e.g., a mammal, preferably, a human (patient)), compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

Compounds of the present invention can also be administered to a subject as a pharmaceutical composition comprising the compounds of interest in combination with at least one pharmaceutically acceptable carriers. The phrase “therapeutically effective amount” of the compound of the present invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a subject (namely, a mammal, such as a human) ranges from about 0.01 mg/kg body weight to about 100 mg/kg body weight. More preferable doses can be in the range of from about 0.01 mg/kg body weight to about 30 mg/kg body weight. If desired, the effective daily dose can be divided into multiple doses for purposes of administration. Consequently, single dose compositions may contain such amounts or submultiples thereof to make up the daily dose.

In one aspect, the present invention provides pharmaceutical compositions. The pharmaceutical compositions of the present invention comprise the compounds of the present invention or an N-oxide, a tautomeric form, a stereoisomer or a pharmaceutically acceptable salt or solvate thereof. The pharmaceutical compositions of the present invention comprise compounds of the present invention that can be formulated together with at least one non-toxic pharmaceutically acceptable carrier.

In yet another embodiment, the present invention provides a pharmaceutical composition comprising compounds of the present invention or an N-oxide, a tautomeric form, a stereoisomer or a pharmaceutically acceptable salt thereof, and one or more pharmaceutically acceptable carriers, alone or in combination with one or more compounds that are not the compounds of the present invention. Examples of one or more compounds that can be combined with the compounds of the present invention in pharmaceutical compositions, include, but are not limited to, one or more cognitive enhancing drugs.

The pharmaceutical compositions of this present invention can be administered to a subject (e.g., a mammal, such as a human) orally, rectally, parenterally, intracister-nally, intravaginally, intraperitoneally, topically (as by powders, ointments or drops), bucally or as an oral or nasal spray. The term “parenterally” as used herein, refers to modes of administration which include intravenous, intramuscular, intraperitoneal, in-trasternal, subcutaneous and intraarticular injection and infusion.

The term “pharmaceutically acceptable carrier” as used herein, means a non-toxic, inert solid, semi-solid or liquid filler, diluent, encapsulating material or formula-tion auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of the present invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microor-ganisms can be ensured by the inclusion of various antibacterial and antifungal agents, for example, paraben, chlorobutanol, phenol sorbic acid and the like. It may also be desirable to include isotonic agents such as sugars, sodium chloride and the like. Pro-longed absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tet-rahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of the present invention include powders, sprays, ointments and inhalants. The active compound may be mixed under sterile conditions with a pharmaceutically acceptable carrier and any needed preservatives, buffers or propellants which may be required. Ophthalmic formulations, eye ointments, powders and solutions are also contemplated as being within the scope of this invention.

The compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq.). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hem-isulfate, heptanoate, hexanoate, fumarate, hydrochloride, hydrobromide, hydroiodide, 2-hydroxyethansulfonate (isothionate), lactate, malate, maleate, methanesulfonate, nico-tinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetrame-thylammonium, tetraethylammonium, methylammonium, dimethylammonium, trime-thylammonium, triethylammonium, diethylammonium, ethylammonium and the like. Other representative organic amines useful for the formation of base addition salts include ethylenediamine, ethanolamine, diethanolamine, piperidine, piperazine and the like.

The compounds of the present invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The following examples serve to explain the invention without limiting it.

### EXAMPLES

### Abbreviations

h, hr, hrs hour(s)
min minutes
RT room temperature (25° C.)
ACN acetonitrile
DCE dichloroethane
DCM dichloromethane
DMF dimethylformamide
DMSO dimethylsulfoxide
EtOAc ethyl acetate
EtOH ethanol
PE petrol ether
THF tetrahydrofuran
Boc, Boc, BOC tert-butoxycarbonyl
DIAD diisopropyl azodicarboxylate
DIPEA diisopropylethyl amine
HATU O-(7-azabenzotriazol-1-yl)-N,N,N′,N′-tetramethyluronium hexafluorophosphate
HBTU (O-benzotriazol-1-yl)-N,N,N′,N′-tetramethyluronium hexafluorophosphate
PS—PPh3 resin-bound triphenylphosphine [resin=crosslinked poly(styrene-codivinylbenzene)]
TFA trifluoroacetic acid

The compounds were either characterized via proton-NMR in d₆-dimethylsulfoxide, d-chloroform or d₄-methanol on a 400 MHz, 500 MHz or 600 MHz NMR instrument (Bruker AVANCE), or by ¹³C-NMR at 125 MHz, or by ¹⁹F-NMR at 470 MHz, or by mass spectrometry, generally recorded via HPLC-MS in a fast gradient on C18-material (electrospray-ionisation (ESI) mode).

The magnetic nuclear resonance spectral properties (NMR) refer to the chemical shifts (δ) expressed in parts per million (ppm). The relative area of the shifts in the ¹H-NMR spectrum corresponds to the number of hydrogen atoms for a particular functional type in the molecule. The nature of the shift, as regards multiplicity, is indicated as singlet (s), broad singlet (br s), doublet (d), broad doublet (br d), triplet (t), broad triplet (br t), quartet (q), quintet (quint.), multiplet (m), doublet of doublets (dd), doublet of doublets of doublets (ddd), triplet of doublets (td), triplet of triplets (tt), doublet of triplets of doublets (dtd), doublet of triplets of triplets (dtt), doublet of triplets of quartets (dtq), quartet of doublets (qd), quartet of doublets of doublets (qdd) etc.

### I. Synthetic Examples

### Example 1

### (2S)—N-[[5-Fluoro-4-[(3-fluorophenyl)methoxy]-2-methoxy-phenyl]methyl]-pyrrolidine-2-carboxamide and its Fumaric Acid Addition Salt

Potassium 2-methylpropan-2-olate (7.86 g, 70.0 mmol) was suspended in THF (80 mL) and the mixture was cooled to 0° C. Phenylmethanol (13.77 g, 127 mmol) was added and the solution was stirred at 0° C. for 30 min followed by dropwise addition to 2,4,5-trifluorobenzonitrile (10 g, 63.7 mmol) in THF (80 mL) at −78° C. under nitrogen. The solution was stirred at −78° C. for 3 h, warmed to room temperature over 1.5 h and stirred at RT for 20 h. The solution was diluted with EtOAc (500 mL) and washed with water (2*70 mL). The water phases were re-extracted with DCM (100 mL*2) and the combined organic phases were concentrated to give crude product, which was recrystallized from EtOH and resulted in 4-(benzyloxy)-2,5-difluorobenzonitrile (10.5 g, 42.8 mmol, 67.3% yield).

Potassium 2-methylpropan-2-olate (5.86 g, 52.2 mmol) was suspended in THF (50 mL) and the mixture was cooled to 0° C. Methanol (1.672 g, 52.2 mmol) was added and the solution was stirred at 0° C. for 30 min followed by dropwise addition to 4-(benzyloxy)-2,5-difluorobenzonitrile (8 g, 32.6 mmol) in THF (50 mL) at −50° C. under nitrogen. The solution was stirred at −50° C. for 1 h, warmed to room temperature over 0.5 h and stirred at RT for 20 h. The solution was diluted with EtOAc (500 mL) and washed with water (2*70 mL). The water phases were re-extracted with DCM (200 mL*2) and the combined organic phases were concentrated to give crude product, which was recrystallized from EtOH resulting in 4-(benzyloxy)-5-fluoro-2-methoxybenzonitrile (6.7 g, 26.0 mmol, 80% yield) as white solid.

To a mixture of 4-(benzyloxy)-5-fluoro-2-methoxybenzonitrile (3 g, 11.66 mmol) in methanol (15 mL), 1.5 mL conc. HCl and Pd/C (150 mg) were added, the resulting mixture was evacuated and filled with hydrogen (0.118 g, 58.3 mmol), and the resulting mixture was stirred at 40° C. under hydrogen (0.118 g, 58.3 mmol) for 16 h. Data LCMS showed the consumption of the starting material. Then the mixture was filtered, the fil-trate was concentrated under reduced pressure to give a residue. The residue was dissolved in 25 mL EtOAc, filtered, the solid was collected, 4-(aminomethyl)-2-fluoro-5-methoxyphenol hydrochloride (1.9 g, 9.24 mmol, 79% yield) was obtained as light yellow solid.

To a mixture of (S)-1-(tert-butoxycarbonyl)pyrrolidine-2-carboxylic acid (3 g, 13.94 mmol) in DMF (16 mL), DIPEA (6.29 g, 48.8 mmol) and HATU (6.36 g, 16.73 mmol) were added, and the mixture was stirred at room temperature for 20 min. After that, 4-(aminomethyl)-2-fluoro-5-methoxyphenol hydrochloride (3.15 g, 15.33 mmol) was added, and the resulting mixture was stirred at room temperature overnight. Data LCMS showed ms of the desired product. The reaction mixture was diluted in 160 mL DCM, then the mixture was washed with water (60 mL*4) for four times, the organic layer was dried over Na₂SO₄, filtered, concentrated and the residue was purified through column (silica gel; UV 254) eluting with 0-50% EtOAc/PE to give a residue, which was washed with mixed solvents (EtOAc/PE=1:2), white solid was precipitated, filtered, the solid was collected. (S)-tert-butyl 2-((5-fluoro-4-hydroxy-2-methoxybenzyl)-carbamoyl)pyrrolidine-1-carboxylate (3.3 g, 8.96 mmol, 64.3% yield) was obtained as white solid.

(S)-tert-butyl 2-((5-fluoro-4-hydroxy-2-methoxybenzyl)carbamoyl)pyrrolidine-1-carboxylate (50 mg, 0.136 mmol) was dissolved in DMF (3 mL), cesium carbonate (88 mg, 0.271 mmol) was added and the reaction mixture stirred for 15 min at room temperature. Subsequently 1-(bromomethyl)-3-fluorobenzene (0.017 mL, 0.136 mmol) was added and the reaction mixture stirred overnight at room temperature. Water was added, the reaction mixture extracted three times with ethyl acetate, and the organic phases were combined and dried over sodium sulfate. Evaporation of the solvent and purification of the raw material by column chromatography resulted in (S)-tert-butyl 2-((5-fluoro-4-((3-fluorobenzyl)oxy)-2-methoxybenzyl)carbamoyl)pyrrolidine-1-carboxylate as white foam (58 mg; 90% yield).

(S)-tert-butyl 2-((5-fluoro-4-((3-fluorobenzyl)oxy)-2-methoxybenzyl)carbamoyl)-pyrrolidine-1-carboxylate (58 mg, 0.122 mmol) was dissolved in DCM (4 mL) 2,2,2-trifluoroacetic acid (0.234 mL, 3.04 mmol) was added and the reaction mixture stirred overnight at room temperature. After further dilution with DCM the reaction mixture was extracted three times with 1M NaOH to remove 2,2,2-trifluoroacetic acid, the organic phase was dried over sodium sulfate and the solvent evaporated in vacuum. (S)—N-(5-fluoro-4-((3-fluorobenzyl)oxy)-2-methoxybenzyl)pyrrolidine-2-carboxamide (23 mg; 49% yield) was obtained as film in the vessel.

(S)—N-(5-fluoro-4-((3-fluorobenzyl)oxy)-2-methoxybenzyl)pyrrolidine-2-carboxamide (16.6 mg, 0.044 mmol) was dissolved in ethanol (0.7 mL), fumaric acid (5.1 mg, 0.0441 mmol) was added, and the mixture stirred at room temperature for 1 h. The solvent was evaporated, water was added to the residue and the solution lyophilized to obtain (S)—N-(5-fluoro-4-((3-fluorobenzyl)oxy)-2-methoxybenzyl)pyrrolidine-2-carboxamide fumarate as white powder (19.6 mg; 89% yield).

LC-MS (M/Z [M+H]⁺): 377.1

¹H NMR (600 MHz, methanol-d₄) δ 7.39 (td, J=7.9, 5.8 Hz, 1H), 7.28-7.24 (m, 1H), 7.19 (dt, J=9.9, 2.0 Hz, 1H), 7.08-7.00 (m, 2H), 6.77 (d, J=7.0 Hz, 1H), 6.69 (s, 2H), 5.19 (s, 2H), 4.34, 4.30 (ABq, J=14.7 Hz, 2H), 4.19 (dd, J=8.4, 7.0 Hz, 1H), 3.80 (s, 3H), 3.38 (dt, J=11.4, 7.1 Hz, 1H), 3.31-3.25 (m, 1H), 2.41-2.35 (m, 1H), 2.07-2.00 (m, 2H), 2.00-1.92 (m, 1H).

### Example 2

### (2S)—N-[[5-Fluoro-2-methoxy-4-[(2,3,5-trifluorophenyl)methoxy]phenyl]methyl]-pyrrolidine-2-carboxamide and its 2,2,2-trifluoroacetic Acid Addition Salt

A 4 mL vial was charged with a stir bar, to which was added (S)-tert-butyl 2-((5-fluoro-4-hydroxy-2-methoxybenzyl)carbamoyl)pyrrolidine-1-carboxylate (30 mg, 1 eq., 0.08 mmol; see example 1) in 0.5 ml of dimethyl acetamide followed by 1-(bromomethyl)-2,3,5-trifluorobenzene (22 mg, 1.2 eq, 0.10 mmol) in 0.5 ml of dimethyl acetamide and cesium carbonate (80 mg, 024 mmol, 3 eq.). This mixture was allowed to stir at 60° C. overnight until completion of reaction. Upon completion, the mixture was then filtered and concentrated to dryness. The residue was dissolved in 0.5 ml of 1:1 TFA:Dichloromethane and allowed to stir for 6 hours. Upon completion of the deprotection, the reaction was dried and purified by reverse phase HPLC (TFA method). Samples were purified by preparative HPLC on a Phenomenex Luna C8(2) 5 um 100 Å AXIA column (30 mm×150 mm). A gradient of ACN (A) and 0.1% trifluoroacetic acid in water (B) was used, at a flow rate of 50 mL/min (0-0.5 min 5% A, 0.5-8.5 min linear gradient 5-100% A, 8.7-10.7 min 100% A, 10.7-11.0 min linear gradient 100-5% A). to afford the desired compound (2S)—N-[[5-fluoro-2-methoxy-4-[(2,3,5-trifluorophenyl)methoxy]phenyl]methyl]pyrrolidine-2-carboxamide as 2,2,2-trifluoroacetic acid salt.

MS(APCI+) (M/Z [M+H]⁺): 413

¹H NMR (400 MHz, DMSO-d₆) δ 8.80 (t, J=5.7 Hz, 1H), 7.53 (dddd, J=10.8, 9.1, 6.2, 3.1 Hz, 1H), 7.28 (ddt, J=7.7, 4.9, 2.7 Hz, 1H), 7.06 (d, J=11.7 Hz, 1H), 6.96 (d, J=7.2 Hz, 1H), 5.34-5.25 (m, 2H), 4.24 (t, J=3.0 Hz, 2H), 4.17 (t, J=7.8 Hz, 1H), 3.31-3.16 (m, 2H), 2.35-2.25 (m, 1H), 1.96-1.78 (m, 3H).

### Example 3

### (2S)—N-[[5-Fluoro-4-[(4-fluorophenyl)methoxy]-2-methoxy-phenyl]methyl]-pyrrolidine-2-carboxamide and its 2,2,2-trifluoroacetic Acid Addition Salt

The compound was prepared according to example 2 starting from 1-(bromomethyl)-4-fluorobenzene and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[5-fluoro-4-[(4-fluorophenyl)methoxy]-2-methoxy-phenyl]methyl]pyrrolidine-2-carboxamide 2,2,2-trifluoroacetic acid salt was obtained.

MS(APCI+) (M/Z [M+H]⁺): 377

¹H NMR (400 MHz, DMSO-d₆) δ 7.52 (dd, J=8.6, 5.6 Hz, 2H), 7.28-7.19 (m, 2H), 7.03 (d, J=11.8 Hz, 1H), 6.91 (d, J=7.3 Hz, 1H), 5.19 (s, 2H), 4.22 (s, 2H), 4.16 (t, J=7.9 Hz, 1H), 3.23 (ddt, J=31.4, 11.3, 7.2 Hz, 2H), 2.31 (dt, J=13.6, 6.3 Hz, 1H), 1.95-1.76 (m, 3H).

### Example 4

### (2S)—N-[[4-[(2,3-Difluorophenyl)methoxy]-5-fluoro-2-methoxy-phenyl]methyl]-pyrrolidine-2-carboxamide and its 2,2,2-trifluoroacetic Acid Addition Salt

The compound was prepared according to example 2 starting from 1-(bromomethyl)-2,3-difluorobenzene and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[4-[(2,3-difluorophenyl)methoxy]-5-fluoro-2-methoxy-phenyl]methyl]pyrrolidine-2-carboxamide 2,2,2-trifluoroacetic acid salt was obtained.

MS(APCI+) (M/Z [M+H]⁺): 395

¹H NMR (400 MHz, DMSO-d₆) δ 8.79 (t, J=5.7 Hz, 1H), 7.52-7.34 (m, 2H), 7.27 (tdd, J=8.1, 5.0, 1.5 Hz, 1H), 7.04 (d, J=11.7 Hz, 1H), 6.96 (d, J=7.2 Hz, 1H), 5.29 (s, 2H), 4.24 (t, J=3.0 Hz, 2H), 4.17 (t, J=7.9 Hz, 1H), 3.33-3.14 (m, 2H), 2.32 (ddd, J=14.9, 12.4, 6.7 Hz, 1H), 1.98-1.90 (m, 2H), 1.90-1.77 (m, 1H).

### Example 5

### (2S)—N-[[4-[(3,5-Difluorophenyl)methoxy]-5-fluoro-2-methoxy-phenyl]methyl]-pyrrolidine-2-carboxamide and its 2,2,2-trifluoroacetic Acid Addition Salt

The compound was prepared according to example 2 starting from 1-(bromomethyl)-3,5-difluorobenzene and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[4-[(3,5-difluorophenyl)methoxy]-5-fluoro-2-methoxy-phenyl]methyl]pyrrolidine-2-carboxamide 2,2,2-trifluoroacetic acid salt was obtained.

MS(APCI+) (M/Z [M+H]⁺): 395

¹H NMR (400 MHz, DMSO-d₆) δ 7.24-7.12 (m, 3H), 7.06 (d, J=11.7 Hz, 1H), 6.90 (d, J=7.3 Hz, 1H), 5.25 (s, 2H), 4.23 (d, J=4.1 Hz, 2H), 4.17 (t, J=7.9 Hz, 1H), 3.79 (s, 3H), 3.33-3.14 (m, 2H), 2.39-2.26 (m, 1H), 1.98-1.90 (m, 2H), 1.90-1.76 (m, 1H).

### Example 6

### (2S)—N-[[5-Fluoro-4-[(2-fluorophenyl)methoxy]-2-methoxy-phenyl]methyl]-pyrrolidine-2-carboxamide and its 2,2,2-trifluoroacetic Acid Addition Salt

The compound was prepared according to example 2 starting from 1-(bromomethyl)-2-fluorobenzene and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[5-fluoro-4-[(2-fluorophenyl)methoxy]-2-methoxy-phenyl]methyl]pyrrolidine-2-carboxamide 2,2,2-trifluoroacetic acid salt was obtained.

MS(APCI+) (M/Z [M+H]⁺): 377

¹H NMR (400 MHz, DMSO-d₆) δ 7.56 (td, J=7.6, 1.8 Hz, 1H), 7.51-7.40 (m, 1H), 7.31-7.21 (m, 2H), 7.04 (d, J=11.7 Hz, 1H), 6.95 (d, J=7.2 Hz, 1H), 5.24 (s, 2H), 4.23 (s, 2H), 4.17 (t, J=7.9 Hz, 1H), 3.82 (s, 3H), 3.23 (ddt, J=31.2, 11.4, 7.1 Hz, 2H), 2.31 (ddd, J=12.7, 8.3, 6.3 Hz, 1H), 1.98-1.87 (m, 2H), 1.87-1.76 (m, 1H).

### Example 7

### (2S)—N-[[4-[(2-Chloro-3,6-difluoro-phenyl)methoxy]-5-fluoro-2-methoxy-phenyl]-methyl]pyrrolidine-2-carboxamide and its 2,2,2-trifluoroacetic Acid Addition Salt

The compound was prepared according to example 2 starting from 2-(bromomethyl)-3-chloro-1,4-difluorobenzene and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[4-[(2-chloro-3,6-difluoro-phenyl)methoxy]-5-fluoro-2-methoxy-phenyl]methyl]pyrrolidine-2-carboxamide 2,2,2-trifluoroacetic acid salt was obtained.

MS(APCI+) (M/Z [M+H]⁺): 429

¹H NMR (400 MHz, DMSO-d₆) δ 8.80 (t, J=5.7 Hz, 1H), 7.62-7.53 (m, 1H), 7.39 (td, J=9.1, 4.2 Hz, 1H), 7.06-6.98 (m, 2H), 5.31 (d, J=1.8 Hz, 2H), 4.24 (t, J=3.0 Hz, 2H), 4.17 (t, J=7.8 Hz, 1H), 3.31-3.15 (m, 2H), 2.37-2.25 (m, 1H), 1.97-1.76 (m, 3H).

### Example 8

### (2S)—N-[[5-fluoro-4-[(2-fluoro-4-pyridyl)methoxy]-2-methoxy-phenyl]methyl]-pyrrolidine-2-carboxamide and its 2,2,2-trifluoroacetic Acid Addition Salt

The compound was prepared according to example 2 starting from 4-(bromomethyl)-2-fluoropyridine and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[5-fluoro-4-[(2-fluoro-4-pyridyl)methoxy]-2-methoxy-phenyl]methyl]pyrrolidine-2-carboxamide 2,2,2-trifluoroacetic acid salt was obtained.

MS(APCI+) (M/Z [M+H]⁺): 378

### Example 9

### (2S)—N-[[5-Fluoro-4-[(5-fluoro-3-pyridyl)methoxy]-2-methoxy-phenyl]methyl]-pyrrolidine-2-carboxamide and its 2,2,2-trifluoroacetic Acid Addition Salt

The compound was prepared according to example 2 starting from 3-(bromomethyl)-5-fluoropyridine and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[5-fluoro-4-[(5-fluoro-3-pyridyl)methoxy]-2-methoxy-phenyl]methyl]pyrrolidine-2-carboxamide 2,2,2-trifluoroacetic acid salt was obtained.

MS(APCI+) (M/Z [M+H]⁺): 378

¹H NMR (400 MHz, DMSO-d₆) δ 8.57 (d, J=2.8 Hz, 1H), 8.55 (d, J=1.8 Hz, 1H), 7.87-7.75 (m, 1H), 7.06 (d, J=11.8 Hz, 1H), 6.95 (d, J=7.3 Hz, 1H), 5.31 (s, 2H), 4.24 (d, J=4.0 Hz, 2H), 4.17 (t, J=7.8 Hz, 1H), 3.81 (s, 3H), 3.23 (ddt, J=30.6, 11.5, 7.1 Hz, 2H), 2.32 (ddd, J=15.0, 12.4, 6.5 Hz, 1H), 1.88 (ddt, J=31.4, 13.3, 7.2 Hz, 3H).

### Example 10

### (2S)—N-[[5-fluoro-2-methoxy-4-[[6-(trifluoromethyl)-3-pyridyl]methoxy]phenyl]-methyl]pyrrolidine-2-carboxamide and its 2,2,2-trifluoroacetic Acid Addition Salt

The compound was prepared according to example 2 starting from 5-(bromomethyl)-2-(trifluoromethyl)pyridine and (S)-tert-butyl 2-((5-fluoro-4-hydroxy-2-methoxybenzyl)carbamoyl)pyrrolidine-1-carboxylate. After BOC deprotection and purification by reverse phase HPLC the desired product (2S)—N-[[5-fluoro-2-methoxy-4-[[6-(trifluoromethyl)-3-pyridyl]methoxy]phenyl]methyl]pyrrolidine-2-carboxamide 2,2,2-trifluoroacetic acid salt was obtained.

MS(APCI+) (M/Z [M+H]⁺): 428

¹H NMR (400 MHz, DMSO-d₆) δ 8.85 (s, 1H), 8.17 (d, J=7.7 Hz, 1H), 8.01-7.91 (m, 1H), 7.06 (d, J=11.7 Hz, 1H), 6.96 (d, J=7.2 Hz, 1H), 5.39 (s, 2H), 4.23 (s, 2H), 4.17 (t, J=7.9 Hz, 1H), 3.29-3.15 (m, 2H), 2.37-2.25 (m, 1H), 1.97-1.77 (m, 3H).

### Example 11

### (2S)—N-[[5-Fluoro-4-[2-(4-fluorophenyl)ethoxy]-2-methoxy-phenyl]methyl]-pyrrolidine-2-carboxamide and its 2,2,2-trifluoroacetic Acid Addition Salt

In a 4 ml vial was added 200 mg of PS—PPh3 followed by (S)-tert-butyl 2-((5-fluoro-4-hydroxy-2-methoxybenzyl)carbamoyl)pyrrolidine-1-carboxylate (35 mg. 0.1 mmol) dissolved in 2 ml of THF, and 2-(4-fluorophenyl)ethanol (28 mg, 0.2 mmol) dissolved in 0.3 mL THF. The mixture was allowed to react for 10 minutes. 50 L of DIAD was then added and vial was sealed and shaken for 72 h. The reaction was filtered, and concentrated to dryness. Then 1.0 mL of HCl/dioxane was added and reaction was shaken for 1 hour. After that the reaction was concentrated to dryness again and was re-dissolved in 1800 μL of a 1:1 v/v solution of DMSO/MeOH, checked by LC/MS and purified by reverse phase HPLC (TFA method) to provide the desired product (2S)—N-[[5-fluoro-4-[2-(4-fluorophenyl)ethoxy]-2-methoxy-phenyl]methyl]pyrrolidine-2-carboxamide which was obtained as 2,2,2-trifluoroacetic acid salt (66% yield).

MS(APCI+) (M/Z [M+H]⁺): 391

¹H NMR (400 MHz, DMSO-d₆/D₂O, Temp=27° C.) 6 8.76 (t, J=5.8 Hz, 1H), 7.40-7.30 (m, 2H), 7.19-7.07 (m, 2H), 6.99 (d, J=11.8 Hz, 1H), 6.76 (d, J=7.2 Hz, 1H), 4.27 (t, J=6.6 Hz, 2H), 4.24-4.18 (m, 2H), 4.15 (t, J=7.8 Hz, 1H), 3.77 (s, 3H), 3.30-3.13 (m, 2H), 3.03 (t, J=6.6 Hz, 2H), 2.37-2.24 (m, 1H), 1.97-1.84 (m, 2H), 1.84-1.72 (m, 1H)

### Example 12

### (2S)—N-[[5-Fluoro-4-[2-(3-fluorophenyl)ethoxy]-2-methoxy-phenyl]methyl]-pyrrolidine-2-carboxamide and its 2,2,2-trifluoroacetic Acid Addition Salt

The compound was prepared according to example 11 by Mitsunobu reaction starting from 2-(3-fluorophenyl)ethanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-4-[2-(3-fluorophenyl)ethoxy]-2-methoxy-phenyl]methyl]pyrrolidine-2-carboxamide was obtained as 2,2,2-trifluoroacetic acid salt (58% yield).

MS(APCI+) (M/Z [M+H]⁺): 391

¹H NMR (400 MHz, DMSO-d₆/D₂O, Temp=27° C.) δ 8.76 (t, J=5.7 Hz, 1H), 7.46-7.28 (m, 1H), 7.24-7.12 (m, 2H), 7.07-7.02 (m, 1H), 7.00 (d, J=11.8 Hz, 1H), 6.77 (d, J=7.3 Hz, 1H), 4.30 (t, J=6.6 Hz, 2H), 4.25-4.18 (m, 2H), 4.15 (t, J=7.9 Hz, 1H), 3.78 (s, 3H), 3.35-3.11 (m, 2H), 3.06 (t, J=6.5 Hz, 2H), 2.38-2.19 (m, 1H), 1.97-1.84 (m, 2H), 1.86-1.73 (m, 1H).

### Example 13

### (2S)—N-[[5-Fluoro-4-[2-(2-fluorophenyl)ethoxy]-2-methoxy-phenyl]methyl]-pyrrolidine-2-carboxamide and its 2,2,2-trifluoroacetic Acid Addition Salt

The compound was prepared according to example 11 by Mitsunobu reaction starting from 2-(2-fluorophenyl)ethanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-4-[2-(2-fluorophenyl)ethoxy]-2-methoxy-phenyl]-methyl]pyrrolidine-2-carboxamide was obtained as 2,2,2-trifluoroacetic acid salt (42% yield).

MS(APCI+) (M/Z [M+H]⁺): 391

¹H NMR (400 MHz, DMSO-d₆/D₂O, Temp=27° C.) δ 8.77 (t, J=5.8 Hz, 1H), 7.50-7.38 (m, 1H), 7.38-7.25 (m, 1H), 7.24-7.11 (m, 2H), 7.01 (d, J=11.7 Hz, 1H), 6.79 (d, J=7.3 Hz, 1H), 4.31 (t, J=6.7 Hz, 2H), 4.25-4.20 (m, 2H), 4.16 (t, J=7.8 Hz, 1H), 3.79 (s, 3H), 3.33-3.14 (m, 2H), 3.09 (t, J=6.6 Hz, 2H), 2.43-2.23 (m, 1H), 2.00-1.87 (m, 2H), 1.87-1.73 (m, 1H)

### Example 14

### (2S)—N-[[5-Fluoro-2-methoxy-4-[2-[4-(trifluoromethyl)phenyl]ethoxy]phenyl]-methyl]pyrrolidine-2-carboxamide and its 2,2,2-trifluoroacetic Acid Addition Salt

The compound was prepared according to example 11 by Mitsunobu reaction starting from 2-(4-(trifluoromethyl)phenyl)ethanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-[2-[4-(trifluoromethyl)phenyl]-ethoxy]phenyl]methyl]pyrrolidine-2-carboxamide which was obtained as 2,2,2-trifluoroacetic acid salt (40% yield).

MS(APCI+) (M/Z [M+H]⁺): 441

¹H NMR (400 MHz, DMSO-d₆/D₂O, Temp=27° C.) 6 8.77 (t, J=5.7 Hz, 1H), 7.73-7.58 (m, 2H), 7.58-7.49 (m, 2H), 7.01 (d, J=11.8 Hz, 1H), 6.78 (d, J=7.3 Hz, 1H), 4.35 (t, J=6.5 Hz, 2H), 4.28-4.20 (m, 2H), 4.16 (t, J=7.8 Hz, 1H), 3.79 (s, 3H), 3.31-3.18 (m, 2H), 3.18-3.10 (m, 2H), 2.37-2.22 (m, 1H), 2.00-1.85 (m, 2H), 1.85-1.75 (m, 1H)

### Example 15

### (2S)—N-[[5-fluoro-2-methoxy-4-[[5-(trifluoromethyl)-2-furyl]methoxy]phenyl]-methyl]pyrrolidine-2-carboxamide and its 2,2,2-trifluoroacetic Acid Addition Salt

The compound was prepared according to example 2 starting from 2-(bromo-methyl)-5-(trifluoromethyl)furane and (S)-tert-butyl 2-((5-fluoro-4-hydroxy-2-methoxy-benzyl)carbamoyl)pyrrolidine-1-carboxylate. After BOC deprotection and purification by reverse phase HPLC the desired product (2S)—N-[[5-fluoro-2-methoxy-4-[[5-(trifluoromethyl)-2-furyl]methoxy]phenyl]methyl]pyrrolidine-2-carboxamide 2,2,2-trifluoroacetic acid salt was obtained.

MS(APCI+) (M/Z [M+H]⁺): 417

¹H NMR (400 MHz, DMSO-d₆) δ 8.79 (t, J=5.8 Hz, 1H), 7.22 (dt, J=3.6, 1.3 Hz, 1H), 7.04 (d, J=11.7 Hz, 1H), 6.96 (d, J=7.2 Hz, 1H), 6.83 (d, J=3.5 Hz, 1H), 5.27 (s, 2H), 4.24 (t, J=2.9 Hz, 2H), 4.17 (t, J=7.9 Hz, 1H), 3.82 (s, 3H), 3.23 (ddt, J=30.9, 11.4, 7.1 Hz, 2H), 2.32 (ddd, J=14.8, 12.4, 6.4 Hz, 1H), 1.98-1.87 (m, 2H), 1.87-1.76 (m, 1H).

### Example 16

### (S)—N-(5-Fluoro-4-((cis-4-fluorocyclohexyl)oxy)-2-methoxybenzyl)pyrrolidine-2-carboxamide and its Fumaric Acid Addition Salt

Ketone Reduction:

5.0 g 4-fluorocyclohexanone (43.1 mmol) was dissolved in methanol (290 mL) and cooled to 0° C. with an ice-water bath. Then, 2.44 g sodium borohydride (64.6 mmol, 1.5 eq.) was added in portions while keeping the temperature. The reaction mixture was allowed to reach room temperature and stirred overnight. TLC (ethyl acetate/heptane 1:9) indicated full conversion of the starting ketone. The methanolic solution was concentrated in vacuum, diluted with ethyl acetate and carefully quenched with water, and further neutralized with aqueous HCl. The organic phase was separated and the aqueous phase extracted with ethyl acetate (2×) again. Combined extracts were dried over Na₂SO₄ and the solvent was removed in vacuum. The alcohol remained as a brownish oil which slowly started crystallizing upon standing. Yield: 5.03 g (42.65 mmol, 99%) as a 2:1 mixture of cis/trans-isomers (A:B, A=cis, B=trans) as indicated by proton NMR.

¹H NMR (500 MHz, Chloroform-d): δ 1.38-1.80 (m, CH₂), 1.90-2.10 (m, CH₂), 3.67-3.76 (m, 1H, CHOH, isomer A), 3.83 (tt, J=7.8, 3.5 Hz, 1H, CHOH, isomer B), 4.63 (dtt, J=48.5, 8.2, 3.5 Hz, 1H, CHF, isomer B), 4.69 (dtt, J=48.8, 5.5, 2.7 Hz, 1H, CHF, isomer A).

¹⁹F NMR (471 MHz, Chloroform-d): δ−181.77-−179.97 (m, isomer B), −179.86-−178.49 (m, isomer A).

LC-MS: No ionization observed.

Benzoylation:

To a solution of 5.0 g 4-fluorocyclohexanone (42.3 mmol, cis/trans-mixture) in dichloromethane (250 mL) was added 30 mL triethylamine (215 mmol, 5.1 eq.) and slowly 10 mL benzoyl chloride (86 mmol, 2.0 eq.) at room temperature. Stirring was continued overnight in order to reach full conversion. Then, the reaction mixture was washed with aqueous ammonium chloride (2×) and with water. The organic layer was dried over MgSO₄ and the solvent evaporated. The crude benzoates were absorbed on Celite and purified by flash chromatography on silica (ethyl acetate/heptane). Yield: 2.32 g (10.44 mmol, 25%) of trans isomer (1r,4r)-4-fluorocyclohexyl benzoate as colorless oil and 5.50 g (24.75 mmol, 59%) of cis isomer (1s,4s)-4-fluorocyclohexyl benzoate as white solid.

Cis-Isomer:

¹H NMR (600 MHz, Chloroform-d): δ 1.73-1.88 (m, 4H), 1.94-2.12 (m, 4H), 4.74 (dtt, J=48.5, 6.4, 3.0 Hz, 1H, CHF), 5.08 (ddt, J=11.7, 8.1, 3.4 Hz, 1H, CHOBz), 7.43-7.49 (m, 2H), 7.54-7.60 (m, 1H), 8.03-8.08 (m, 2H).

LC-MS: No ionization observed.

Trans Isomer:

¹H NMR (600 MHz, Chloroform-d): δ 1.70-1.90 (m, 4H), 1.96-2.13 (m, 4H), 4.80 (dtt, J=51.3, 6.6, 2.9 Hz, 1H, CHF), 5.18 (tt, J=6.8, 3.1 Hz, 1H, CHOBz), 7.40-7.49 (m, 2H), 7.54-7.60 (m, 1H), 8.02-8.05 (m, 2H).

LC-MS: No ionization observed.

Hydrolysis:

Trans Isomer:

2.32 g trans-4-fluorocyclohexyl benzoate (10.44 mmol) was dissolved in a mixture of THF (21 mL), methanol (31 mL) as well as water (52 mL) and 350 mg lithium hydroxide (14.6 mmol, 1.4 eq.) was added. The reaction mixture was stirred at room temperature overnight. Volatiles were removed in vacuum and the aqueous concentrate was extracted with ethyl acetate (2×). Combined extracts were washed with water (2×), brine (1×), dried over MgSO₄ and carefully evaporated to dryness. The trans-4-fluorocyclohexanol remained as colorless oil. Yield: 1.09 g (9.23 mmol, 88%).

¹H NMR (600 MHz, Chloroform-d): δ=1.43 (dtd, J=13.7, 9.5, 9.0, 3.9 Hz, 2H), 1.59-1.69 (m, 2H), 1.92-2.10 (m, 5H), 3.84 (tt, J=7.9, 3.6 Hz, 1H, CHOH), 4.64 (dtt, J=48.7, 8.2, 3.7 Hz, 1H, CHF).

Cis Isomer:

Cis-4-fluorocyclohexanol was prepared in a similar fashion (scale: 24.75 mmol).

Yield: 2.54 g (21.5 mmol, 87%).

¹H NMR (600 MHz, Chloroform-d): δ=1.53-1.80 (m, 6H), 1.99-2.10 (m, 2H), 3.72 (dddd, J=12.9, 7.0, 5.1, 3.3 Hz, 1H, CHOH), 4.70 (dtt, J=48.5, 5.4, 2.7 Hz, 1H, CHF).

Nucleophilic Reaction:

170 mg Sodium hydride (60%, 4.26 mmol, 2.4 eq.) were dispersed in DMF (5 mL) under a nitrogen atmosphere. 251 mg cis-4-fluorocyclohexanol (2.13 mmol, 1.2 eq.) dissolved in DMF (3 mL) were added at room temperature and the mixture was stirred for 10 min. Finally, 300 mg 4,5-difluoro-2-methoxybenzonitrile (1.77 mmol) were dissolved in DMF (3 mL) and added. The reaction mixture was heated up to 60° C. and was stirred for 1 hour. It was allowed to cool down to room temperature and stirring was continued overnight. The reaction mixture was partitioned between ethyl acetate and saturated sodium bicarbonate. The organic layer was separated and the aqueous layer was extracted with ethyl acetate (2×). The combined organic phases were dried with MgSO₄ and evaporated to dryness. The residue was purified flash chromatography on silica (ethyl acetate/heptane). 5-Fluoro-4-((cis-4-fluorocyclohexyl)oxy)-2-methoxybenzonitrile was obtained as a colorless resin. Yield: 339 mg (1.27 mmol, 72%).

LC-MS (M/Z [M+H]⁺): 268.20

Nitrile Reduction:

339 mg 5-Fluoro-4-((cis-4-fluorocyclohexyl)oxy)-2-methoxybenzonitrile (1.27 mmol) were dissolved in THF (10 mL) and heated to reflux. Then, 0.3 mL of borane dimethyl sulfide complex (3.17 mmol, 2.5 q.) was added via syringe and the mixture was refluxed for 2 hours. After the reaction mixture had reached room temperature, excess hydride was carefully decomposed by addition of 2.5 M HCl in ethanol until hydrogen evolution was completed. Stirring was continued for 15 min. All volatiles were evaporated and the crude resin co-distilled with toluene (2×). The solid residue was precipitated with diethylether and dried in vacuum. The (5-fluoro-4-((cis-4-fluorocyclohexyl)oxy)-2-methoxyphenyl)methanamin hydrochloride was obtained as white crystals. Yield: 393 mg (1.27 mmol, 100%).

LC-MS (M/Z [M-NH₃]⁺): 255.25

Peptide Coupling:

275 mg L-Boc proline (1.27 mmol, 1.0 eq.) were dissolved in DMF (7.5 mL) and 228 mg carbonyldiimidazol (1.40 mmol, 1.1 eq.) were added at room temperature. The mixture was heated to 50° C. and stirred for 30 min. 393 mg (5-fluoro-4-((cis-4-fluorocyclohexyl)oxy)-2-methoxyphenyl)methanamin hydrochloride (1.27 mmol) were dissolved in pyridine (7.5 mL) and stirred for 30 min at room temperature. This pyridine solution of the amine was added to the imidazolide via syringe, the mixture was heated to 80° C. for 2 hours and stirring was continued at room temperature overnight. The reaction mixture was concentrated in vacuum and co-distilled with toluene (2×). The residue was purified by flash chromatography on silica (dichloromethane/methanol). (S)-tert-Butyl 2-((5-fluoro-4-((cis-4-fluorocyclohexyl)oxy)-2-methoxybenzyl)carbamoyl)-pyrrolidine-1-carboxylate was obtained as yellowish resin. Yield: 596 mg (1.27 mmol, 100%)

LC-MS (M/Z [M+H]⁺): 469.35

Deprotection:

596 mg (S)-tert-Butyl 2-((5-fluoro-4-((cis-4-fluorocyclohexyl)oxy)-2-methoxybenzyl)carbamoyl)pyrrolidine-1-carboxylate (1.27 mmol) were dissolved in dichloromethane (20 mL) and cooled down with an ice-water bath. Then, 2.5 mL TFA (31.8 mmol, 25 eq.) were added and the mixture was stirred at 0° C. for 1 h before it was allowed to reach room temperature and stirred overnight. The reaction mixture was partitioned between saturated sodium bicarbonate and dichloromethane. The organic phase was separated and the aqueous phase extracted with dichloromethane (2×). The combined organic phases were dried with MgSO₄ and evaporated to dryness. The product was obtained as colorless resin. The product was further purified by flash chromatography on silica (dichloromethane/methanol). (S)—N-(5-fluoro-4-(((1s,4R)-4-fluorocyclohexyl)oxy)-2-methoxybenzyl)pyrrolidine-2-carboxamide Yield: 327 mg (0.89 mmol, 70%).

LC-MS (M/Z [M+H]⁺): 369.35

Salt Formation:

103 mg Fumaric acid (0.89 mmol, 1.0 eq.) were dissolved in warm ethanol (2 mL) and added to a warm solution of 327 mg (S)—N-(5-fluoro-4-((cis-4-fluorocyclohexyl)oxy)-2-methoxybenzyl)pyrrolidine-2-carboxamide (0.89 mmol) in ethanol (10 mL). Cooling of the solution did not result in crystallization, therefore the solution was evaporated to dryness. The residue was taken up with a little methanol and diluted with dichloromethane. The solution was concentrated in vacuum, resulting in white, stable foam. The foam was crystallized via trituration with diethylether. The crystals of the fumaric acid salt of (S)—N-(5-fluoro-4-((cis-4-fluorocyclohexyl)oxy)-2-methoxybenzyl)pyrrolidine-2-carboxamide were filtered off, washed with diethylether and dried in vacuum. Yield: 433 mg (0.88 mmol, 100%).

¹H NMR (600 MHz, methanol-d₄): δ=1.68-1.82 (m, 4H), 1.85-1.93 (m, 2H), 1.94-2.08 (m, 5H), 2.36-2.44 (m, 1H), 3.39 (dt, J=11.3, 7.0 Hz, 1H), 3.82 (s, 3H), 4.22 (dd, J=8.5, 7.0 Hz, 1H), 4.29-4.38 (m, 2H), 4.41 (tt, J=7.3, 3.4 Hz, 1H), 4.66 (ddt, J=48.7, 6.9, 3.6 Hz, 1H), 6.69 (s, 2H), 6.74 (d, J=6.9 Hz, 1H), 7.02 (d, J=11.4 Hz, 1H).

¹⁹F NMR (471 MHz, methanol-d₄): δ=−144.98 (dd, J=11.7, 7.4 Hz).

LC-MS (M/Z [M+H]⁺): 369.30

### Example 17

### (S)—N-(5-Fluoro-4-((trans-4-fluorocyclohexyl)oxy)-2-methoxybenzyl)pyrrolidine-2-carboxamide and its Fumaric Acid Addition Salt

The trans-product was prepared according to example 16 starting from trans-4-fluorocyclohexanol (see example 16) and 4,5-difluoro-2-methoxybenzonitrile. After nitrile reduction, coupling with BOC-L-proline, deprotection and salt formation the desired product (S)—N-(5-fluoro-4-((trans-4-fluorocyclohexyl)oxy)-2-methoxybenzyl)-pyrrolidine-2-carboxamide fumaric acid salt was obtained.

¹H NMR (500 MHz, methanol-d₄): δ=1.65-1.77 (m, 4H), 1.92-2.11 (m, 7H), 2.39 (ddt, J=12.8, 8.2, 6.1 Hz, 1H), 3.39 (dt, J=11.4, 7.1 Hz, 1H), 3.82 (s, 3H), 4.21 (dd, J=8.4, 6.9 Hz, 1H), 4.28-4.39 (m, 2H), 4.51 (dd, J=6.6, 3.4 Hz, 1H), 4.67-4.80 (m, 1H), 6.69 (s, 2H), 6.74 (d, J=7.0 Hz, 1H), 7.01 (d, J=11.5 Hz, 1H).

¹⁹F NMR (471 MHz, methanol-d₄): δ−145.36 (dd, J=11.3, 6.9 Hz).

LC-MS (M/Z [M+H]⁺): 369.30

### Example 18

### (2S)—N-[[4-(4,4-Difluorocyclohexoxy)-5-fluoro-2-methoxy-phenyl]methyl]-pyrrolidine-2-carboxamide and its Fumaric Acid Addition Salt

Potassium 2-methylpropan-2-olate (1.704 g, 15.19 mmol) was suspended in THF (40 mL) and the mixture was cooled to 0° C. 4,4-Difluorocyclohexanol (2.23 g, 16.38 mmol) was added and the solution was stirred at 0° C. for 30 min. The temperature was lowered to −78° C. followed by dropwise addition of 2,4,5-trifluorobenzonitrile (2.339 g, 14.89 mmol) in THF (25 mL) at −78° C. under nitrogen. The solution was stirred at −78° C. for 2 h and warmed to room temperature over 1.5 h. Water (200 mL) was added to the reaction mixture, extracted with EtOAc (100 mL*3), the combined organic phases were washed with water and brine, dried over sodium sulfate and the solvent was removed in vacuum. The residue was purified by flash chromatography on silica (heptane/EtOAc:95/5% to 52/48%). 4-((4,4-Difluorocyclohexyl)oxy)-2,5-difluorobenzonitrile was obtained as viscous oil (3.08 g; yield 76%).

Potassium 2-methylpropan-2-olate (2.070 g, 18.45 mmol) was suspended in THF (22 mL) and the mixture was cooled to 0° C. Methanol (0.591 g, 18.45 mmol, 18.45 mmol, 0.75 mL) was added and the solution was stirred at 0° C. for 30 min. The temperature was lowered to −50° C. followed by dropwise addition of 4-((4,4-difluorocyclo-hexyl)oxy)-2,5-difluorobenzonitrile (3.6 g, 13.18 mmol) in THF (40 mL) at −50° C. under nitrogen. The solution was stirred at −50° C. for 1 h and warmed to room temperature overnight. Water (300 mL) was added to the reaction mixture, extracted with EtOAc (100 mL*3), the combined organic phases were washed with water and brine, dried over sodium sulfate and the solvent was removed in vacuum. The residue was purified by flash chromatography on silica (heptane/EtOAc). 4-((4,4-Difluorocyclohexyl)oxy)-5-fluoro-2-methoxybenzonitrile was obtained as white foam (2.54 g; yield 68%). Nitrile reduction, peptide coupling, deprotection and subsequent salt formation were performed as described in example 16. (2S)—N-[[4-(4,4-difluorocyclohexoxy)-5-fluoro-2-methoxy-phenyl]methyl]pyrrolidine-2-carboxamide fumarate was obtained as white hard foam.

LC-MS (M/Z [M+H]⁺): 387.2

¹H NMR (600 MHz, methanol-d₄) δ 7.03 (d, J=11.5 Hz, 1H), 6.78 (d, J=6.9 Hz, 1H), 6.69 (s, 2H), 4.58 (dt, J=6.0, 3.0 Hz, 1H), 4.36, 4.31 (ABq, J=14.8 Hz, 2H), 4.23 (dd, J=8.4, 6.9 Hz, 1H), 3.83 (s, 3H), 3.43-3.35 (m, 1H), 3.35-3.28 (m, 1H), 2.45-2.36 (m, 1H), 2.19-2.07 (m, 2H), 2.09-2.01 (m, 2H), 2.03-1.86 (m, 7H).

### Example 19

### (2S)—N-[[5-Fluoro-2-methoxy-4-(3,3,3-trifluoropropoxy)phenyl]methyl]pyrrolidine-2-carboxamide and its Fumaric Acid Addition Salt

(S)-tert-Butyl 2-((5-fluoro-4-hydroxy-2-methoxybenzyl)carbamoyl)pyrrolidine-1-carboxylate (400 mg, 1.086 mmol, see example 1), triphenylphosphine (575 mg, 2.172 mmol) and 3,3,3-trifluoropropan-1-ol (0.144 mL, 1.629 mmol) were dissolved in THF (12 mL) under inert atmosphere. The reaction mixture was cooled to −10° C. and DIAD (0.444 mL, 2.172 mmol) was added dropwise via syringe and stirring at −10° C. continued for 2 h. Subsequently the cooling bath was removed and the reaction mixture stirred at room temperature over the weekend. Since the reaction was not complete, additional triphenylphosphine (575 mg, 2.172 mmol), 3,3,3-trifluoropropan-1-ol (0.144 mL, 1.629 mmol) and DIAD (0.444 mL, 2.172 mmol) were added to the reaction mixture at −5° C. and the reaction stirred at room temperature overnight. Since the reaction was still in-complete, once again DIAD (0.444 mL, 2.172 mmol) was added at −5° C. and the reaction stirred at room temperature overnight. Water was added, the reaction mixture extracted three times with ethyl acetate, the organic phases were combined and dried over sodium sulfate. Evaporation of the solvent and purification of the raw material by column chromatography resulted in (S)-tert-butyl 2-((5-fluoro-2-methoxy-4-(3,3,3-trifluoropropoxy)benzyl)carbamoyl)pyrrolidine-1-carboxylate (180 mg; yield 37%) BOC deprotection and subsequent salt formation was performed as described in example 1 resulting in (2S)—N-[[5-fluoro-2-methoxy-4-(3,3,3-trifluoropropoxy)phenyl]-methyl]pyrrolidine-2-carboxamide fumarate as white powder after lyophilization.

LC-MS (M/Z [M+H]⁺): 365.2

¹H NMR (500 MHz, methanol-d₄) δ 7.03 (d, J=11.5 Hz, 1H), 6.74 (d, J=7.0 Hz, 1H), 6.68 (s, 2H), 4.36, 4.31 (ABq, J=14.8 Hz, 2H), 4.30 (t, J=6.1 Hz, 2H), 4.23 (dd, J=8.4, 6.8 Hz, 1H), 3.85 (s, 3H), 3.39 (dt, J=11.4, 7.0 Hz, 1H), 3.35-3.27 (m, 1H), 2.76-2.63 (m, 2H), 2.45-2.34 (m, 1H), 2.08-1.92 (m, 3H).

### Example 20

### (2S)—N-[[5-Fluoro-2-methoxy-4-(2,2,3,3-tetrafluoropropoxy)phenyl]methyl]-pyrrolidine-2-carboxamide and its Hydrochloride

The compound was prepared according to example 19 by Mitsunobu reaction starting from 2,2,3,3-tetrafluoropropan-1-ol and (S)-tert-butyl 2-((5-fluoro-4-hydroxy-2-methoxybenzyl)carbamoyl)pyrrolidine-1-carboxylate. After BOC deprotection and salt formation the desired product (2S)—N-[[5-fluoro-2-methoxy-4-(2,2,3,3-tetrafluoropropoxy)phenyl]methyl]pyrrolidine-2-carboxamide hydrochloride salt was obtained as white powder after lyophilization.

LC-MS (M/Z [M+H]⁺): 383.2

¹H NMR (500 MHz, methanol-d₄) δ 7.04 (d, J=11.5 Hz, 1H), 6.82 (d, J=7.0 Hz, 1H), 6.31 (tt, J=52.6, 5.3 Hz, 1H), 4.54 (tt, J=12.5, 1.6 Hz, 2H), 4.34, 4.30 (ABq, J=14.9 Hz, 2H), 3.96-3.89 (m, 1H), 3.85 (s, 3H), 3.22-3.13 (m, 1H), 3.13-3.06 (m, 1H), 2.31-2.21 (m, 1H), 1.93-1.81 (m, 3H).

### Example 21

### (2S)—N-[[5-Fluoro-4-(2,2,3,4,4,4-hexafluorobutoxy)-2-methoxy-phenyl]methyl]-pyrrolidine-2-carboxamide and its 2,2,2-trifluoroacetic Acid Addition Salt

The compound was prepared according to example 11 by Mitsunobu reaction starting from 2,2,3,4,4,4-hexafluorobutan-1-ol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-4-(2,2,3,4,4,4-hexafluorobutoxy)-2-methoxy-phenyl]methyl]pyrrolidine-2-carboxamide was obtained as 2,2,2-trifluoroacetic acid salt.

MS(APCI+) (M/Z [M+H]⁺): 433

¹H NMR (400 MHz, DMSO-d₆) δ 7.06 (d, J=11.8 Hz, 1H), 6.95 (d, J=7.1 Hz, 1H), 4.76 (m, 2H), 4.68 (m, 1H), 4.23 (s, 2H), 4.04 (m, 1H), 3.82 (m, 3H), 3.14 (m, 2H), 2.24 (m, 1H), 1.80 (m, 3H).

### Example 22

### (2S)—N-[[5-Fluoro-2-methoxy-4-(2,2,3,3-tetrafluoro-1-methyl-propoxy)phenyl]-methyl]pyrrolidine-2-carboxamide and its 2,2,2-trifluoroacetic Acid Addition Salt

The compound was prepared according to example 11 by Mitsunobu reaction starting from 3,3,4,4-tetrafluorobutan-2-ol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-(2,2,3,3-tetrafluoro-1-methyl-propoxy)phenyl]methyl]pyrrolidine-2-carboxamide was obtained as 2,2,2-trifluoroacetic acid salt.

MS(APCI+) (M/Z [M+H]⁺): 397

¹H NMR (400 MHz, DMSO-d₆) δ 7.08 (d, J=11.5 Hz, 1H), 6.93 (d, J=7.1 Hz, 1H), 6.77-6.42 (m, 1H), 5.06 (dd, J=14.1, 7.1 Hz, 1H), 4.24 (s, 2H), 4.17 (t, J=7.8 Hz, 1H), 3.81 (s, 3H), 3.33-3.10 (m, 2H), 2.39-2.23 (m, 1H), 2.05-1.79 (m, 3H), 1.41 (d, J=6.4 Hz, 3H).

### Example 23

### (2S)—N-[[5-Fluoro-2-methoxy-4-(4,4,4-trifluorobutoxy)phenyl]methyl]pyrrolidine-2-carboxamide and its 2,2,2-trifluoroacetic Acid Addition Salt

The compound was prepared according to example 11 by Mitsunobu reaction starting from 4,4,4-trifluorobutan-1-ol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-(4,4,4-trifluorobutoxy)phenyl]-methyl]-pyrrolidine-2-carboxamide was obtained as 2,2,2-trifluoroacetic acid salt.

MS(APCI+) (M/Z [M+H]⁺): 379

¹H NMR (400 MHz, DMSO-d₆) δ 7.04 (d, J=11.8 Hz, 1H), 6.82 (d, J=7.2 Hz, 1H), 4.78 (p, J=6.2 Hz, 2H), 4.23 (s, 2H), 4.19-4.11 (m, 3H), 3.34-3.14 (m, 2H), 2.49-2.23 (m, 3H), 2.04-1.73 (m, 5H).

### Example 24

### (2S)—N-[[5-Fluoro-2-methoxy-4-(3,3,4,4,4-pentafluorobutoxy)phenyl]methyl]-pyrrolidine-2-carboxamide and its 2,2,2-trifluoroacetic Acid Addition Salt

The compound was prepared according to example 11 by Mitsunobu reaction starting from 3,3,4,4,4-pentafluorobutan-1-ol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-(3,3,4,4,4-pentafluorobutoxy)phenyl]-methyl]pyrrolidine-2-carboxamide was obtained as 2,2,2-trifluoroacetic acid salt.

MS(APCI+) (M/Z [M+H]⁺): 415

¹H NMR (400 MHz, DMSO-d₆) δ 7.05 (d, J=11.8 Hz, 1H), 6.86 (d, J=7.3 Hz, 1H), 4.38 (t, J=5.7 Hz, 2H), 4.24 (s, 2H), 4.17 (t, J=7.9 Hz, 1H), 3.23 (ddt, J=30.9, 11.3, 7.1 Hz, 2H), 2.74 (tt, J=18.8, 5.6 Hz, 2H), 2.38-2.27 (m, 1H), 1.97-1.80 (m, 3H).

### Example 25

### (2S)—N-[[5-Fluoro-2-methoxy-4-(4,4,4-trifluoro-2-methyl-butoxy)phenyl]methyl]-pyrrolidine-2-carboxamide and its 2,2,2-trifluoroacetic Acid Addition Salt

The compound was prepared according to example 11 by Mitsunobu reaction starting from 4,4,4-trifluoro-2-methylbutan-1-ol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-(4,4,4-trifluoro-2-methyl-butoxy)phenyl]methyl]pyrrolidine-2-carboxamide was obtained as 2,2,2-trifluoroacetic acid salt.

MS(APCI+) (M/Z [M+H]⁺): 393

¹H NMR (400 MHz, DMSO-d₆) δ 7.04 (d, J=11.7 Hz, 1H), 6.81 (d, J=7.3 Hz, 1H), 4.86-4.72 (m, 1H), 4.24 (d, J=4.0 Hz, 2H), 4.17 (t, J=7.8 Hz, 1H), 3.99 (qd, J=9.6, 5.2 Hz, 2H), 3.80 (s, 3H), 3.33-3.11 (m, 2H), 2.39-2.19 (m, 3H), 2.00-1.75 (m, 3H), 1.11 (d, J=5.9 Hz, 3H).

### Example 26

### (2S)—N-[[4-[(2,2-Difluorocyclopropyl)methoxy]-5-fluoro-2-methoxy-phenyl]-methyl]pyrrolidine-2-carboxamide and its 2,2,2-trifluoroacetic Acid Addition Salt

The compound was prepared according to example 11 by Mitsunobu reaction starting from (2,2-difluorocyclopropyl)methanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[4-[(2,2-difluorocyclopropyl)methoxy]-5-fluoro-2-methoxy-phenyl]methyl]pyrrolidine-2-carboxamide was obtained as 2,2,2-trifluoroacetic acid salt.

MS(APCI+) (M/Z [M+H]⁺): 359

¹H NMR (400 MHz, DMSO-d₆) δ 7.04 (d, J=11.8 Hz, 1H), 6.82 (d, J=7.2 Hz, 1H), 4.30 (ddd, J=10.1, 6.2, 3.3 Hz, 1H), 4.23 (s, 2H), 4.21-4.13 (m, 1H), 4.04 (dd, J=10.8, 9.0 Hz, 1H), 3.81 (s, 3H), 3.23 (ddt, J=30.6, 11.4, 7.2 Hz, 2H), 2.32 (ddd, J=12.5, 8.1, 6.2 Hz, 1H), 2.27-2.14 (m, 1H), 2.04-1.86 (m, 2H), 1.86-1.78 (m, 1H), 1.80-1.68 (m, 1H), 1.45 (dtd, J=13.7, 7.8, 4.1 Hz, 1H).

### Example 27

### (2S)—N-[[5-Fluoro-2-methoxy-4-[[1-(trifluoromethyl)cyclopropyl]methoxy]phenyl]-methyl]pyrrolidine-2-carboxamide and its Hydrochloride

The compound was prepared according to example 19 by Mitsunobu reaction starting from (1-(trifluoromethyl)cyclopropyl)methanol and (S)-tert-butyl 2-((5-fluoro-4-hydroxy-2-methoxybenzyl)carbamoyl)pyrrolidine-1-carboxylate. After BOC deprotection and salt formation the desired product (2S)—N-[[5-fluoro-2-methoxy-4-[[1-(trifluoromethyl)cyclopropyl]methoxy]phenyl]methyl]pyrrolidine-2-carboxamide hydrochloride salt was obtained as white powder.

LC-MS (M/Z [M+H]⁺): 391.2

¹H NMR (500 MHz, Methanol-d₄) δ 6.97 (d, J=11.6 Hz, 1H), 6.71 (d, J=7.0 Hz, 1H), 4.30 (d, J=1.9 Hz, 2H), 4.18 (s, 2H), 3.83 (s, 3H), 3.83-3.78 (m, 1H), 3.14-3.06 (m, 1H), 3.03 (dt, J=10.9, 6.7 Hz, 1H), 2.27-2.12 (m, 1H), 1.88-1.76 (m, 3H), 1.15-1.05 (m, 2H), 1.05-0.95 (m, 2H).

### Example 28

### (2S)—N-[[4-(3,3-Difluorocyclobutoxy)-5-fluoro-2-methoxy-phenyl]methyl]-pyrrolidine-2-carboxamide and its Hydrochloride

The compound was prepared according to example 19 by Mitsunobu reaction starting from 3,3-difluorocyclobutanol and (S)-tert-butyl 2-((5-fluoro-4-hydroxy-2-methoxybenzyl)carbamoyl)pyrrolidine-1-carboxylate. After BOC deprotection and salt formation the desired product (2S)—N-[[4-(3,3-difluorocyclobutoxy)-5-fluoro-2-methoxy-phenyl]methyl]pyrrolidine-2-carboxamide hydrochloride salt was obtained as white powder after lyophilization.

LC-MS (M/Z [M+H]⁺): 359.2

¹H NMR (500 MHz, methanol-d₄) δ 7.01 (d, J=11.5 Hz, 1H), 6.59 (d, J=7.0 Hz, 1H), 4.84-4.76 (m, 1H), 4.33, 4.29 (ABq, J=14.9 Hz, 2H), 4.03-3.96 (m, 1H), 3.84 (s, 3H), 3.27-3.19 (m, 1H), 3.19-3.04 (m, 3H), 2.81-2.66 (m, 2H), 2.36-2.24 (m, 1H), 1.97-1.83 (m, 3H).

### Example 29

### (2S)—N-[[4-[(3,3-Difluorocyclobutyl)methoxy]-5-fluoro-2-methoxy-phenyl]methyl]-pyrrolidine-2-carboxamide and its Fumaric Acid Addition Salt

The compound was prepared as described in example 1. (S)-tert-Butyl 2-((5-fluoro-4-hydroxy-2-methoxybenzyl)carbamoyl)pyrrolidine-1-carboxylate (300 mg, 0.814 mmol) was dissolved in DMF (10 mL), cesium carbonate (318 mg, 0.977 mmol) was added and the reaction mixture stirred for 15 min at 50° C. (3,3-difluorocyclobutyl)-methyl 4-methylbenzenesulfonate (247 mg, 0.896 mmol) was added and the reaction mixture stirred for 30 min at 50° C. and subsequently overnight at room temperature. Water was added, the reaction mixture extracted three times with ethyl acetate, the organic phases were combined and dried over sodium sulfate. Evaporation of the solvent and purification of the raw material by column chromatography resulted in (S)-tert-butyl 2-((4-((3,3-difluorocyclobutyl)methoxy)-5-fluoro-2-methoxybenzyl)carbamoyl)-pyrrolidine-1-carboxylate as white foam (363 mg; 94% yield). After BOC deprotection and salt formation the desired product (2S)—N-[[4-[(3,3-difluorocyclobutyl)methoxy]-5-fluoro-2-methoxy-phenyl]methyl]pyrrolidine-2-carboxamide fumaric acid salt was obtained as white powder after lyophilization.

LC-MS (M/Z [M+H]⁺): 373.1

¹H NMR (600 MHz, methanol-d₄) δ 7.01 (d, J=11.5 Hz, 1H), 6.74 (d, J=7.0 Hz, 1H), 6.68 (s, 2H), 4.35, 4.31 (ABq, J=14.8 Hz, 2H), 4.20 (dd, J=8.5, 6.9 Hz, 1H), 4.11 (d, J=6.2 Hz, 2H), 3.84 (s, 3H), 3.39 (dt, J=11.3, 7.1 Hz, 1H), 3.33-3.26 (m, 1H), 2.76-2.65 (m, 2H), 2.65-2.56 (m, 1H), 2.56-2.43 (m, 2H), 2.43-2.34 (m, 1H), 2.07-1.93 (m, 3H).

### Example 30

### (2S)—N-[[5-Fluoro-2-methoxy-4-[(2,2,3,3-tetrafluorocyclobutyl)methoxy]phenyl]-methyl]pyrrolidine-2-carboxamide and its 2,2,2-trifluoroacetic Acid Addition Salt

The compound was prepared according to example 11 by Mitsunobu reaction starting from (2,2,3,3-tetrafluorocyclobutyl)methanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-[(2,2,3,3-tetrafluorocyclobutyl)-methoxy]phenyl]methyl]pyrrolidine-2-carboxamide was obtained as 2,2,2-trifluoroacetic acid salt.

MS(APCI+) (M/Z [M+H]⁺): 409

¹H NMR (400 MHz, DMSO-d₆) δ 7.05 (d, J=11.7 Hz, 1H), 6.85 (d, J=7.1 Hz, 1H), 4.38-4.25 (m, 2H), 4.23 (s, 2H), 4.15 (t, J=7.8 Hz, 1H), 3.82 (s, 3H), 3.36 (s, 1H), 3.22 (ddt, J=29.1, 11.3, 7.1 Hz, 2H), 2.85 (td, J=15.4, 7.2 Hz, 1H), 2.31 (ddd, J=14.8, 12.5, 6.9 Hz, 1H), 1.98-1.72 (m, 3H).

### Example 31

### (2S)—N-[[4-[(3,3-Difluorocyclopentyl)methoxy]-5-fluoro-2-methoxy-phenyl]methyl]-pyrrolidine-2-carboxamide and its Fumaric Acid Addition Salt

The compound was prepared according to example 19 by Mitsunobu reaction starting from (3,3-difluorocyclopentyl)methanol and (S)-tert-butyl 2-((5-fluoro-4-hydroxy-2-methoxybenzyl)carbamoyl)pyrrolidine-1-carboxylate. After BOC deprotection and salt formation the desired product (2S)—N-[[4-[(3,3-difluorocyclopentyl)methoxy]-5-fluoro-2-methoxy-phenyl]methyl]pyrrolidine-2-carboxamide fumaric acid salt was obtained as white powder after lyophilization.

LC-MS (M/Z [M+H]⁺): 387.2

¹H NMR (500 MHz, methanol-d₄) δ 7.01 (d, J=11.5 Hz, 1H), 6.72 (d, J=7.1 Hz, 1H), 6.69 (s, 2H), 4.35, 4.30 (ABq, J=14.8 Hz, 2H), 4.22 (dd, J=8.4, 6.8 Hz, 1H), 4.07-3.97 (m, 2H), 3.84 (s, 3H), 3.39 (dt, J=11.4, 7.1 Hz, 1H), 3.35-3.26 (m, 1H), 2.69-2.56 (m, 1H), 2.45-2.34 (m, 1H), 2.34-2.25 (m, 1H), 2.25-2.13 (m, 1H), 2.13-1.91 (m, 6H), 1.77-1.64 (m, 1H).

### Example 32

### (2S)—N-[[4-[(4,4-Difluorocyclohexyl)methoxy]-5-fluoro-2-methoxy-phenyl]methyl]-pyrrolidine-2-carboxamide and its Fumaric Acid Addition Salt

The compound was prepared according to example 19 by Mitsunobu reaction starting from (4,4-difluorocyclohexyl)methanol and (S)-tert-butyl 2-((5-fluoro-4-hydroxy-2-methoxybenzyl)carbamoyl)pyrrolidine-1-carboxylate. After BOC deprotection and salt formation the desired product (2S)—N-[[4-[(4,4-difluorocyclohexyl)methoxy]-5-fluoro-2-methoxy-phenyl]methyl]pyrrolidine-2-carboxamide fumaric acid salt was obtained as white powder after lyophilization.

LC-MS (M/Z [M+H]⁺): 401.2

¹H NMR (500 MHz, DMSO-d₆) δ 8.66 (t, J=5.8 Hz, 1H), 7.00 (d, J=11.9 Hz, 1H), 6.80 (d, J=7.3 Hz, 1H), 6.52 (s, 2H), 4.19 (d, J=5.8 Hz, 2H), 4.07-4.00 (m, 1H), 3.96 (d, J=6.2 Hz, 2H), 3.81 (s, 3H), 3.12-3.05 (m, 2H), 2.23-2.13 (m, 1H), 2.09-1.99 (m, 2H), 1.94-1.84 (m, 4H), 1.83-1.71 (m, 4H), 1.39-1.27 (m, 2H).

### Example 33

### (S)—N-(5-Fluoro-4-((trans-4-trifluoromethyl-cyclohexyl)oxy)-2-methoxybenzyl)-pyrrolidine-2-carboxamide and its Fumaric Acid Addition Salt

The compound was prepared according to example 19 by Mitsunobu reaction starting from cis 4-(trifluoromethyl)cyclohexanol and (S)-tert-butyl 2-((5-fluoro-4-hydroxy-2-methoxybenzyl)carbamoyl)pyrrolidine-1-carboxylate. After BOC deprotection and salt formation the desired product (S)—N-(5-fluoro-4-((trans-4-trifluoromethyl-cyclohexyl)oxy)-2-methoxybenzyl)pyrrolidine-2-carboxamide fumaric acid salt was obtained as white powder.

LC-MS (M/Z [M+H]⁺): 419.2

¹H NMR (500 MHz, methanol-d₄) δ 7.00 (d, J=11.6 Hz, 1H), 6.74 (d, J=6.9 Hz, 1H), 6.70 (s, 2H), 4.36, 4.31 (d, J=14.7 Hz, 2H), 4.28-4.19 (m, 2H), 3.83 (s, 3H), 3.40 (dt, J=11.3, 7.1 Hz, 1H), 3.34-3.29 (m, 1H), 2.45-2.34 (m, 1H), 2.25-2.14 (m, 3H), 2.09-1.93 (m, 5H), 1.56-1.41 (m, 4H).

### Example 34

### (S)—N-(5-Fluoro-4-((cis-4-trifluoromethyl-cyclohexyl)oxy)-2-methoxybenzyl)-pyrrolidine-2-carboxamide and its 2,2,2-trifluoroacetic Acid Addition Salt

The compound was prepared according to example 19 by Mitsunobu reaction starting from trans 4-(trifluoromethyl)cyclohexanol and (S)-tert-butyl 2-((5-fluoro-4-hydroxy-2-methoxybenzyl)carbamoyl)pyrrolidine-1-carboxylate. After BOC deprotection with TFA the desired product (S)—N-(5-fluoro-4-((cis-4-trifluoromethyl-cyclohexyl)oxy)-2-methoxybenzyl)pyrrolidine-2-carboxamide 2,2,2-trifluoroacetic acid salt was obtained as off white foam after lyophilization.

LC-MS (M/Z [M+H]⁺): 419.2

¹H NMR (600 MHz, methanol-d₄) δ 7.02 (d, J=11.4 Hz, 1H), 6.74 (d, J=7.0 Hz, 1H), 4.66-4.62 (m, 1H), 4.36, 4.32 (ABq, J=14.7 Hz, 2H), 4.20 (dd, J=8.5, 6.9 Hz, 1H), 3.82 (s, 3H), 3.42-3.36 (m, 1H), 3.31-3.26 (m, 1H), 2.44-2.35 (m, 1H), 2.28-2.18 (m, 1H), 2.09 (dd, J=13.8, 3.4 Hz, 2H), 2.07-1.94 (m, 3H), 1.82-1.69 (m, 4H), 1.68-1.59 (m, 2H).

### Example 35

### (2S)—N-[[5-Fluoro-2-methoxy-4-[3-(trifluoromethyl)cyclohexoxy]phenyl]methyl]-pyrrolidine-2-carboxamide and its 2,2,2-trifluoroacetic Acid Addition Salt

The compound was prepared according to example 11 by Mitsunobu reaction starting from 3-(trifluoromethyl)cyclohexanol and (S)-tert-butyl 2-((5-fluoro-4-hydroxy-2-methoxybenzyl)carbamoyl)pyrrolidine-1-carboxylate. After BOC deprotection with HCl and purification of the raw material by reverse phase HPLC (TFA method) the desired product (2S)—N-[[5-fluoro-2-methoxy-4-[3-(trifluoromethyl)cyclohexoxy]phenyl]methyl]pyrrolidine-2-carboxamide was obtained as 2,2,2-trifluoroacetic acid salt.

MS(APCI+) (M/Z [M+H]⁺): 419

¹H NMR (400 MHz, DMSO-d₆) δ 7.04 (d, J=11.8 Hz, 1H), 6.82 (d, J=7.2 Hz, 1H), 4.47-4.35 (m, 1H), 4.23 (s, 2H), 4.17 (t, J=7.9 Hz, 1H), 3.79 (s, 3H), 3.23 (ddt, J=30.9, 11.5, 7.1 Hz, 2H), 2.33 (ddd, J=15.0, 12.3, 6.4 Hz, 1H), 2.19 (d, J=11.8 Hz, 1H), 2.08 (d, J=11.3 Hz, 1H), 1.99-1.75 (m, 5H), 1.69-1.24 (m, 5H).

### Example 36

### (2S)—N-[[5-Fluoro-2-methoxy-4-(2,2,3,3-tetrafluoropropoxy)phenyl]methyl]-1-methyl-pyrrolidine-2-carboxamide and its Hydrochloride

(S)—N-(5-Fluoro-2-methoxy-4-(2,2,3,3-tetrafluoropropoxy)benzyl)pyrrolidine-2-carboxamide (22 mg, 0.058 mmol; example 20) was dissolved in DCE (2 mL) under nitrogen and formaldehyde (0.434 mL, 5.75 mmol) and sodium triacetoxyborohydride (31.4 mg, 0.144 mmol) were added. The reaction mixture was stirred at room temperature overnight. Subsequently EtOAc and saturated NaHCO₃-solution were added, the organic layer was separated, the NaHCO₃-layer was washed twice with EtOAc and the combined organic layers were dried with sodium sulfate, filtered and concentrated. The compound was purified by flash chromatography to obtain (2S)—N-[[5-fluoro-2-methoxy-4-(2,2,3,3-tetrafluoropropoxy)phenyl]methyl]-1-methyl-pyrrolidine-2-carboxamide as white foam (17 mg, yield 67%). One equivalent hydrochloric acid and water were added and the resulting solution lyophilized to obtain (2S)—N-[[5-fluoro-2-methoxy-4-(2,2,3,3-tetrafluoropropoxy)phenyl]methyl]-1-methyl-pyrrolidine-2-carboxamide hydrochloride as white powder.

LC-MS (M/Z [M+H]⁺): 397.2

¹H NMR (500 MHz, methanol-d₄) δ 7.02 (d, J=11.5 Hz, 1H), 6.82 (d, J=6.9 Hz, 1H), 6.31 (tt, J=52.6, 5.2 Hz, 1H), 4.54 (tt, J=12.5, 1.7 Hz, 2H), 4.34, 4.30 (ABq, J=15.0 Hz, 2H), 3.85 (s, 3H), 3.27-3.21 (m, 1H), 3.13 (br s, 1H), 2.60-2.51 (m, 1H), 2.47 (s, 3H), 2.33-2.22 (m, 1H), 1.95-1.79 (m, 3H).

### Example 37

### (2S)—N-[[4-(3,3-Difluorocyclobutoxy)-5-fluoro-2-methoxy-phenyl]methyl]-1-methyl-pyrrolidine-2-carboxamide and its Hydrochloride

The compound was prepared according to example 36 by reductive amination starting from (S)—N-(4-(3,3-difluorocyclobutoxy)-5-fluoro-2-methoxybenzyl)-1-methyl-pyrrolidine-2-carboxamide (example 28) and formaldehyde. The desired product (2S)—N-[[4-(3,3-difluorocyclobutoxy)-5-fluoro-2-methoxy-phenyl]methyl]-1-methyl-pyrrolidine-2-carboxamide hydrochloride salt was obtained as white powder after lyophilization.

LC-MS (M/Z [M+H]⁺): 373.2

¹H NMR (500 MHz, methanol-d₄) δ 6.98 (d, J=11.5 Hz, 1H), 6.59 (d, J=7.0 Hz, 1H), 4.84-4.76 (m, 1H), 4.32, 4.28 (ABq, J=14.9 Hz, 2H), 3.84 (s, 3H), 3.28-3.20 (m, 1H), 3.17-3.04 (m, 3H), 2.82-2.66 (m, 2H), 2.54 (q, J=8.5 Hz, 1H), 2.46 (s, 3H), 2.33-2.20 (m, 1H), 1.94-1.78 (m, 3H).

### Example 38

### (2S)—N-[[4-[(3,3-Difluorocyclopentyl)methoxy]-5-fluoro-2-methoxy-phenyl]methyl]-1-methyl-pyrrolidine-2-carboxamide and its Fumaric Acid Addition Salt

The compound was prepared according to example 36 by reductive amination starting from (2S)—N-[[4-[(3,3-difluorocyclopentyl)methoxy]-5-fluoro-2-methoxy-phenyl]methyl]pyrrolidine-2-carboxamide (example 31) and formaldehyde. The desired product (2S)—N-[[4-[(3,3-difluorocyclopentyl)methoxy]-5-fluoro-2-methoxy-phenyl]methyl]-1-methyl-pyrrolidine-2-carboxamide fumaric acid salt was obtained as white powder after lyophilization.

LC-MS (M/Z [M+H]⁺): 401.2

¹H NMR (500 MHz, methanol-d₄) δ 7.00 (d, J=11.5 Hz, 1H), 6.73 (d, J=7.0 Hz, 1H), 6.70 (s, 2H), 4.36, 4.31 (ABq, J=14.7 Hz, 2H), 4.07-3.97 (m, 2H), 3.84 (s, 3H), 3.79 (dd, J=9.1, 6.9 Hz, 1H), 3.62-3.54 (m, 1H), 3.02 (dt, J=11.0, 8.3 Hz, 1H), 2.79 (s, 3H), 2.68-2.57 (m, 1H), 2.53-2.41 (m, 1H), 2.37-2.25 (m, 1H), 2.25-1.91 (m, 7H), 1.77-1.64 (m, 1H).

### Example 39

### (2S)—N-(5-Fluoro-2-methoxy-4-((trans-4-(trifluoromethyl)cyclohexyl)oxy)benzyl)-1-methyl-pyrrolidine-2-carboxamide and its Fumaric Acid Addition Salt

The compound was prepared according to example 36 by reductive amination starting from (S)—N-(5-fluoro-4-((trans-4-trifluoromethyl-cyclohexyl)oxy)-2-methoxybenzyl)pyrrolidine-2-carboxamide (example 33) and formaldehyde. The desired product (2S)—N-(5-fluoro-2-methoxy-4-((trans-4-(trifluoromethyl)cyclohexyl)-oxy)benzyl)-1-methyl-pyrrolidine-2-carboxamide fumaric acid salt was obtained as white powder after lyophilization.

LC-MS (M/Z [M+H]⁺): 433.2

¹H NMR (500 MHz, methanol-d₄) δ 6.99 (d, J=11.4 Hz, 1H), 6.74 (d, J=7.0 Hz, 1H), 6.70 (s, 2H), 4.36, 4.31 (ABq, J=14.7 Hz, 2H), 4.28-4.20 (m, 1H), 3.83 (s, 3H), 3.78 (dd, J=9.0, 6.8 Hz, 1H), 3.62-3.53 (m, 1H), 3.06-2.97 (m, 1H), 2.78 (s, 3H), 2.53-2.39 (m, 1H), 2.25-2.15 (m, 3H), 2.15-2.05 (m, 1H), 2.05-1.92 (m, 4H), 1.56-1.41

### Example 40

### (2S)—N-[[4-(4,4-Difluorocyclohexoxy)-5-fluoro-2-methoxy-phenyl]methyl]-1-methyl-pyrrolidine-2-carboxamide and its Fumaric Acid Addition Salt

To a mixture (4-((4,4-difluorocyclohexyl)oxy)-5-fluoro-2-methoxyphenyl)methanamine (0.225 g, 0.778 mmol; see example 18) in DMF (7.8 mL), (S)-1-methylpyrrolidine-2-carboxylic acid (0.121 g, 0.933 mmol), DIPEA (0.272 mL, 1.556 mmol) and HBTU (0.354 g, 0.933 mmol) were added at 0° C. The reaction mixture was stirred at 0° C. for 1 h and overnight at room temperature. Data LCMS showed MS of the desired product. Water was added to the reaction mixture and extracted three times with tert.-butylmethylether. The combined organic phases were washed once with water and brine, dried over Na₂SO₄, filtered and concentrated in vacuum. The residue was purified by column chromatography (EtOAc/heptane 20:80->80:20, 12 g-column, flow 12 ml/min) to obtain the desired product (2S)—N-[[4-(4,4-difluorocyclohexoxy)-5-fluoro-2-methoxy-phenyl]methyl]-1-methyl-pyrrolidine-2-carboxamide (244 mg, 78% yield) as colorless oil. (2S)—N-[[4-(4,4-difluorocyclohexoxy)-5-fluoro-2-methoxy-phenyl]methyl]-1-methyl-pyrrolidine-2-carboxamide fumaric acid salt was obtained as white foam (305 mg, yield 97%).

LC-MS (M/Z [M+H]⁺): 401.2

¹H NMR (500 MHz, DMSO-d₆) δ 8.13 (t, J=6.2 Hz, 1H), 6.91 (d, J=11.8 Hz, 1H), 6.88 (d, J=7.2 Hz, 1H), 6.61 (s, 2H), 4.66-4.58 (m, 1H), 4.23-4.10 (m, 2H), 3.79 (s, 3H), 3.07 (tt, J=6.6, 2.7 Hz, 1H), 2.87 (dd, J=9.4, 5.5 Hz, 1H), 2.38-2.28 (m, 4H), 2.16-1.89 (m, 5H), 1.89-1.80 (m, 4H), 1.80-1.64 (m, 3H)

### Example 41

### (2S)—N-[[5-Fluoro-2-methoxy-4-[trans 4-(trifluoromethyl)cyclohexoxy]phenyl]-methyl]azetidine-2-carboxamide and its 2,2,2-trifluoroacetic Acid Addition Salt

To a solution of potassium 2-methylpropan-2-olate (0.280 g, 2.498 mmol) in THF (4 mL) was added dropwise a solution of trans-4-(trifluoromethyl)cyclohexanol (0.4 g, 2.379 mmol) in THF (2 mL) at 0° C., then it was stirred for 1 hour, the resulting above solution was added dropwise to a solution of 2,4,5-trifluorobenzonitrile (0.448 g, 2.85 mmol) in THF (4 mL) at −70° C. over 40 minutes. Then the reaction mixture was stirred at −70° C. for 3 h and allowed to warm to 20° C. over 1.5 h and stirred at 20° C. for 16 h. It was quenched with water, extracted with EtOAc three times, the extracts were washed with brine, dried over Na₂SO₄, filtered, concentrated to give the crude product, which was recrystallized from PE/EtOAc (5:1) to give 2,5-difluoro-4-((trans-4-(trifluoromethyl)cyclohexyl)oxy)benzonitrile (0.480 g, 1.573 mmol, 66.1% yield) as light yellow solid.

To a solution of potassium 2-methylpropan-2-olate (0.239 g, 2.129 mmol) in THF (4 mL) was added dropwise a solution of methanol (0.0789 g, 2.457 mmol) in THF (1 mL) at 0° C., then it was stirred for 1 hour, the resulting above solution was added dropwise to a solution of 2,5-difluoro-4-(trans-4-(trifluoromethyl)cyclohexyl)oxy)-benzonitrile (0.5 g, 1.638 mmol) in THF (5 mL) at −50° C. over 30 minutes. Then the reaction mixture was stirred at −50° C. for 3 h and allowed to warm to 20° C. over 1.5 h and stirred at 20° C. for 16 h. It was quenched with water, extracted with EtOAc three times, the extracts were washed with brine, dried over Na₂SO₄, filtered, concentrated to give the crude product, which was recrystallized from PE/EtOAc (5:1) to give 5-fluoro-2-methoxy-4-((trans-4-(trifluoromethyl)cyclohexyl)oxy)benzonitrile (0.4 g, 1.261 mmol, 77% yield) as white solid.

To a solution of 5-fluoro-2-methoxy-4-((trans-4-(trifluoromethyl)cyclohexyl)-oxy)benzonitrile (0.1 g, 0.315 mmol) in MeOH (4 mL) was added Pd/C (0.1 g, 10%, wet) and hydrogen chloride (0.263 mL, 3.15 mmol), the resulting mixture was stirred at 25° C. under H₂ atmosphere (40 psi) for 16 h. Then it was filtered, concentrated under reduced pressure to give (5-fluoro-2-methoxy-4-((trans-4-(trifluoromethyl)cyclohexyl)-oxy)phenyl)methanamine hydrochloride (0.1 g, 98% yield) as light yellow solid.

In 4 ml vial a solution of (S)-1-(tert-butoxycarbonyl)azetidine-2-carboxylic acid (29 mg, 0.14 mmol) was added dissolved in N,N-dimethylacetamide (0.5 mL), followed by a solution of HATU (76 mg, 0.2 mmol) dissolved in N,N-dimethylacetamide (0.5 ml), followed by 34 μL of N,N-diisopropylethylamine neat. Then a solution of (5-fluoro-2-methoxy-4-((trans-4-(trifluoromethyl)cyclohexyl)oxy)phenyl)methanamine (31 mg, 0.1 mmol) dissolved in N,N-dimethylacetamide (1.0 μL) was added, and the reaction was shaken at room temperature for 4 hours. Then the reaction was concentrated to dryness and 1.0 mL of HCl/dioxane was added and shaken for 2 hours. After that the reaction was concentrated to dryness and was re-dissolved in 1800 μL of a 1:1 v/v solution of DMSO/MeOH, checked by LC/MS and purified by reverse phase HPLC (TFA method) to provide the desired compound (2S)—N-[[5-fluoro-2-methoxy-4-[trans 4-(trifluoromethyl)cyclohexoxy]phenyl]methyl]azetidine-2-carboxamide as 2,2,2-trifluoroacetic acid salt (29% yield).

MS(APCI+) (M/Z [M+H]⁺): 405

¹H NMR (400 MHz, DMSO-d₆/D₂O, Temp=27° C.) δ 7.03 (d, J=11.6 Hz, 1H), 6.83 (d, J=7.2 Hz, 1H), 4.98-4.80 (m, 1H), 4.45-4.29 (m, 1H), 4.22 (s, 2H), 4.07-3.92 (m, 1H), 3.81-3.72 (m, 4H), 2.74-2.60 (m, 1H), 2.46-2.23 (m, 2H), 2.23-2.06 (m, 2H), 2.03-1.82 (m, 2H), 1.52-1.34 (m, 4H).

### Example 42

### 2,3,3,4,4,5,5-Heptadeuterio-N-[[5-fluoro-2-methoxy-4-[4-(trifluoromethyl)cyclohexoxy]phenyl]methyl]pyrrolidine-2-carboxamide and its 2,2,2-trifluoroacetic Acid Addition Salt

The compound was prepared according to example 41 by peptide coupling of (5-fluoro-2-methoxy-4-((trans-4-(trifluoromethyl)cyclohexyl)oxy)phenyl)methanamine with deuterated (S)-1-(tert-butoxycarbonyl)pyrrolidine-2-carboxylic acid-D₇. After BOC deprotection with TFA and purification of the raw material by reverse phase HPLC the desired product 2,3,3,4,4,5,5-heptadeuterio-N-[[5-fluoro-2-methoxy-4-[4-(trifluoromethyl)cyclohexoxy]phenyl]methyl]pyrrolidine-2-carboxamide was obtained as 2,2,2-trifluoroacetic acid salt.

MS (ESI+) m/z [M+H]⁺: 426,30

### Example 43

### (2S)—N-[[5-Fluoro-4-[4-(fluoromethyl)cyclohexoxy]-2-methoxy-phenyl]methyl]-pyrrolidine-2-carboxamide and its 2,2,2-trifluoroacetic Acid Addition Salt

### 43.1 4-((4-(((tert-Butyldimethylsilyl)oxy)methyl)cyclohexyl)oxy)-5-fluoro-2-methoxybenzonitrile

To 185 mg sodium hydride (50% weight percent, 3.85 mmol, 2.10 eq.) in DMF (30 ml) was added 493 mg 4-((tert-butyldimethylsilyl)oxy)methyl)cyclohexanol (2.02 mmol, 1.1 eq.) under an argon atmosphere at room temperature. The reaction mixture was stirred for 10 h at room temperature. 310 mg 4,5-Difluoro-2-methoxybenzonitrile (1.833 mmol, 1.0 eq.) was added and the reaction mixture was heated to 60° C. for 2 h. The reaction mixture was allowed to cool to room temperature and water was slowly added. The reaction mixture was extracted with ethyl acetate. The combined organic phases were washed twice with water and once with brine, dried with MgSO4. The solvent was evaporated under reduced pressure. The crude product was purified by column chromatography on silica (eluent: 0-10% methanol in dichloromethane) to yield the title compound (574 mg, 80% yield).

ESI-MS: m/z (%): 394.30 (100, [M+H]⁺).

### 43.2 4-((4-(((tert-Butyldimethylsilyl)oxy)methyl)cyclohexyl)oxy)-5-fluoro-2-methoxyphenyl)methanamine

To a solution of 290 mg 4-((4-(((tert-butyldimethylsilyl)oxy)methyl)cyclohexyl)-oxy)-5-fluoro-2-methoxybenzonitrile (0.737 mmol, 1.00 eq) in THF (10 ml) was slowly added 1.1 ml of a 2 molar borane-methyl sulfide complex in THF solution (2.21 mmol, 3.00 eq.). The reaction mixture was heated in a microwave unit to 60° C. for 1 h. The reaction mixture was cooled to 0° C. and slowly aqueous ammonium chloride solution was added. The reaction mixture was extracted with dichloromethane. The organic phase was dried with MgSO4, filtrated and the solvent was evaporated under reduced pressure. The crude product was purified by column chromatography on silica (eluent: 0-10% methanol in dichloromethane) to yield the title compound (116 mg, 40% yield).

ESI-MS: m/z (%): 381.30 (100, [M-CH₃]⁺).

### 43.3 (S)-tert-Butyl 2-((4-((4-(((tert-butyldimethylsilyl)oxy)methyl)cyclohexyl)oxy)-5-fluoro-2-methoxybenzyl)carbamoyl)pyrrolidine-1-carboxylate

57.4 mg Boc-L-Proline (0.267 mmol, 1.00 eq) was dissolved in DMF (10 ml) and 43.2 mg 1,1′-carbonyldiimidazole (CDI) was added. The mixture was heated to 50° C. for 30 min. 106 mg (4-((4-(((tert-butyldimethylsilyl)oxy)methyl)cyclohexyl)oxy)-5-fluoro-2-methoxyphenyl)methanamine (0.267 mmol, 1.00 eq) was dissolved in pyridine (10 ml) and then added to the reaction mixture which was then further heated for 5 h at 80° C. and overnight at room temperature. The reaction mixture was evaporated under reduced pressure and codistilled twice with toluene. The obtained residue was partitioned between bicarbonate solution and ethyl acetate. The organic phase was separated and the aqueous phase was extracted twice with ethyl acetate. The combined organic phases were washed with bicarbonate solution, dried with MgSO4, filtrated and the solvent was evaporated under reduced pressure. The crude product was purified by column chromatography on silica (eluent: 0-10% methanol in dichloromethane) to yield the title compound (86 mg, 54% yield).

ESI-MS: m/z (%): 617.40 (100, [M+Na]⁺).

### 43.4 (S)-tert-Butyl 2-(((5-fluoro-4-((4-(hydroxymethyl)cyclohexyl)oxy)-2-methoxy-benzyl)methyl)carbamoyl)pyrrolidine-1-carboxylate

To a solution of 83 mg (S)-tert-butyl 2-((5-fluoro-4-((4-hydroxymethyl)cyclo-hexyl)oxy)-2-methoxybenzyl)carbamoyl)pyrrolidine-1-carboxylate (0.140 mmol, 1.00 eq) in THF (6 mL) was added 0.28 mL tetrabutylammonium fluoride (1M in THF, 0.28 mmol, 2.00 eq). The mixture was stirred at room temperature for 18 h. Then a saturated aqueous NH₄Cl solution was added and the mixture was extracted with ethyl acetate. The organic phase was dried over MgSO₄, filtrated and the solvent was evaporated. The crude product was purified by column chromatography on silica (eluent: 0-10% methanol in dichloromethane) to yield the title compound (81%, 0.112 mmol).

ESI-MS: m/z (%): 381.30 (100, [M-Boc+H]⁺), 481.30 (20, [M+H]⁺), 503.30 (80, [M+Na]⁺).

### 43.5 (S)-tert-Butyl 2-(((5-fluoro-2-methoxy-4-((4-(((methylsulfonyl)oxy)methyl)-cyclohexyl)oxy)benzyl)methyl)carbamoyl)pyrrolidine-1-carboxylate

A solution of 54 mg (S)-tert-butyl 2-(((5-fluoro-4-((4-(hydroxymethyl)cyclo-hexyl)oxy)-2-methoxybenzyl)methyl)carbamoyl)pyrrolidine-1-carboxylate (0.112 mmol, 1.00 eq) and 0.031 mL triethylamine (0.225 mmol, 2.00 eq) in dichloromethane (2 ml) was cooled to 0° C. and 14.2 mg methanesulfonyl chloride (0.124 mmol, 1.10 eq) was slowly added. Then the mixture was warmed to room temperature and stirred for 2 h. Then water was added and the solution was extracted with dichloromethane. The organic phase was dried over MgSO₄, filtrated, the solvent was evaporated. The crude product was purified by column chromatography on silica (eluent: 0-10% methanol in dichloromethane) to yield the title compound (30 mg, 48% yield).

ESI-MS: m/z (%): 459.30 (100, [M-Boc+H]⁺), 581.30 (90, [M+Na]⁺).

### 43.6 (S)-tert-Butyl 2-(((5-fluoro-4-((4-(fluoromethyl)cyclohexyl)oxy)-2-methoxy-benzyl)carbamoyl)pyrrolidine-1-carboxylate

A solution of 33 mg (S)-tert-butyl 2-(((5-fluoro-2-methoxy-4-((4-(((methyl-sulfonyl)oxy)methyl)cyclohexyl)oxy)benzyl)carbamoyl)pyrrolidine-1-carboxylate (0.054 mmol, 1.00 eq) and 32.6 mg cesium fluoride (0.215 mmol, 4.00 eq) in t-butanol (2 ml) was heated in the microwave at 80° C. for 18 h and at 90° C. for 32 h. Then a saturated aqueous NaHCO₃ solution was added and the mixture was extracted with dichloromethane. The combined organic phases were dried over MgSO₄ and the solvent was evaporated. The crude product was purified by column chromatography on silica (eluent: 0-10% methanol in dichloromethane) to yield the title compound (19.7 mg, 76% yield).

ESI-MS: m/z (%): 383.20 (90, [M-Boc+H]⁺), 505.20 (100, [M+Na]⁺).

### 43.7 (S)—N-((5-Fluoro-4-((4-(fluoromethyl)cyclohexyl)oxy)-2-methoxybenzyl)-pyrrolidine-2-carboxamide, Trifluoroacetate

A solution of 19.7 mg (S)-tert-butyl 2-(((5-fluoro-4-((4-(fluoromethyl)cyclo-hexyl)oxy)-2-methoxybenzyl)carbamoyl)pyrrolidine-1-carboxylate (0.041 mmol, 1.00 eq) and 0.016 mL trifluoroacetic acid (0.204 mmol, 5.00 eq) in dichloromethane (2 mL) was stirred at room temperature for 24 h. Then the solvent was evaporated and the crude product was purified via preparative HPLC to yield the title compound as TFA salt (1.4 mg, 7% yield).

ESI-MS: m/z (%): 383.20 (100, [M+H]⁺).

¹H NMR (600 MHz, CDCl₃): δ 7.40 (bs, 1H), 6.95 (d, 1H), 6.55 (d, 1H), 4.60 (m, 1H), 4.35 (m, 2H), 4.30 (m, 1H), 4.20 (m, 1H), 4.10 (m, 1H), 3.80 (s, 3H), 3.40 (m, 1H), 3.35 (m, 1H), 2.40 (m, 1H), 2.15 (m, 2H), 2.10-1.95 (m, 3H), 1.90 (m, 2H), 1.75-1.60 (m, 2H), 1.55-1.45 (m, 2H), 1.20-1.10 (m, 2H).

### II. Biological Tests

### Functional Activity

### 1. Human 5-HT_{2C} Functional Assay

The functional activity of compounds of formula I was assayed by incubation with U2OS_HTR_{2C}_β-Arrestin cells (DiscoverX, 93-0289C3) to induce beta-arrestin2 recruitment to the 5-HT_{2C} receptor. The agonist-induced recruitment and proximity of the receptor and beta-arrestin2 leads to complementation and formation of active β-galactosidase. The enzyme complementation results in enzyme activity, which is measured following the termination of the agonist incubation using DiscoveRx's detection reagent, which contains a chemiluminescent substrate which produces a high intensity signal. Cells were plated and a medium-change to a 1% serum containing medium was performed 24 h later. The next day, test compounds were added and incubated for 1.5 h before addition of detection reagent.

The response produced was measured and compared with the response produced by 10 [mu]M 5-HT or the maximal effect induced by 5-HT (defined as 100%) to which it was expressed as a percentage response (relative efficacy). Dose response curves were con-structed using Graphpad Prism (Graph Software Inc.) or using in house adapted software using a 4 parameter dose response model with variable slope (fit=(Bottom+(Top−Bottom)/(1+10\hat{ }((LogEC50−x)*Hill Slope))res=(y−fit)). Results are compiled in the table below.

### 2. Human 5-HT_{2A} Functional Assay

Functional activity on the 5-HT_{2A} receptor was determined by testing the effect of the compounds I on calcium mobilisation in CHO-K1 cells, stably transfected with human 5-HT_{2A} receptor. Cells were seeded into sterile black 384-well plates with clear bottom at 25,000 cells/well in a volume of 25 μl and grown for 5-6 hours at 37° C., in 5% CO₂ in tissue culture medium (“Ultra CHO” by LONZA), containing 1% dialysed FCS and 50 μg/ml gentamicin (Invitrogen). After this incubation, medium was replaced by a serum free version of the same tissue culture medium followed by incubation overnight at 37° C. and in 5% CO₂. Cells were then loaded with a fluorescent calcium-sensitive dye in the presence of 0.07% probenecid for an hour at 37° C., according to the manufacturer's protocol (Ca5-Assay Kit, Molecular Devices), followed by an additional 60 min incubation at room temperature. Serial compound dilutions (final concentrations of 10⁻¹⁰ to 10⁻⁵M, prepared in HBSS+50 mM HEPES) were first added to the cells alone (“first addition” to assess agonism on the 5-HT_{2A} receptor), then after 8 min, serotonin was added to the same wells at a final concentration of 3×10⁻⁸ M (“second addition” to see potential antagonistic effect) and the maximal calcium response was determined using a FLIPR® Tetra instrument (Molecular Devices) in each of the two steps. The relative efficacy of the compounds was calculated as a percentage of the maximal effect induced by serotonin alone (defined as 100%). To determine EC₅₀/IC₅₀ values, concentration-response curves were fitted using a four-parameter logistic equation (IDB S Bio-book™). K_{b} values were calculated from IC₅₀ values, according to Cheng & Prusoff

### 3. Human 5-HT_{2B} Functional Assay

Functional activity on the 5-HT_{2B} receptor was determined by testing the effect of the compounds I on calcium mobilisation in CHO-FlpIn cells, stably transfected with human 5-HT_{2B} receptor. Cells were seeded into sterile black 384-well plates with clear bottom at 30,000 cells/well in a volume of 25 μl and grown overnight at 37° C., in 5% CO₂ in tissue culture medium (“CHO-S-SFM II” by Invitrogen), containing 1% dialysed FCS and 50 μg/ml gentamicin (Invitrogen). On the next morning, medium was replaced by a serum free version of the same tissue culture medium for a further incubation for 4 hours at 37° C. and in 5% CO₂. Cells were then loaded with a fluorescent cal-cium-sensitive dye in the presence of 0.07% probenecid for an hour at 37° C., according to the manufacturer's protocol (Ca5-Assay Kit, Molecular Devices), followed by an additional 60 min incubation at room temperature. Serial compound dilutions (final concentrations of 10⁻¹⁰ to 10⁻⁵M, prepared in HBSS+50 mM HEPES) were first added to the cells alone (“first addition” to assess agonism on the 5-HT_{2B} receptor), then after 8 min, serotonin was added to the same wells at a final concentration of 10⁻⁸ M (“second addition” to see potential antagonistic effect) and the maximal calcium response was determined using a FLIPR® Tetra instrument (Molecular Devices) in each of the two steps. The relative efficacy of the compounds was calculated as a percentage of the maximal effect induced by serotonin alone (defined as 100%). To determine EC₅₀/IC₅₀ values, concentration-response curves were fitted using a four-parameter logistic equation (IDBS Biobook™). K_{b} values were calculated from IC₅₀ values, according to Cheng & Prusoff.

### 4. Metabolic Stability

Samples of the tested compounds (0.5 μM) were preincubated together with rat liver microsomes (0.25 mg of microsomal protein/mL) in 0.05 M potassium phosphate buffer of pH 7.4 in microtiter plates at 37° C. for 5 minutes. The reaction was started by adding NADPH (1.0 mM). After 0, 5, 10, 15, 20 and 30 minutes, an aliquot was removed, the reaction was cooled and stopped by adding twice the amount of quench solution consisting of acetonitrile/methanol 1:1, and containing 0.2 M carbutamide as internal standard. The samples were frozen until analyzed. The remaining concentration of undegraded test substance was determined by liquid chromatography-tandem mass spectrometry (LC-MS/MS). The half-life (t_{1/2}) was determined from the gradient of the ratio of the signal of (test substance/internal standard)/unit time plot, allowing the calculation of the half-life of the test substance, assuming first order kinetics, from the decrease in the concentration of the compound with time. The microsomal clearance (mClint) was calculated as follows: mClint=((ln(2)/t 1/2)/Microsomal Protein Concentration (mg/ml))*1000, leading to the unit of uL/min/mg. The scaled clearance (mClin_scaled) was calculated as mCLint_scaled=m CLint*(Microsomal Yield (mg/kg BW))/1000000*60, leading to the units L/h/kg. The Microsomal Yield is defined by the specifics of the used microsomes. Calculations were modified from refer-ences: Di, The Society for Biomolecular Screening, 2003, 453-462; Obach, D M D, 1999 vol 27. N 11, 1350-1359.

### Unbound Fraction in Microsomes (Fu Mic)

A suspension of 0.25 mg/ml microsomal protein spiked with 0.5 μM of test compound was pipetted on one side of a HTDialysis device (HTDialysis LLC,37 Ledgewood Drive, Gales Fery Conn. 06335) separated by a membrane with a MWcut off 12-14 K. 50 mM K-Phosphate buffer (pH 7.4) was added on the other side of the well. After incubation at 37° C. for 4 h while shaking at 150 rpm, aliquots of both sides were taken, quenched with MeOH/ACN 1:1 and 0.2 μM of internal standard and frozen until analy-sis by LCMSMS.

### Calculation of Unbound Intrinsic Clearance

Cl int unbound=Cl int/fu mic

The compounds were used in form of their respective acid addition salts.

| | | | Cl int, mic |
|---|---|---|---|
| Ex. | EC50 | | unb.² (r) |
| No. | 5-HT_{2C}¹ | % efficacy | [l/h/kg] |
| 1 | +++ | +++ | + |
| 2 | ++ | +++ | + |
| 3 | +++ | +++ | + |
| 4 | ++ | +++ | + |
| 5 | +++ | +++ | + |
| 6 | + | +++ | + |
| 7 | + | +++ | + |
| 8 | + | +++ | +++ |
| 9 | + | +++ | ++ |
| 10 | + | +++ | ++ |
| 11 | ++ | +++ | + |
| 12 | ++ | +++ | + |
| 13 | +++ | +++ | + |
| 14 | ++ | +++ | + |
| 15 | ++ | +++ | + |
| 16 | + | +++ | +++ |
| 17 | ++ | +++ | +++ |
| 18 | ++ | +++ | +++ |
| 19 | + | +++ | +++ |
| 20 | +++ | +++ | +++ |
| 21 | +++ | +++ | +++ |
| 22 | ++ | +++ | +++ |
| 23 | +++ | +++ | +++ |
| 24 | ++ | +++ | +++ |
| 25 | +++ | +++ | ++ |
| 26 | + | +++ | +++ |
| 27 | ++ | +++ | ++ |
| 28 | ++ | +++ | +++ |
| 29 | ++ | +++ | +++ |
| 30 | +++ | +++ | +++ |
| 31 | ++ | +++ | +++ |
| 32 | ++ | +++ | +++ |
| 33 | +++ | +++ | +++ |
| 34 | ++ | +++ | + |
| 35 | +++ | +++ | ++ |
| 36 | + | +++ | ++ |
| 37 | ++ | +++ | ++ |
| 38 | + | +++ | + |
| 39 | + | +++ | ++ |
| 40 | + | +++ | ++ |
| 41 | +++ | ++ | +++ |
| 42 | +++ | +++ | +++ |
| 43 | + | +++ | +++ |

¹Potency (EC50 5-HT_{2C}) in functional assay
²unbound intrinsic clearance (human)
Potency (EC50):
+ from 15 nM to <30 nM
++ from 5 nM to <15 nM
+++ <5 nM
% Efficacy:
+ from 30 to 50%
++ from >50 to 70%
+++ >70%
Unbound intrinsic clearance:
+ from 80 to 250 l/h/kg
++ from 30 to <80 l/h/kg
+++ <30 l/h/kg

We claim:

1. A compound of formula I 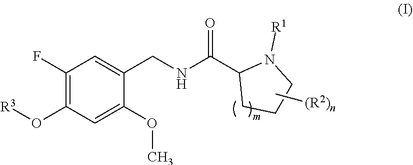
wherein
R¹ is hydrogen or methyl;
R² is fluoro or methyl;
R³ is selected from the group consisting of C₃-C₇-cycloalkyl which carries 1, 2, 3, 4, 5 or 6 substituents selected from the group consisting of fluoro and fluorinated C₁-C₄-alkyl; fluorinated C₁-C₈-alkyl; C₃-C₇-cycloalkyl-C₁-C₄-alkyl, where the cycloalkyl moiety carries 1, 2, 3, 4, 5 or 6 substituents selected from the group consisting of fluoro and fluorinated C₁-C₄-alkyl; phenyl-C₁-C₄-alkyl, where the phenyl ring carries 1, 2, 3, 4 or 5 substituents selected from the group consisting of fluoro and fluorinated C₁-C₄-alkyl, and may additionally carry one or more substituents selected from the group consisting of Cl, methyl and methoxy; and hetaryl-C₁-C₄-alkyl, where hetaryl is a 5- or 6-membered monocyclic heteroaromatic ring containing 1, 2 or 3 heteroatoms selected from the group consisting of N, O and S as ring members, where the heteroaryl ring carries 1, 2, 3, 4 or 5 substituents selected from the group consisting of fluoro and fluorinated C₁-C₄-alkyl, and may additionally carry one or more substituents selected from the group consisting of Cl, methyl and methoxy;
m is 0 or 1; and
n is 0 or 1;
or a stereoisomer or a pharmaceutically acceptable salt thereof, or the compound of the general formula I, wherein at least one of the hydrogen atoms has been replaced by deuterium.

2. The compound as claimed in claim 1, where R¹ is hydrogen.

3. The compound as claimed in any of the preceding claims, where R³ is C₄-C₆-cycloalkyl which carries 1 or 2 substituents selected from the group consisting of fluoro and fluorinated methyl.

4. The compound as claimed in any of claim 1 or 2, where R³ is fluorinated C₂-C₆-alkyl, where the carbon atom of the alkyl group which is bound to O does not carry any fluorine atom.

5. The compound as claimed in any of claim 1 or 2, where R³ is C₃-C₇-cycloalkyl-methyl, where the cycloalkyl moiety carries 1, 2, 3, 4, 5 or 6 substituents selected from the group consisting of fluoro and fluorinated methyl.

6. The compound as claimed in any of claim 1 or 2, where R³ is phenyl-C₁-C₂-alkyl, where the phenyl ring carries 1, 2, 3 or 4 substituents selected from the group consisting of fluoro and fluorinated methyl, and may additionally carry one Cl substituent.

7. The compound as claimed in any of claim 1 or 2, where R³ is hetaryl-C₁-C₂-alkyl, where hetaryl is a 5- or 6-membered monocyclic heteroaromatic ring containing 1 heteroatom selected from the group consisting of N and O as ring member, where the heteroaryl ring carries 1 or 2 substituents selected from the group consisting of fluoro and fluorinated methyl.

8. The compound as claimed in any of the preceding claims, where m is 1.

9. The compound as claimed in any of the preceding claims, where n is 0.

10. The compound as claimed in any of the preceding claims, where at least one of the hydrogen atoms of the moiety 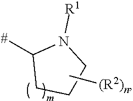
where # is the attachment point to C(O), has been replaced by a deuterium atom.

11. The compound as claimed in any of claims 1 to 9, of formula I.1 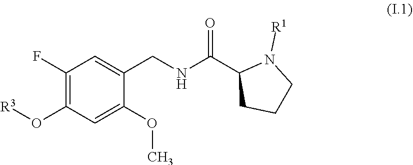
where R¹ and R³ are as defined in any of claims 1 to 7.

12. A compound selected from the group consisting of
(2S)—N-[[5-Fluoro-4-[(3-fluorophenyl)methoxy]-2-methoxy-phenyl]methyl]-pyrrolidine-2-carboxamide;
(2S)—N-[[5-Fluoro-2-methoxy-4-[(2,3,5-trifluorophenyl)methoxy]phenyl]methyl]pyrrolidine-2-carboxamide;
(2S)—N-[[5-Fluoro-4-[(4-fluorophenyl)methoxy]-2-methoxy-phenyl]methyl]-pyrrolidine-2-carboxamide;
(2S)—N-[[4-[(2,3-Difluorophenyl)methoxy]-5-fluoro-2-methoxy-phenyl]-methyl]-pyrrolidine-2-carboxamide;
(2S)—N-[[4-[(3,5-Difluorophenyl)methoxy]-5-fluoro-2-methoxy-phenyl]methyl]pyrrolidine-2-carboxamide;
(2S)—N-[[5-Fluoro-4-[(2-fluorophenyl)methoxy]-2-methoxy-phenyl]methyl]-pyrrolidine-2-carboxamide;
(2S)—N-[[4-[(2-Chloro-3,6-difluoro-phenyl)methoxy]-5-fluoro-2-methoxy-phenyl]methyl]pyrrolidine-2-carboxamide;
(2S)—N-[[5-fluoro-4-[(2-fluoro-4-pyridyl)methoxy]-2-methoxy-phenyl]methyl]pyrrolidine-2-carboxamide;
(2S)—N-[[5-Fluoro-4-[(5-fluoro-3-pyridyl)methoxy]-2-methoxy-phenyl]methyl]-pyrrolidine-2-carboxamide;
(2S)—N-[[5-fluoro-2-methoxy-4-[[6-(trifluoromethyl)-3-pyridyl]methoxy]phenyl]methyl]pyrrolidine-2-carboxamide;
(2S)—N-[[5-Fluoro-4-[2-(4-fluorophenyl)ethoxy]-2-methoxy-phenyl]methyl]-pyrrolidine-2-carboxamide;
(2S)—N-[[5-Fluoro-4-[2-(3-fluorophenyl)ethoxy]-2-methoxy-phenyl]methyl]-pyrrolidine-2-carboxamide;
(2S)—N-[[5-Fluoro-4-[2-(2-fluorophenyl)ethoxy]-2-methoxy-phenyl]methyl]-pyrrolidine-2-carboxamide;
(2S)—N-[[5-Fluoro-2-methoxy-4-[2-[4-(trifluoromethyl)phenyl]ethoxy]phenyl]methyl]pyrrolidine-2-carboxamide;
(2S)—N-[[5-fluoro-2-methoxy-4-[[5-(trifluoromethyl)-2-furyl]methoxy]phenyl]methyl]pyrrolidine-2-carboxamide;
(S)—N-(5-Fluoro-4-((cis-4-fluorocyclohexyl)oxy)-2-methoxybenzyl)pyrrolidine-2-carboxamide; fumaric
(S)—N-(5-Fluoro-4-((trans-4-fluorocyclohexyl)oxy)-2-methoxybenzyl)pyrrolidine-2-carboxamide;
(2S)—N-[[4-(4,4-Difluorocyclohexoxy)-5-fluoro-2-methoxy-phenyl]methyl]-pyrrolidine-2-carboxamide;
(2S)—N-[[5-Fluoro-2-methoxy-4-(3,3,3-trifluoropropoxy)phenyl]methyl]pyrrolidine-2-carboxamide;
(2S)—N-[[5-Fluoro-2-methoxy-4-(2,2,3,3-tetrafluoropropoxy)phenyl]methyl]-pyrrolidine-2-carboxamide;
(2S)—N-[[5-Fluoro-4-(2,2,3,4,4,4-hexafluorobutoxy)-2-methoxy-phenyl]methyl]pyrrolidine-2-carboxamide;
(2S)—N-[[5-Fluoro-2-methoxy-4-(2,2,3,3-tetrafluoro-1-methyl-propoxy)phenyl]methyl]pyrrolidine-2-carboxamide;
(2S)—N-[[5-Fluoro-2-methoxy-4-(4,4,4-trifluorobutoxy)phenyl]methyl]pyrrolidine-2-carboxamide;
(2S)—N-[[5-Fluoro-2-methoxy-4-(3,3,4,4,4-pentafluorobutoxy)phenyl]methyl]-pyrrolidine-2-carboxamide;
(2S)—N-[[5-Fluoro-2-methoxy-4-(4,4,4-trifluoro-2-methyl-butoxy)phenyl]methyl]pyrrolidine-2-carboxamide;
(2S)—N-[[4-[(2,2-Difluorocyclopropyl)methoxy]-5-fluoro-2-methoxy-phenyl]-methyl]pyrrolidine-2-carboxamide;
(2S)—N-[[5-Fluoro-2-methoxy-4-[[1-(trifluoromethyl)cyclopropyl]methoxy]-phenyl]methyl]pyrrolidine-2-carboxamide;
(2S)—N-[[4-(3,3-Difluorocyclobutoxy)-5-fluoro-2-methoxy-phenyl]methyl]-pyrrolidine-2-carboxamide;
(2 S)—N-[[4-[(3,3-Difluorocyclobutyl)methoxy]-5-fluoro-2-methoxy-phenyl]methyl]pyrrolidine-2-carboxamide;
(2S)—N-[[5-Fluoro-2-methoxy-4-[(2,2,3,3-tetrafluorocyclobutyl)methoxy]phenyl]methyl]pyrrolidine-2-carboxamide;
(2S)—N-[[4-[(3,3-Difluorocyclopentyl)methoxy]-5-fluoro-2-methoxy-phenyl]methyl]pyrrolidine-2-carboxamide;
(2S)—N-[[4-[(4,4-Difluorocyclohexyl)methoxy]-5-fluoro-2-methoxy-phenyl]methyl]pyrrolidine-2-carboxamide;
(S)—N-(5-Fluoro-4-((trans-4-trifluoromethyl-cyclohexyl)oxy)-2-methoxybenzyl)pyrrolidine-2-carboxamide;
(S)—N-(5-Fluoro-4-((cis-4-trifluoromethyl-cyclohexyl)oxy)-2-methoxybenzyl)-pyrrolidine-2-carboxamide;
(2S)—N-[[5-Fluoro-2-methoxy-4-[3-(trifluoromethyl)cyclohexoxy]phenyl]methyl]pyrrolidine-2-carboxamide;
(2S)—N-[[5-Fluoro-2-methoxy-4-(2,2,3,3-tetrafluoropropoxy)phenyl]methyl]-1-methyl-pyrrolidine-2-carboxamide;
(2S)—N-[[4-(3,3-Difluorocyclobutoxy)-5-fluoro-2-methoxy-phenyl]methyl]-1-methyl-pyrrolidine-2-carboxamide;
(2S)—N-[[4-[(3,3-Difluorocyclopentyl)methoxy]-5-fluoro-2-methoxy-phenyl]methyl]-1-methyl-pyrrolidine-2-carboxamide;
(2S)—N-(5-Fluoro-2-methoxy-4-((trans-4-(trifluoromethyl)cyclohexyl)oxy)-benzyl)-1-methyl-pyrrolidine-2-carboxamide;
(2S)—N-[[4-(4,4-Difluorocyclohexoxy)-5-fluoro-2-methoxy-phenyl]methyl]-1-methyl-pyrrolidine-2-carboxamide;
(2S)—N-[[5-Fluoro-2-methoxy-4-[trans 4-(trifluoromethyl)cyclohexoxy]phenyl]methyl]azetidine-2-carboxamide;
2,3,3,4,4,5,5-Heptadeuterio-N-[[5-fluoro-2-methoxy-4-[4-(trifluoromethyl)-cyclohexoxy]phenyl]methyl]pyrrolidine-2-carboxamide;
(2S)—N-[[5-Fluoro-4-[4-(fluoromethyl)cyclohexoxy]-2-methoxy-phenyl]-methyl]pyrrolidine-2-carboxamide;
or a stereoisomer, a stereoisomeric mixture or a pharmaceutically acceptable salt thereof.

13. A pharmaceutical composition comprising a therapeutically effective amount of at least one compound as claimed in any of the preceding claims or a stereoisomer or a pharmaceutically acceptable salt thereof, in combination with at least one pharmaceutically acceptable carrier and/or auxiliary substance.

14. The compound as claimed in any of claims 1 to 12 or a stereoisomer or a pharmaceutically acceptable salt thereof for use as a medicament.

15. The compound as claimed in any of claims 1 to 12 or a stereoisomer or a pharmaceutically acceptable salt thereof for use in the treatment of disorders which respond to the modulation of the 5-HT_{2c} receptor.

16. The use of a compound as claimed in any of claims 1 to 12 or of a stereoisomer or a pharmaceutically acceptable salt thereof for the manufacture of a medicament for the treatment of disorders which respond to the modulation of the 5-HT_{2c} receptor.

17. A method for treating disorders which respond to the modulation of the 5-HT_{2c} receptor, which method comprises administering to a subject in need thereof at least one compound as defined in any of claims 1 to 12 or a stereoisomer or a pharmaceutically acceptable salt thereof.

18. The compound as claimed in claim 15 or the use as claimed in claim 16 or the method as claimed in claim 17, where the disorders are selected from the group consisting of damage of the central nervous system, disorders of the central nervous system, eating disorders, ocular hypertension, cardiovascular disorders, gastrointestinal disorders and diabetes.

19. The compound or the use or the method as claimed in claim 18, where the disorders are selected from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease, behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, psychosis in Parkinson's disease, neuropsychiatric symptoms in Parkinson's disease, Lewy Body dementia, neuropsychiatric symptoms in Lewy Body dementia, spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

20. The compound or the use or the method as claimed in claim 19, where the disorders are selected from schizophrenia, depression, bipolar disorders, obesity, substance use disorders, neuropsychiatric symptoms in Alzheimer's disease and neuropsychiatric symptoms in Parkinson's disease.
